(19) United States
(12) Patent Application Publication
(10) Pub. No.: US 2008/0139472 A1
(43) Pub. Date: Jun. 12, 2008

(54) UPREGULATING BDNF LEVELS TO MITIGATE MENTAL RETARDATION

(75) Inventors: JULIE LAUTERBORN, Huntington Beach, CA (US); GARY LYNCH, Irvine, CA (US); CHRISTINE GALL, Irvine, CA (US); CHRISTOPHER REX, Irvine, CA (US)

Correspondence Address: BEYER WEAVER LLP, P.O. BOX 70250, OAKLAND, CA, 94612-0250, US

(73) Assignee: THE REGENTS OF THE UNIVERSITY OF CALIFORNIA

(21) Appl. No.: 11/868,336

(22) Filed: Oct. 5, 2007

Related U.S. Application Data
(60) Provisional application No. 60/849,925, filed on Oct. 6, 2006.
(60) Provisional application No. 60/977,011, filed on Oct. 2, 2007.

(51) Int. Cl. A61K 38/17; A61K 31/536; A61K 31/5365; A61K 31/549; A61K 31/465; A61K 31/197; A61K 31/135; A61P 43/00; A61K 31/405; A61K 31/138; A61K 31/565; A61K 31/145; A61K 31/454; A61K 31/498

(52) U.S. Cl. 514/12; 514/230.2; 514/229.5; 514/223.2; 514/322; 514/249; 514/665; 514/343; 514/182; 514/567; 514/653; 514/415

(57) ABSTRACT

This invention provides methods of preserving, improving, or restoring cognitive function in mammal having one or more mutations in the FMR1 gene (e.g. at risk for or having fragile x syndrome), where the methods involve the brain derived neurotrophic factor (BDNF) level or activity in the brain of said mammal. In certain embodiments the methods involve administering one or more AMPA potentiators (e.g., ampakines) to the mammal in an amount sufficient to increase BDNF levels in the brain of the mammal.

### CROSS-REFERENCE TO RELATED APPLICATIONS

This application claims benefit of and priority to U.S. Ser. No. 60/977,011, filed on Oct. 2, 2007 and U.S. Ser. No. 60/849,925, filed on Oct. 6, 2006, which are both incorporated herein by reference in their entirety for all purposes.

### STATEMENT AS TO RIGHTS TO INVENTIONS MADE UNDER FEDERALLY SPONSORED RESEARCH AND DEVELOPMENT

This invention was made with Government support under Grant No. NS004526 from the National Institutes of Health. The Government of the United States of America has certain rights in this invention.

### FIELD OF THE INVENTION

This invention pertains to the field of mental retardation. In particular this invention pertains to the discovery that elevating Brain Derived Neurotrophic Factor (BDNF) expression or activity can mitigate cognitive dysfunction in Fragile X syndrome.

### BACKGROUND OF THE INVENTION

Fragile X syndrome is the most commonly inherited form of mental retardation. Although it is thought to be an X-linked recessive trait with variable expression and incomplete penetrance, 30% of all carrier women are also affected. The syndrome is called “fragile-X” because there exists a fragile site or gap at the end of the long arm of the X-chromosome in lymphocytes of affected patients when grown in a folate deficient medium.

Fragile X syndrome is a genetic disorder caused by mutation of the FMR1 gene on the X chromosome. Mutation at that site is found in 1 out of about every 1250 males and 1 out of about every 2500 females. Normally, the FMR1 gene contains between 6 and 55 repeats of the CGG codon (trinucleotide repeats). In people with the fragile X syndrome, the FMR1 allele often has over 230 repeats of this codon.

Expansion of the CGG repeating codon to such a degree results in a methylation of that portion of the DNA, effectively silencing the expression of the FMR1 protein. This methylation of the FMR1 locus in chromosome band Xq27.3 is believed to result in constriction and fragility of the X chromosome at that point, a phenomenon that gave the syndrome its name.

Mutation of the FMR1 gene leads to the transcriptional silencing of the fragile X-mental retardation protein, FMRP. In normal individuals, FMRP binds and facilitates the translation of a number of essential neuronal RNAs. In fragile X patients, however, these RNAs are not translated into proteins and depending on the individual results in a number of conditions including, but not limited to mild to severe mental retardation, fragile X-associated tremor ataxia syndrome (FXTAS), and the like.

### SUMMARY

In various embodiments this invention provides methods of preserving, improving, or restoring cognitive function in mammal having one or more mutations in the FMR1 gene (e.g., fragile X syndrome and/or other cognitive disorders with little or no neural degradation). The methods typically involve increasing the brain derived neurotrophic factor (BDNF) level or activity in the brain of the mammal. In certain embodiments the methods involve administering one or more AMPA potentiators (e.g., ampakines). In certain embodiments the ampakines include high-impact ampakines.

Accordingly, in certain embodiments, methods are provided for preserving, improving, or restoring cognitive function in mammal having cognitive impairment and/or a learning disability. The methods typically involve increasing the level or activity of brain derived neurotrophic factor (BDNF) in the brain of said mammal. In certain embodiments the mammal shows no substantial neural degeneration. In certain embodiments the mammal shows essentially no measurable neural degeneration. In certain embodiments the mammal has a condition selected from the group consisting of Down's syndrome, autism, Rett's syndrome, nonsyndromic X-linked mental retardation, and fragile X syndrome. In certain embodiments the mammal is a mammal having one or more mutations in the FMR1 gene (e.g., a trinucleotide repeat expansion, abnormal methylation, etc.) and/or a mammal diagnosed as having, or at risk for, fragile X syndrome. In certain embodiments the preserving improving, or restoring cognitive function comprises improving long term potentiation in the hippocampus of the mammal. In certain embodiments the mammal is not diagnosed and/or under treatment for depression and/or an affective disorder. In certain embodiments increasing the BDNF level or activity comprises administering one or more glutamate AMPA receptor modulators (ampakines) to the mammal in an amount sufficient to upregulate expression or activity of BDNF in the mammal. In certain embodiments the glutamate AMPA receptor modulators comprise a high-impact ampakine (e.g., CX516, CX717, CX691, etc.). In certain embodiments the glutamate AMPA receptor modulators are compounds having the structure IVa or IVb as shown herein in which: Q and Q′ are independently hydrogen, —CH₂—, —O—, —S—, alkyl, or substituted alkyl, R¹ is hydrogen, alkyl or together with Q may be a cycloalkyl ring; R² may be absent, or if present may be —CH₂—, —CO—, —CH₂CH₂—, —CH₂CO—, —CH₂O—, —CRR′—, or —CONR—; Y is hydrogen or —OR³, or serves to link the aromatic ring to A as a single bond, ═N— or —NR—; R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring; A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur; R is hydrogen, aryl, arylalkyl, substituted aryl, substituted arylalkyl, alkyl, substituted alkyl, cycloalkyl, substituted cycloalkyl, or heterocycloalkyl; R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur; and X and X′ are independently R, halo, —CO₂R, —CN, —NRR′, —NRCOR′, —NO₂, —N₃ or —OR.

In certain embodiments the glutamate AMPA receptor modulators are compounds having the structure IVa, where: Q and Q′ are independently hydrogen, —CH₂—, —O—, —S—, alkyl, or substituted alkyl; R¹ is hydrogen, alkyl or together with Q may be a cycloalkyl ring, R² may be absent, or if present may be —CH₂—, —CO—, —CH₂CH₂—, —CH₂CO—, —CH₂O—, or —CONR—; Y is hydrogen or —OR³, or serves to link the aromatic ring to A as a single bond, ═N— or —NR—, R³ is hydrogen, alkyl, substituted alkyl, or serves to link the attached oxygen to A by being a lower alkylene such as a methylene or ethylene, or substituted lower alkylene such as—CRR′— linking the aromatic ring to A to form a substituted or unsubstituted 6, 7 or 8-membered ring, or a bond linking the oxygen to A in order to form a 5- or 6-membered ring, A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur, R is hydrogen, aryl, arylalkyl, substituted aryl, substituted arylalkyl, alkyl, substituted alkyl, cycloalkyl, substituted cycloalkyl, or heterocycloalkyl, R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y and which may optionally contain one or two heteroatoms such as oxygen, nitrogen or sulfur, X and X′ are independently R, halo, —CO₂R, —CN, —NRR′, —NRCOR′, —NO₂, —N₃ or —OR.

In certain embodiments the glutamate AMPA receptor modulators are compounds having the structure IVb where: Q and Q′ are independently hydrogen, —CH₂—, —O—, —S—, alkyl, or substituted alkyl, R¹ is hydrogen, alkyl or together with Q may be a cycloalkyl ring, R² may be absent, or if present may be—CH₂—, —CO—, —CH₂CH₂—, —CH₂CO—, —CH₂O—, or —CONR—, Y is hydrogen or —OR³, or serves to link the aromatic ring to A as a single bond, ═N— or —NR—, R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring, A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur; R is hydrogen, aryl, arylalkyl, substituted aryl, substituted arylalkyl, alkyl, substituted alkyl, cycloalkyl, substituted cycloalkyl, or heterocycloalkyl, R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur, X and X′ are independently R, halo, —CO₂R, —CN, —NRR′, —NRCOR′, —NO₂, —N₃ or —OR.

In certain of these embodiments Q and Q′ are —CH₂— and R² is —CH₂—. In certain of these embodiments and/or R¹ is hydrogen. In certain of these embodiments Q and Q′ are —CH₂— and R² is —CH₂CH₂—. In certain of these embodiments Q′ is —CH₂—, R² is —CH₂— and Q is —O— or —S—. In certain of these embodiments Q is —O—. In certain of these embodiments Q and Q′ are alkyl and R² is absent. In certain of these embodiments Q and Q′ are alkyl, R² is absent and R¹ is hydrogen. In certain of these embodiments Y is —OR³ and A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur. In certain of these embodiments A is alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur. In certain of these embodiments A is alkyl, substituted alkyl, cycloalkyl, substituted cycloalkyl, cycloalkylalkyl, a heterocycle or a substituted heterocycle containing one heteroatom such as oxygen, nitrogen or sulfur. In certain of these embodiments A is —NRR′, R is hydrogen, aryl, arylalkyl, substituted aryl, substituted arylalkyl, alkyl, substituted alkyl, cycloalkyl, substituted cycloalkyl, or heterocycloalkyl, R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and linked to Y by R³ and which may optionally contain one additional heteroatom such as oxygen, nitrogen or sulfur and X and X′ are independently R, halo, —CO₂R, —CN, —NRR′, —NRCOR′, —NO₂, —N₃ or —OR. In certain of these embodiments A is —NRR′, R is alkyl, substituted alkyl, cycloalkyl, substituted cycloalkyl, or heterocycloalkyl, R′ is hydrogen, alkyl, substituted alkyl, cycloalkyl, substituted cycloalkyl or may join together with R to form a 4- to 8-membered ring, which may be substituted by X and linked to Y by R³ and which may optionally contain one additional heteroatom such as oxygen, nitrogen or sulfur and X and X′ are independently R, halo, —CO₂R, —CN, —NRR′, —NRCOR′, —NO₂, —N₃ or —OR. In certain of these embodiments A is —NRR′ and R′ is joined together with R to form a 4- to 8-membered ring, which may be substituted by X and linked to Y by R³ and which may optionally contain one additional heteroatom such as oxygen, nitrogen or sulfur and X and X′ are independently R, halo, —CO₂R, —CN, —NRR′, —NRCOR′, —NO₂, —N₃ or —OR. In certain of these embodiments A is —NRR′, and R′ is joined together with R to form a 5-membered ring, which may be substituted by X and linked to Y by R.sup.3 and which may optionally contain one additional heteroatom such as oxygen, nitrogen or sulfur and X and X′ are independently R, halo, —CO₂R, —CN, —NRR′, —NRCOR′, —NO₂, —N₃ or —OR. In certain of these embodiments A is —NRR′, and R′ is joined together with R to form a 5-membered ring, which may be substituted by X and linked to Y by R³ and which may optionally contain one additional heteroatom such as oxygen, nitrogen or sulfur and X and X′ are independently R, halo, —CO₂R, —CN, —NRR′, —NRCOR′, —NO₂, —N₃ or —OR. In certain of these embodiments A is —NRR′, and R′ is joined together with R to form a 5-membered ring, which is linked to Y by R³. In certain of these embodiments A is —NRR′, and R′ is joined together with R to form a 6-membered ring, which may be substituted by X and linked to Y by R³ and which may optionally contain one additional heteroatom such as oxygen, nitrogen or sulfur and X and X′ are independently R, halo, —CO₂R, —CN, —NRR′, —NRCOR′, —NO₂, —N₃ or —OR. In various embodiments Y is —OR³. In various embodiments R³ is hydrogen. In various embodiments Y is hydrogen. In various embodiments Y is ═N— or —NR—. In various embodiments Y is ═N—. In various embodiments A is —OR, alkyl, substituted alkyl, cycloalkyl, substituted cycloalkyl, cycloalkylalkyl, a heterocycle or a substituted heterocycle containing one or two heteroatoms such as oxygen, nitrogen or sulfur. In various embodiments A is —NRR′. In various embodiments A is —OR, alkyl, substituted alkyl, cycloalkyl, substituted cycloalkyl, cycloalkylalkyl, a heterocycle or a substituted heterocycle containing one or two heteroatoms such as oxygen, nitrogen or sulfur. In various embodiments A is —NRR′. In various embodiments Y is —OR³ and A is —NRR′. In various embodiments R¹ is hydrogen.

In certain embodiments the glutamate AMPA receptor modulator comprises a compound in FIGS. 1-8 and/or a compound in Table 1 and/or LiD37 or D1.

In certain embodiments the method comprises administering BDNF or a BDNF analogue to the mammal. In certain embodiments the method comprises transfecting a neural cell with a construct that expresses a BDNF. In certain embodiments the method comprises administering to the mammal one or more agents selected from the group consisting of an anti-depressant drug, an anti-anxiolytic drug, an anti-psychotic drug, an acetylcholinesterase inhibitor, a delta- or mu-opioid receptor agonist, epidermal growth factor (EGF), nerve growth factor (NGF) and/or a bicyclic or tricyclic antidepressant and/or a selective serotonin reuptake inhibitor (SSRI) and/or an antidepressant selected from the group consisting of fluoxetine, desipramine, 2-methyl-6-(phenylethynyl)-pyridine), and Venlafaxine and/or an anxiolytic agent (e.g., afobazole, Buspirone, lorazepam, diazepam, fluoxetine, eszopiclone, paroxetine, sertaline, citalopram, clomipramine, clonazepram, St. John's wort, etc.) and/or an anti-psychotic (e.g., quetiapine, Chlorpromazine, fluphenazine, perphenazine, prochlorperazine, thioridazine, trifluoperazine, mesoridazine, promazine, triflupromazine, levomepromazine, chlorprothixene, flupenthixol, thiothixene, zuclopenthixol, haloperidol, droperidol, pimozide, melperone, clozapine, olanzapine, risperidone, quetiapine, ziprasidone, amisulpride, paliperidone, cannabidiol, LY2140023, etc.) and/or a histone deacetylase inhibitor (e.g., sodium butyrate, sodium phenylbutyrate, sodium phenylacetate, pivaloyloxymethylbutyrate, pyroxamide, Depsipeptide, Oxamflatin, benzamide derivative MS-275, trichostatin A, suberoylanilide hydroxamic acid, trapoxin A, trapoxin B, Cyl-1, Cyl-2, HC-toxin, WF-3161, chlamydocin, apicidin, MS-275 (previously called MS-27-275), depudecin, etc.) and/or an acetylcholinesterase inhibitor (e.g. huperzine A, physostigmine, pyridostigmine, ambenonium, demarcarium, edrophonium, neostigmine, tacrine (tetrahydroaminoacridine), donepezil (a.k.a. E2020), rivastigmine, metrifonate, galantamine, phenothiazine, etc.) and/or a neuropeptide whose expression is regulated by cocaine or other amphetamine, and/or cystamine or nictotine, and/or a monocyclic or bicyclic loop mimetic of BDNF, and/or estrogen or adrenocorticotropin, and/or dopamine, norepinephrine, LDOPA, serotonin, or analogues thereof, and/or Semax. In certain embodiments the agent comprises a compound that increases the activity of BDNF through up-regulating the BDNF receptor. In certain embodiments the method comprises improving or restoring congnitive function where the improved or restored cognitive function is characterized by improved learning ability or memory, reduced autistic-like behavior, improved attention, and/or reduced hypersensitivity to external stimuli.

Also provided is the use of a compound that increases the level or activity of BDNF in a mammal in the manufacture of a medicament for preserving, improving, or restoring cognitive function in mammal having cognitive impairment and/or a learning disability. In various embodiments the compound comprises any of the compounds described herein. In certain embodiments the mammal has a condition selected from the group consisting of Down's syndrome, autism, Rett's syndrome, nonsyndromic X-linked mental retardation, and fragile X syndrome. In certain embodiments the mammal has one or mutations in the FMR1 gene. In certain embodiments the medicament is for treatment or prevention one or more symptoms of fragile X syndrome in a mammal diagnosed with one or more mutations in the FMR1 gene. In certain embodiments the mammal shows no substantial neural degeneration and/or essentially no measurable neural degeneration. In certain embodiments the mammal is a mammal diagnosed as having, or at risk for, fragile X syndrome. In various embodiments the treatment or prevention comprises improving long term potentiation in the hippocampus of the mammal. In certain embodiments the compound comprises a glutamate AMPA receptor modulator as described herein.

Also provided are kits for preserving, improving, or restoring cognitive function in mammal having cognitive impairment and/or a learning disability. In certain embodiments the kits typically comprise a container containing one or more agents that increase the expression or activity of BDNF in a mammal (e.g., agents described herein); and instructional materials teaching the use of the agents to mitigate or prevent cognitive disorder in a mammal having or at risk for fragile X syndrome. In certain embodiments

In various embodiments, the methods of this invention expressly exclude the provision of particular exercise and/or dietary regimen. In various embodiments the methods exclude subjects diagnosed with and/or under treatment for a psychiatric disorder (e.g., an affective disorder) and/or expressly exclude the provision of antidepressants and/or anti-psychotics and/or anxioleptics, and/or opiates, and/or cannabinoids, and the like. Alternatively, or in addition to the above, the invention can also expressly exclude one or more of the drugs CX516, CX717, S19892, Org24448, Org26576, and GSK729327, 404187, LY 392098, and/or LY503430. In certain embodiments the invention expressly excludes one or more of the compounds described in U.S. Patent Publication 2004/0259871 (PCT Publication No: WO 2003/045315) and/or shown in FIG. 8. In certain embodiments the methods expressly exclude the Glaxo compound GSK729327. In certain embodiments the methods expressly exclude one or more of the compounds described in PCT Publication Nos: WO2006/087169, WO2006/015827, WO2006/015828, WO2006/015829, WO2007/090840, WO2007/090841, and WO2007/107539, e.g., one or more of the compounds in Table 1.

### Definitions

The phrase “increase BDNF level” refers to any method/process that increases the level and/or activity of BDNF in the brain. This includes, but is not limited to, the application of exogenous BNDF and/or BDNF analogues, expression of BDNF by engineered cells, expression of BDNF by autologous, heterologous, and/or homologous stem cells and/or progenitor cells, and/or by the use of agents induce BDNF expression and/or activity by brain cells and/or that facilitate release and/or processing of proBDNF to the mature form of BDNF, and/or retard breakdown of mature BDNF, which would therefore increase BDNF levels within the synaptic cleft.

An AMPA receptor refers to an AMPA-type (alpha-amino-3-hydroxy-5-methyl-isox-azole-4-propionic acid-type) glutamate receptor. AMPA receptors are found in high concentrations in neocortex (see, e.g., Petralia and Wenthold (1992) J. Comp. Neurol., 318: 329-354), in each of the major synaptic zones of hippocampus (see, e.g., Baude et al. (1995) Neurosci., 69: 1031-1055), and in the striatal complex (see Bernard et al. (1997) J. Neurosci., 17: 819-833).

The term “ampakines” refers to compounds (e.g., a class of modified benzamide compounds) that facilitate AMPA receptor mediated monosynaptic responses (EPSCs) in the brains of living animals.

The term “AMPA potentiators” refers to compounds that facilitate/potentiate the activity of AMPA receptors (see, e.g., Quirk and Nisenbaum (2002) CNS Drug Rev 8: 255-282; O'Neill et al. (2004) Curr Drug Targets CNS Neurol Disord 3: 181-194, and the like).

The terms “mammal” or “mammalian” refer to the class mammalia including the orders carnivore (e.g., dogs and cats), rodentia (e.g., mice, guinea pigs, and rats), and primates (e.g., humans, chimpanzees, and monkeys). In certain embodiments mammals include (canines, equines, felines, porcines, bovines, humans, and non-human primates).

As used herein, “brain tissue” means individual or aggregates of cells from or in the brain.

The terms “α-amino-3-hydroxy-5-methyl-isoxazole-4-proprionic acid receptor” or “AMPA receptor” refers to the class of glutamatergic receptors which are present in cells, particularly neurons, usually at their surface membrane that recognize and bind to glutamate or AMPA. The binding of AMPA or glutamate to an AMPA receptor normally gives rise to a series of molecular events or reactions that result in a biological response. The biological response may be the activation or potentiation of a nervous impulse, changes in cellular secretion or metabolism, causing the cells to undergo differentiation or movement, or increasing the levels of nucleic acids coding for neurotrophic factors or neurotrophic factor receptors.

An “effective amount” or “amount effective to” or “therapeutically effective amount” means a dosage sufficient to produce a desired result. Generally, the desired result is an increase in BDNF expression, availability, and/or activity.

A “low impact ampakine” refers to an ampakine that has little or no effect on the half-width of the field excitatory postsynaptic potential (fEPSP) in electrophysiology studies, and does not substantially bind to the cyclothiazide site on the AMPA receptor based upon binding studies. Illustrative low impact ampakines include, but are not limited to CX516, CX717, and Org24448.

A “high impact ampakine” refers to an refers to an ampakine that substantially alters (increase) the half-width of the field excitatory postsynaptic potential (fEPSP) in electrophysiology studies, and/or substantially bind to the cyclothiazide site on the AMPA receptor based upon binding studies.

The term “alkyl” is generally used herein to refer to a fully saturated monovalent radical containing carbon and hydrogen, and which may be a straight chain, branched or cyclic. In certain instances, the term alkyl can refer to both substituted and unsubstituted alkyl groups. Examples of alkyl groups include methyl, ethyl, n-butyl, n-heptyl, isopropyl, 2-methylpropyl, cyclopropyl, cyclopropylmethyl, cyclobutyl, cyclopentyl, cyclopentylethyl and cyclohexyl.

The term “substituted alkyl” refers to alkyl as just described including one or more functional groups such as lower alkyl containing 1-6 carbon atoms, aryl, substituted aryl, acyl, halogen (i.e., alkyl halos, e.g., CF₃), hydroxy, alkoxy, alkoxyalkyl, amino, alkyl and dialkyl amino, acylamino, acyloxy, aryloxy, aryloxyalkyl, carboxyalkyl, carboxamido, thio, thioethers, both saturated and unsaturated cyclic hydrocarbons, heterocycles and the like.

The term “aryl” refers to a substituted or unsubstituted monovalent aromatic radical having a single ring (e.g., phenyl) or multiple condensed rings (e.g., naphthyl). Other examples include heterocyclic aromatic ring groups having one or more nitrogen, oxygen, or sulfur atoms in the ring, such as imidazolyl, furyl, pyrrolyl, pyridyl, thienyl and indolyl.

The term “substituted aryl” refers to an aryl as just described that contains one or more functional groups such as lower alkyl, acyl, aryl, halogen, alkylhalos (e.g., CF₃), hydroxy, alkoxy, alkoxyalkyl, amino, alkyl and dialkyl amino, acylamino, acyloxy, aryloxy, aryloxyalkyl, carboxyalkyl, carboxamido, thio, thioethers, both saturated and unsaturated cyclic hydrocarbons, heterocycles and the like.

“Heterocycle” or “heterocyclic” refers to a carbocylic ring wherein one or more carbon atoms have been replaced with one or more heteroatoms such as nitrogen, oxygen or sulfur. The term encompasses both single ring structures and fused ring structures. Examples of heterocycles include, but are not limited to, piperidine, pyrrolidine, morpholine, thiomorpholine, piperazine, tetrahydrofuran, tetrahydropyran, 2-pyrrolidinone, Δ-velerolactam, .delta.-velerolactone and 2-ketopiperazine.

The term “substituted heterocycle” refers to a heterocycle as just described that contains one or more functional groups such as lower alkyl, acyl, aryl, cyano, halogen, hydroxy, alkoxy, alkoxyalkyl, amino, alkyl and dialkyl amino, acylamino, acyloxy, aryloxy, aryloxyalkyl, carboxyalkyl, carboxamido, thio, thioethers, both saturated and unsaturated cyclic hydrocarbons, heterocycles and the like.

The term “compound” is used herein to refer to any specific chemical compound disclosed herein. Within its use in context, the term generally refers to a single compound, but in certain instances may also refer to stereoisomers and/or optical isomers (including racemic mixtures) of disclosed compounds.

The term “sulfamoyl” refers to the —SO₂NH₂.

The term “alkoxy” denotes the group □OR (.quadrature.OR), where R is lower alkyl, substituted lower alkyl, aryl, substituted aryl, aralkyl or substituted aralkyl as defined below.

The term “acyl” denotes groups —C(O)R, where R is alkyl, substituted alkyl, alkoxy, aryl, substituted aryl, amino and alkylthiol.

A “carbocyclic moiety” denotes a ring structure in which all ring vertices are carbon atoms. The term encompasses both single ring structures and fused ring structures. Examples of aromatic carbocyclic moieties are phenyl and naphthyl.

The term “amino” denotes the group NRR′, where R and R′ may independently be hydrogen, lower alkyl, substituted lower alkyl, aryl, substituted aryl as defined below or acyl.

The term “amido” denotes the group —C(O)NRR′, where R and R′ may independently be hydrogen, lower alkyl, substituted lower alkyl, aryl, substituted aryl as defined below or acyl.

The term “subject” means a mammal, particularly a human. The term specifically includes domestic and common laboratory mammals, such as non-human primates, felines, canines, equines, porcines, bovines, goats, sheep, rabbits, rats and mice.

“Alpha-amino-3-hydroxy-5-methyl-isoxazole-4-propionic acid”, or “AMPA”, or “glutamatergic” receptors are molecules or complexes of molecules present in cells, particularly neurons, usually at their surface membrane, that recognize and bind to glutamate or AMPA. The binding of AMPA or glutamate to an AMPA receptor normally gives rise to a series of molecular events or reactions that result in a biological response. The biological response may be the activation or potentiation of a nervous impulse, changes in cellular secretion or metabolism, or causing cells to undergo differentiation or movement.

The phrase “effective amount” means a dosage sufficient to produce a desired result.

### BRIEF DESCRIPTION OF THE DRAWINGS

FIG. 1 shows that hippocampal LTP is impaired in young adult Fmr1-KO mice. Plots showing slopes of the field EPSPs in hippocampal slices from wild-type and fragile X mutant mice following theta burst stimulation. A single train of five theta bursts was delivered 10 minutes following stable baseline of the field EPSP (fEPSP) to the apical branch of the Schaffer-commissural projections and fEPSP responses to single pulses were collected from field CA1b for the following 40 min. Group data (mean ±sem) are expressed as percent of mean fEPSP slopes recorded during the baseline (pre-theta burst) period. As shown, Fmr1-KO slices (closed circles) expressed somewhat comparable initial potentiation to WTs (open circles), but the effect decayed rapidly to baseline by 20-30 min.

FIG. 2 shows that BDNF corrects LTP deficits in the Fragile X hippocampus. Plot showing field EPSPs in hippocampal slices from fragile X mutant mice following theta burst stimulation in the presence of brain derived neurotrophic factor (BDNF; 2 nM). BDNF treatment began 30 min prior to theta burst stimulation (stimulation parameters were as described in FIG. 1). In the presence of BDNF the potentiation in fragile X mouse hippocampus did not decay rapidly toward baseline, as observed in untreated slices (see FIG. 1 for comparison). Thus, by 40-50 min post-theta burst stimulation, the slope of the fEPSP was still enhanced 44% above baseline similar to wild-type responses.

FIGS. 3A, 3B, 3C illustrate compounds in accordance with Formula I of U.S. Pat. No. 6,166,008.

FIGS. 4A, 4B, and 4C illustrate compounds in accordance with Formula II of U.S. Pat. No. 6,166,008.

FIGS. 5A and 5B illustrate additional AMPA upregulator compounds.

FIG. 6 illustrates compounds in accordance with Formula III of U.S. Pat. No. 6,166,008.

FIG. 7 shows the structure of compound CX516, 1-(Quinoxalin-6-ylcarbonyl)piperidine.

FIG. 8 illustrates compounds in accordance with the formulas of U.S. Patent Publication 2004/0259871.

FIGS. 9A-9D show that hippocampal LTP is impaired in young adult Fmr1-KO mice. FIG. 9A: Plot of input-output curves generated from field responses to single pulse stimulation (duration increased in 0.02 ms steps) for Fmr1-KO (closed triangles) and WT (open circles) mice. FIG. 9B: A single train of 10 theta bursts was delivered (arrow, time 0) to the apical branch of the Schaffer commissural projections and fEPSP responses to single pulses were collected from field CA1b for the after 40 min. Group data (mean_SEM) are expressed as the percentage of mean fEPSP slopes recorded during the baseline (pre-theta burst) period. There were no reliable differences between WT (open circles) and mutant (closed circles) slices. Inset, Overlaid representative fEPSP traces collected during baseline and for 35 min (arrows) post-TBS for WT and Fmr1-KO mice. Calibration: 0.5 mV, 10 ms. FIG. 9C: Same as FIG. 9B except that the theta train contained only five bursts. Fmr1-KO slices (closed circles) expressed comparable initial potentiation to WTs, but the effect decayed rapidly to baseline by 30 min. FIG. 9D: Representative traces of fEPSPs for time points denoted in FIG. 9C (a, baseline; b, 35 min post-TBS) for slices from WT and Fmr1-KO mice. Calibration: 0.5 mV, 10 ms.

FIGS. 10A-10C show that the fragile X mutation does not impair events associated with the induction of LTP. FIG. 10A: The multiple fEPSPs in the composite response to a theta burst were normalized (by amplitude) to the first fEPSP in the first burst response. The responses for groups of slices were then averaged. FIG. 10A shows the averaged responses to the first and second theta bursts recorded from WT (n=8) and Fmr1-KO (n=7) slices. Note that the second burst response in each case is larger than the first and does not return as quickly to baseline (arrow in top trace). The superimposed traces (right side) indicate that the mutation does not affect the waveform of the composite response or its transformation within the train. FIG. 10B: Facilitation of burst responses within a theta train was estimated by expressing the area of responses 2-5 as a fraction of the first burst response. As shown, mean facilitation for both WT and Fmr1-KO slices was ˜80%. FIG. 10C: The size of the NMDA receptor contribution to the burst responses was estimated using the selective antagonist APV. A pair of theta bursts was delivered to the slice in the presence and absence of the compound. In WT slices, the effect of APV on the first burst response was limited to a slight reduction in the half-width of the fourth fEPSP, but on the second response it reduced the size of fEPSPs 2 through 4 (mean of 6 slices). Similar results were obtained in Fmr1-KO slices (mean of 7 slices).

FIGS. 11A-11C show that TBS-induced p-cofilin immunoreactivity is normal in Fmr1-KOs. FIGS. 11A and 11B: Laser confocal photomicrographs show p-cofilin-immunoreactivity in proximal CA1 stratum radiatum of hippocampal slices from WT (FIG. 11A) and Fmr1-KO (FX; FIG. 11B) mice that received either baseline lowfrequency stimulation (Ifs) or five TBSs; slices were collected 7 min after stimulation. Scale bar, 1 μm. FIG. 11C: Bar graph shows the number of p-cofilin-immunoreactive (ir) puncta (mean_SEM) per 100 μm2 for fields receiving Ifs (open bars) or TBS (closed bars) in WT and Fmr1-KO slices. Two-way ANOVA demonstrated a significant effect of TBS (p=0.00096), but no effect of genotype on p-cofilin-ir puncta counts. Thus, numbers of p-cofilin-ir puncta were significantly greater in slices that received TBS than in those that received Ifs for both WTs (**p=0.0019, t test; Ifs, n=3 mice; TBS, n=3 mice) and Fmr1-KOs (*p=0.033, t test; Ifs, n=3 mice; TBS, n=3 mice).

FIGS. 12A-12C show that Fmr1-KO mice show normal activity-dependent actin polymerization in dendritic spines. Acute hippocampal slices prepared from Fmr1-KO or WT mice were processed for in situ Alexafluor 568-phalloidin labeling of filamentous actin after electrophysiological recording in hippocampal region CA1. LTP was induced by TBS; control slices received baseline, lowfrequency stimulation (Ifs). FIG. 12A: Photomicrographs of Fmr1-KO (left) and WT (right) hippocampal slices showing representative phalloidin labeling in the field of afferent stimulation in CA1 stratum radiatum after TBS or Ifs. Scale bar, 10_m. FIG. 12B: Plot summarizes group mean (±SEM) numbers of densely phalloidin-labeled spine-like puncta per sample field for control/lfs (white bars) and TBS (black bars) slices. As indicated, TBS induced similar increases in the numbers of densely labeled spines between genotypes (**p<0.01 vs respective control group, Tukey's HSD after ANOVA). FIG. 12C: High-magnification photomicrographs show examples of densely phalloidin-labeled dendritic spines in CA1 stratum radiatum from Fmr1-KO and WTslices receiving TBS. Scale bar, 1 μm.

FIG. 13A-13E show that BDNF corrects the LTP deficit in fragile X hippocampus. FIG. 13A: Five theta bursts were delivered to the Schaffer commissural projections in WT and Fmr1-KO slices that had been treated with BDNF (50 ng/ml) beginning 30 min before theta stimulation. Potentiation in the mutants did not decay rapidly toward baseline, as observed in untreated slices (FIG. 1C) and did not differ in magnitude from the effect obtained in BDNF-treated WTslices. FIG. 13B: Mean fEPSP slope (average of 30-40 min post-TBS) expressed as a percentage of the last 10 min of baseline from Fmr1-KO slices either untreated (ACSF alone) or treated with BDNF or heat-inactivated BDNF. BDNF enhanced TBS-induced increases in the fEPSP slope compared with measures from the ACSF group (*p=0.009), whereas heat-inactivated BDNF had no effect. FIG. 13C: Group input-output data from Fmr1-KO slices treated with BDNF and heat-inactivated BDNF showed no effect of BDNF on fEPSP amplitude. FIG. 13D: Averaged responses to the first and fourth theta bursts recorded from Fmr1-KO slices infused with BDNF or with ACSF only. As shown, the response waveforms were comparable between the two groups. FIG. 13E: The effect of BDNF on burst response facilitation within a theta train in slices from fragile X mutant mice was estimated by expressing the area of responses 2-5 as a fraction of the first burst response. The mean degree of facilitation was similar in Fmr1-KO slices treated with BDNF and those bathed in ACSF alone.

FIGS. 14A and 14B show that hippocampal BDNF levels are normal in Fmr1-KO mice. FIG. 14A: Representative Western blot showing pro-BDNF (40-20 kDa) and mature BDNF (14 kDa) bands in hippocampal homogenates from WT and Fmr1-KO mice; band sizes (in kilodaltons) are indicated on the left. FIG. 14B: Bar graph showing quantification of hippocampal BDNF bands ranging in mass from 14 to 40 kDa for samples from WTs and Fmr1-KOs (n=6 per genotype). Plot shows BDNF band densities normalized to actin levels for the same sample. For each band, protein levels were equivalent between genotypes.

### DETAILED DESCRIPTION

This invention pertains to the surprising discovery that treatment of a mammal having or at risk for fragile X syndrome with BDNF results in a rescue effect. More generally, without being bound by a particular theory, it is believed that elevating levels (e.g., amount, expression, or activity) of BDNF or a BDNF analogue within the brain can be used as a method of treatment for cognitive impairment (e.g., mental retardation and/or learning disabilities), particularly cognitive impairment that is not associated with neural degeneration (e.g., neural cell death). More generally, without being bound to a particular theory, it is believed that administration of BDNF, a BDNF analogue, or a compound that increases BNDF expression and/or activity in the brain can be an effective treatment of mental retardation or cognitive impairment associated with diseases in which there is impaired synaptic plasticity without neurodegeneration as a causal factor.

It is shown in Example 1, that in a highly robust model of Fragile X syndrome, the Fmr1-knockout (KO) mouse, there is a deficit in hippocampal long term potentiation (LTP) that is fully restored to normal levels by application of Brain Derived Neurotrophic factor (BDNF). Importantly, the data indicates a rescue effect in a model of mental retardation that does not display neurodegeneration. These data indicate that elevation of BDNF level or activity within the brain, by any route feasible, can be used as a treatment for mental retardation, particularly cognitive deficit that is not associated with cell death.

As indicated above, it is believed that elevation of BDNF level or activity in the brain can be an effective treatment of mental retardation or cognitive impairment associated with diseases in which there is impaired synaptic plasticity without neurodegeneration as a causal factor. Examples of such diseases include, but are not limited to Fragile X, autism, Down's syndrome, and the like. In various embodiments the methods of the invention involve the use of BDNF, BDNF analogues, or methods of upregulating endogenous BDNF as a treatment for the impaired synaptic plasticity associated with the cognitive impairment.

### Increasing BDNF Levels.

BDNF can be either administered directly to the brain, or made in the brain via expression systems, genetically engineered cells, stem cells, or by any agent that can induce BDNF expression and/or activity by brain cells. Drugs that facilitate release of BNDF or proBNDF and/or processing of proBDNF to the mature form of BDNF, or retard breakdown of mature BDNF, which would therefore increase BDNF levels within the synaptic cleft, are also contemplated in certain embodiments of this invention.

Thus, in certain embodiments, methods are provided for preserving, and/or improving, and/or restoring cognitive function in mammal having one or more mutations in the FMR1 gene and/or cognitive impairment where there is no detectable and/or measurable and/or significant neural degeneration. The methods involve increasing the level or activity of brain derived neurotrophic factor (BDNF) level or activity in the brain of the mammal. In various embodiments the mammal is a human diagnosed as having, or at risk for, fragile X syndrome.

Various methods of increasing BDNF levels include, but are not limited to glutamate AMPA receptor modulators (e.g., ampakines) (see, e.g., U.S. Pat. No. 6,030,968 and US 2005/0228019 A1, which are incorporated herein by reference, e.g. for the compounds disclosed therein), physical exercise, dietary restriction, anti-depressant drugs (e.g. fluoxetine, desipramine, 2-methyl-6-(phenylethynyl)-pyridine), anti-anxiolytics (e.g. afobazole), histone deacetylase inhibitors (e.g. sodium butyrate), neuropeptides (e.g. cocaine- and amphetamine-regulated transcript), cystamine and related agents, nictotine, anti-psychotics (e.g. quetiapine, venlafaxine), and acetylcholinesterase inhibitors (e.g. huperzine A).

Also, compounds that mimic the effects of BDNF can also be effective. Such compounds include, but are not limited to peptides that are monocyclic and bicyclic loop mimetics of the neurotrophin. Furthermore, neurohormones (e.g. estrogen, adrenocorticotropin) and neurotransmitters and their precursors (e.g. dopamine, norepinephrine, LDOPA, serotonin) can up-regulate BDNF as well as compounds that mimic or increase levels of these neurochemicals (e.g. Semax is an analogue of the neurohormone adrenocorticotropin that increases BDNF levels). Finally, compounds that increase the activity of BDNF possibly through up-regulating its receptor (e.g. kinase inhibitors) are also viable therapeutics.

AMPA Potentiators/Ampakines.

In certain embodiments, the methods described herein involve administering one or more agents (e.g., ampakines and/or AMPA potentiators) that upregulate and/or potentiate AMPA receptors to a mammal characterized by substantial mutations in the FMR1 gene (e.g., having or at risk for Fragile X syndrome) and/or to a mammal having or at risk for cognitive impairment where there is little or no neural degeneration where the ampakines are provided at a level sufficient to increase BDNF level in the brain of the mammal.

A wide variety of AMPA receptor potentiators are useful in the present invention, including ampakines (see, e.g., PCT Publication No: WO 94/02475 (PCT/US93/06916), WO98/12185, U.S. Pat. Nos. 5,773,434, 6,030,968, 6,274,600, 6,166,008, and U.S. Patent Pub. 2005/0228019 A1 all of which are incorporated herein by reference in their entirety for all purposes); LY404187, LY 392098, LY503430, and derivatives thereof (produced by Eli Lilly, Inc.); CX546 and derivatives thereof; CX614 and derivatives thereof; St 8986-1 and derivatives thereof; benzoxazine AMPA receptor potentiators and derivatives thereof (see, e.g., U.S. Pat. Nos. 5,736,543, 5,962,447, 5,773,434 and 5,985,871 which are incorporated herein by reference in their entirety for all purposes); heteroatom substituted benzoyl AMPA receptor potentiators and derivatives thereof (see, e.g., U.S. Pat. Nos. 5,891,876, 5,747,492, and 5,852,008, which are herein incorporated by reference in their entirety for all purposes); benzoyl piperidines/pyrrolidines AMPA receptor potentiators and derivatives thereof as (see, e.g., U.S. Pat. No. 5,650,409, which is incorporated herein by reference in its entirety for all purposes); benzofurazan carboxamide AMPA receptor potentiators and derivatives thereof (see, e.g., U.S. Pat. Nos. 6,110,935, 6,313,1315 and 6,730,677, which are incorporated herein by reference for all purposes); 7-chloro-3-methyl-3-4-dihydro-2H-1,2,4 benzothiadiazine S,S, dioxide and derivatives thereof (see, e.g., Zivkovic et al. (1995), J. Pharmacol. Exp. Therap., 272: 300-309; Thompson et al. (1995) Proc. Natl. Acad. Sci., USA, 92: 7667-7671).

Illustrative ampakines include, but are not limited to CX546 (1-(1,4-benzodioxan-6-yl carbonyl)piperidine), CX516 (1-quinoxalan-6-yl-carbonyl)piperidine), CX614 (2H, 3H, 6aH pyrrolidino[2″, 1″-3′,2′]1,3-oxazino[6′,5′-5,4]benzo[e]1,4-dioxan-10-one), and CX929.

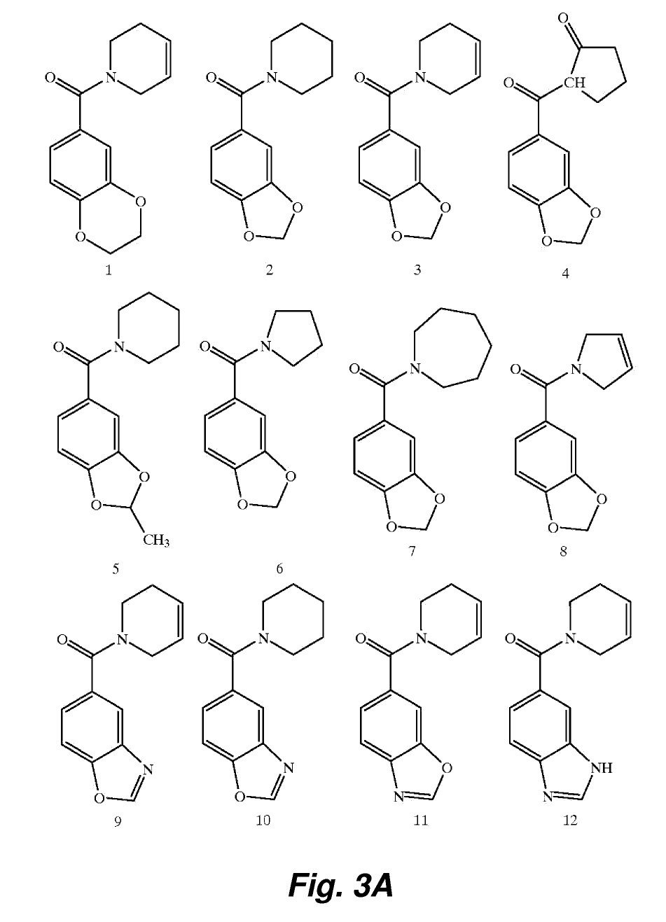

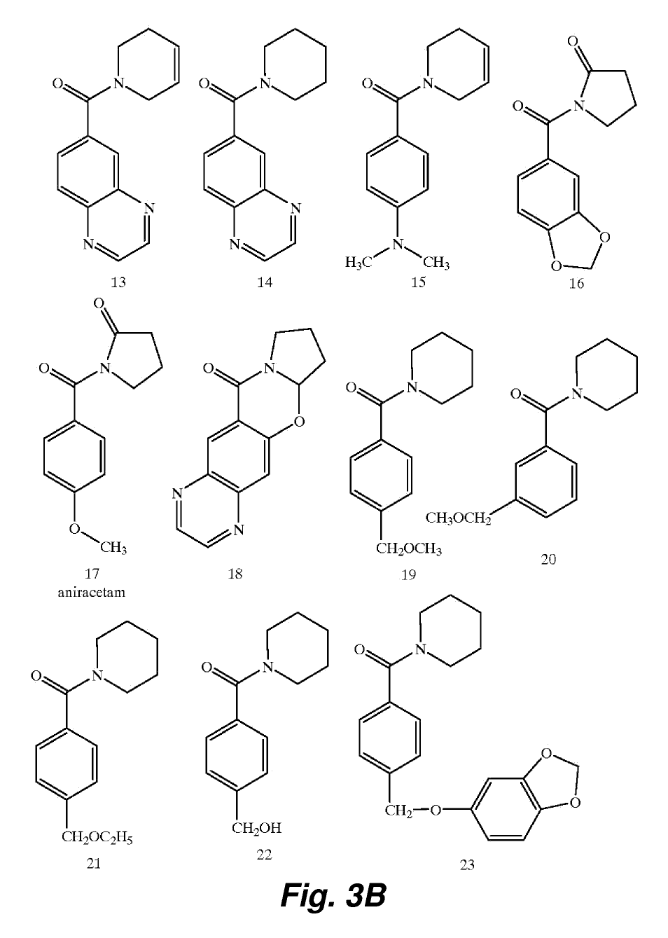

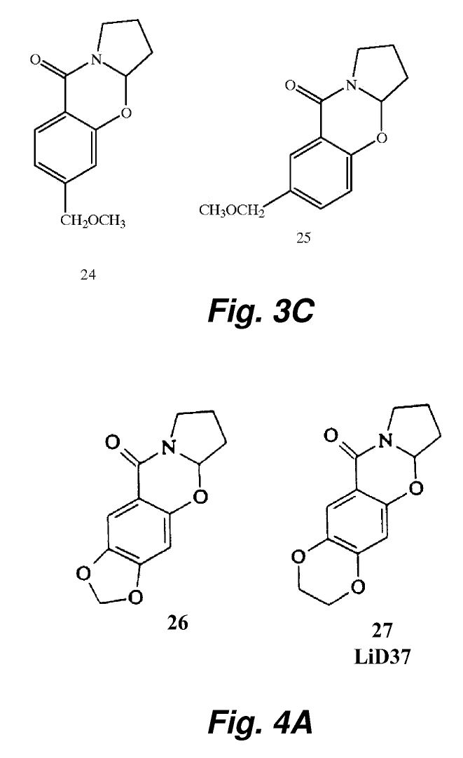

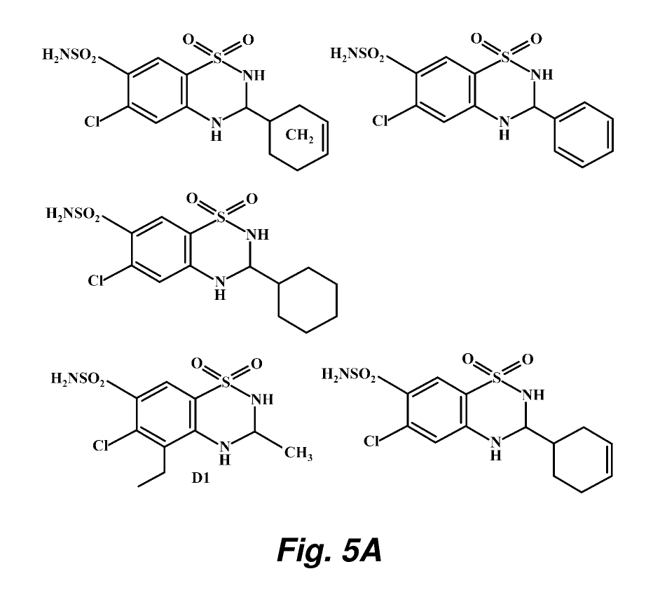

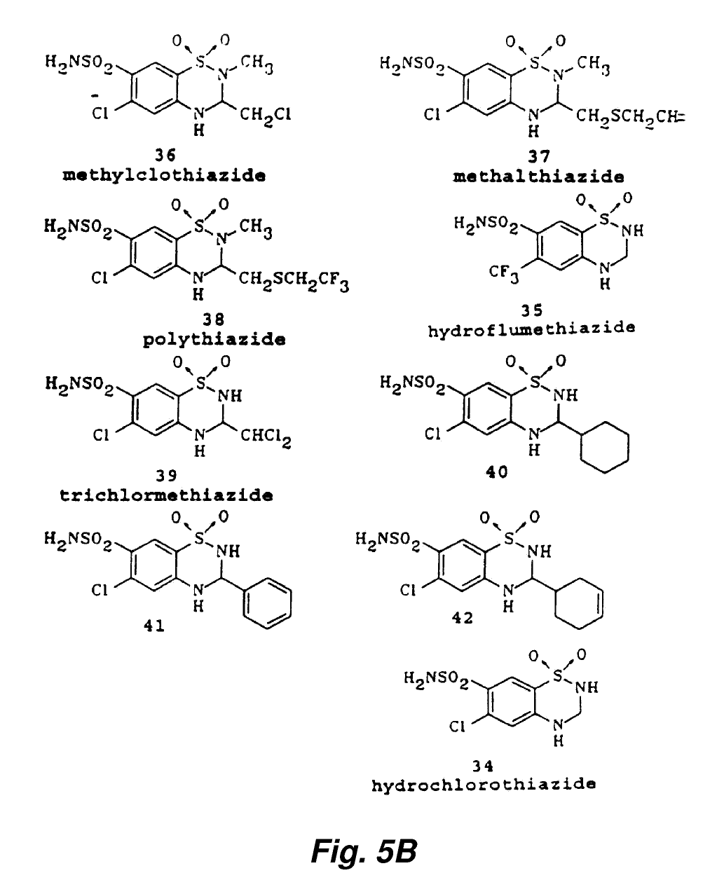

In certain embodiments particular compounds of interest include, but are not limited to: aniracetam, 7-chloro-3-methyl-3-4-dihydro-2H-1,2,4 benzothiadiazine S,S, dioxide, (see, e.g., Zivkovic et al. (1995) J. Pharmacol. Exp. Therap., 272: 300-309; Thompson et al. (1995) Proc. Natl. Acad. Sci., USA, 92:7667-7671) and those compounds shown in FIGS. 3-7.

In various embodiments the ampakine(s) include one or more high-impact ampakines.

In certain embodiments the methods of this invention utilize ampakines as described, for example, in U.S. Pat. No. 6,166,008. Such ampakines include, compounds according to formula I of U.S. Pat. No. 6,166,008:

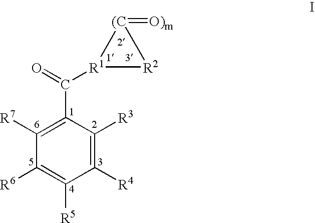

in which: R¹ is a member selected from the group consisting of N and CH; m is 0 or 1; R² is a member selected from the group consisting of (CR⁸₂)ₙ₋ₘ and Cₙ₋ₘR⁸₂₍ₙ₋ₘ₎₋₂, in which n is 4, 5, 6, or 7, the R⁸'s in any single compound being the same or different, each R⁸ being a member selected from the group consisting of H and C₁-C₆ alkyl, or one R⁸ being combined with either R³ or R⁷ to form a single bond linking the no. 3′ ring vertex to either the no. 2 or the no. 6 ring vertices or a single divalent linking moiety linking the no. 3′ ring vertex to either the no. 2 or the no. 6 ring vertices, the linking moiety being a member selected from the group consisting of CH₂, CH₂—CH₂, CH═CH, O, NH, N(C₁-C₆ alkyl), N═CH, N═C(C₁-C₆ alkyl), C(O), O—C(O), C(O)—O, CH(OH), NH—C(O), and N(C₁-C₆ alkyl)-C(O); R³, when not combined with any R⁸, is a member selected from the group consisting of H, C₁-C₆ alkyl, and C₁-C₆ alkoxy; R⁴ is either combined with R⁵ or is a member selected from the group consisting of H, OH, and C₁-C₆ alkoxy; R⁵ is either combined with R⁴ or is a member selected from the group consisting of H, OH, C₁-C₆ alkoxy, amino, mono(C₁-C₆ alkyl)amino, di(C₁-C₆ alkyl)amino, and CH₂ OR⁹, in which R⁹ is a member selected from the group consisting of H, C₁-C₆ alkyl, an aromatic carbocyclic moiety, an aromatic heterocyclic moiety, an aromatic carbocyclic alkyl moiety, an aromatic heterocyclic alkyl moiety, and any such moiety substituted with one or more members selected from the group consisting of C C₁-C₃ alkyl, C₁-C₃ alkoxy, hydroxy, halo, amino, alkylamino, dialkylamino, and methylenedioxy; R⁶ is either H or CH₂ OR⁹; R⁴ and R⁵, when combined, form a member selected from the group consisting of

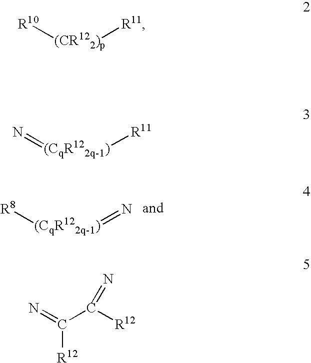

in which: R¹⁰ is a member selected from the group consisting of O, NH and N(C₁-C₆ alkyl); R¹¹ is a member selected from the group consisting of O, NH and N(C₁-C₆ alkyl); R¹² is a member selected from the group consisting of H and C₁-C₆ alkyl, and when two or more R¹²'s are present in a single compound, such R¹²'s are the same or different; p is 1, 2, or 3; and q is 1 or 2; and R⁷, when not combined with any R⁸, is a member selected from the group consisting of H, C₁-C₆ alkyl, and C₁-C₆ alkoxy. Compounds I through 25 in FIG. 3 are illustrative embodiments of compounds according to Formula I.

In certain embodiments the ampakines are ampakines according to Formula II of U.S. Pat. No. 6,166,008:

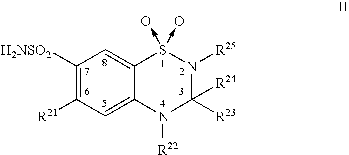

in which R²¹ is either H, halo or CF₃; R²² and R²³ either are both H or are combined to form a double bond bridging the 3 and 4 ring vertices; R²⁴ is either H, C₁-C₆ alkyl, C₅-C₇ cycloalkyl, C₅-C₇ cycloalkenyl, Ph (Ph denotes a phenyl group), CH₂Ph, CH₂SCH₂Ph, CH₂X, CHX₂, CH₂ SCH₂ CF₃, CH₂ SCH₂CH—CH₂, or

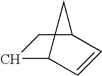

and R²⁵ is a member selected from the group consisting of H and C₁-C₆ alkyl.

Within the scope of Formula II, certain subclasses are preferred. One of these is the subclass in which R²¹ is C₁ or CF₃, with Cl preferred. Another is the subclass in which all X's are Cl. Still another is the subclass in which R²² and R²³ are both H. A preferred subclass of R²⁴ is that which includes CH₂Ph, CH₂SCH₂Ph, and

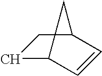

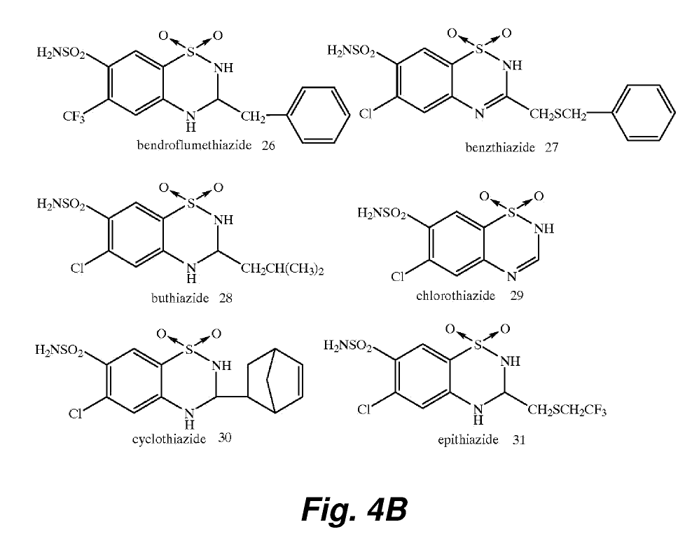

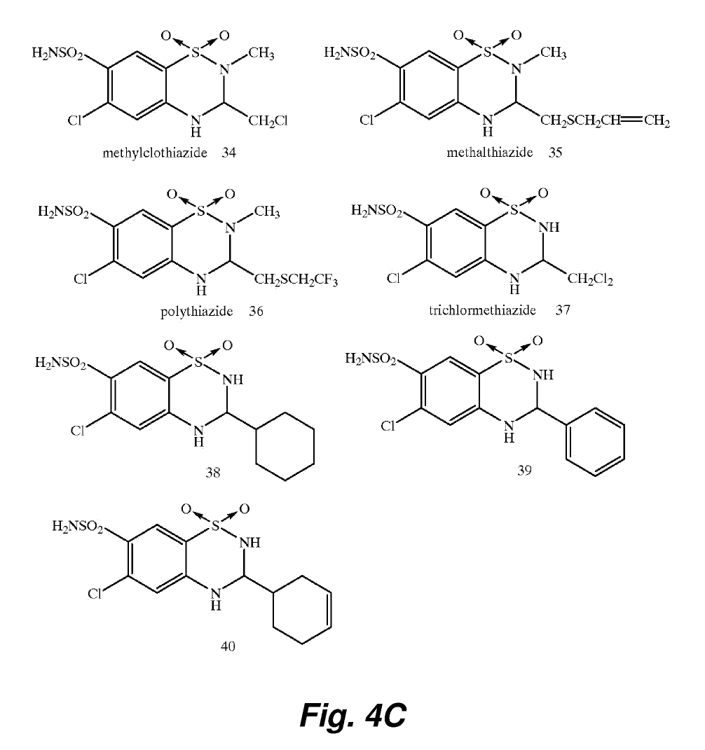

Compounds 26 through 40 in FIG. 4 are illustrative embodiments of compounds according to Formula II.

Certain preferred compounds within the scope of Formula II include those in which R²⁴ is either C₅-C₇ cycloalkyl, C₅-C₇ cycloalkenyl or Ph (“Ph” denotes a phenyl group). Other preferred compounds of this group are those in which R²¹ is halo, R²² is H, R²³ is H, and R²⁵ is H. Preferred substituents for R²⁴ include cyclohexyl, cyclohexenyl, and phenyl.

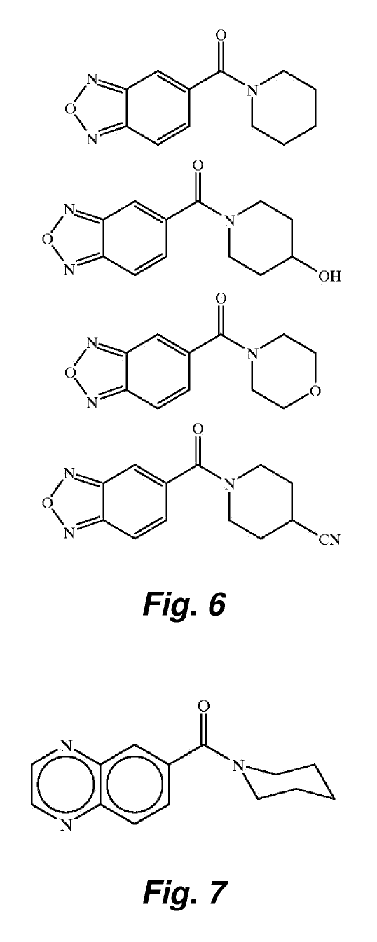

In another embodiment the ampakines are compounds according to Formula III of U.S. Pat. No. 6,166,008:

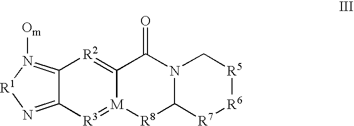

in which: R¹ is oxygen or sulfur; R² and R³ are independently selected from the group consisting of —N═, —CR═, and —CX═; M is ═N or ═CR⁴—, where R⁴ and R⁸ are independently R or together form a single linking moiety linking M to the ring vertex 2′, the linking moiety being selected from the group consisting of a single bond, —CR₂—, —CR═CR—, —C(O)—, —O—, —S(O)_{y}—, —NR—, and —N═; R⁵ and R⁷ are independently selected from the group consisting of—(C₂)ₙ—, —C(O)—, —CR═CR—, —CR═CX—, —C(RX)—, CX₂—, —S—, and —O—; and R₆ is selected from the group consisting of—(CR₂)ₘ—, —C(O)—, —CR═CR—, —C(RX)—, —CR₂—, —S—, and —O—; where X is—Br, —Cl, —F, —CN, —NO₂, —OR, —SR, —NR₂, —C(O)R—, —CO₂R, or —CONR₂; and R is hydrogen, C₁-C₆ branched or unbranched alkyl, which may be unsubstituted or substituted with one or more functionalities defined above as X, or aryl, which may be unsubstituted or substituted with one or more functionalities defined above as X; m and p are independently 0 or 1; n and y are independently 0, 1 or 2. Certain preferred embodiments include, but are not limited to the compounds in FIG. 6.

One particularly preferred compound is compound CX516, 1-(Quinoxalin-6-ylcarbonyl)piperidine, having the structure shown in FIG. 7.

The compounds described above are prepared by conventional methods known to those skilled in the art of synthetic organic chemistry. Numerous synthetic methods are described in U.S. Pat. No. 6,166,008 and the references cited therein.

In certain embodiments the compounds include, but are not limited to compounds described in U.S. Patent Publication 2004/0259871 (PCT Publication No: WO 2003/045315) which are incorporated herein by reference. Illustrative compounds have the structures IVa or IVb, below:

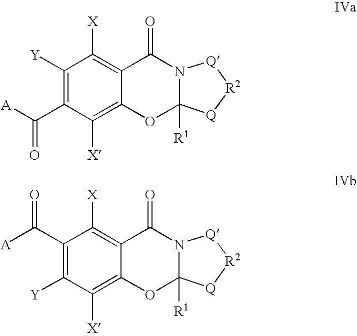

in which in which: Q and Q′ are independently hydrogen, —CH₂—, —O—, —S—, alkyl, or substituted alkyl; R¹ is hydrogen, alkyl or together with Q may be a cycloalkyl ring; R² may be absent, or if present may be—CH₂—, —CO—, —CH₂CH₂—, —CH₂CO—, —CH₂O—, —CRR′—, or —CONR—; Y is hydrogen or —OR³, or serves to link the aromatic ring to A as a single bond ═N— or —NR—; R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring; A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur; R is hydrogen, aryl, arylalkyl, substituted aryl, substituted arylalkyl, alkyl, substituted alkyl, cycloalkyl, substituted cycloalkyl, or heterocycloalkyl; R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur; X and X′ are independently R, halo, —CO₂R, —CN, —NRR′, —NRCOR′, —NO₂, —N₃ or —OR.

In certain embodiments, the compound is a compound according to structure IVa above where: Q and Q′ are independently hydrogen, —CH₂—, —O—, —S—, alkyl, or substituted alkyl, R¹ is hydrogen, alkyl or together with Q may be a cycloalkyl ring, R² may be absent, or if present may be—CH₂—, —CO—, —CH₂CH₂—, —CH₂CO—, —CH₂O—, or—CONR—, Y is hydrogen or —OR³, or serves to link the aromatic ring to A as a single bond, ═N— or —NR—, R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring, A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur, R is hydrogen, aryl, arylalkyl, substituted aryl, substituted arylalkyl, alkyl, substituted alkyl, cycloalkyl, substituted cycloalkyl, or heterocycloalkyl, R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y and which may optionally contain one or two heteroatoms such as oxygen, nitrogen or sulfur, X and X′ are independently R, halo, —CO₂R, —CN, —NRR′, —NRCOR′, —NO₂, —N₃ or —OR.

In certain embodiments, the compound is a compound according to structure IVb above where: Q and Q′ are independently hydrogen, —CH₂—, —O—, —S—, alkyl, or substituted alkyl, R¹ is hydrogen, alkyl or together with Q may be a cycloalkyl ring, R² may be absent, or if present may be—CH₂—, —CO—, —CH₂CH₂—, —CH₂CO—, —CH₂O—, or—CONR—, Y is hydrogen or —OR³, or serves to link the aromatic ring to A as a single bond, ═N— or —NR—, R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring, A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur; R is hydrogen, aryl, arylalkyl, substituted aryl, substituted arylalkyl, alkyl, substituted alkyl, cycloalkyl, substituted cycloalkyl, or heterocycloalkyl, R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur, X and X′ are independently R, halo, —CO₂R, —CN, —NRR′, —NRCOR′, —NO₂, —N₃ or —OR.

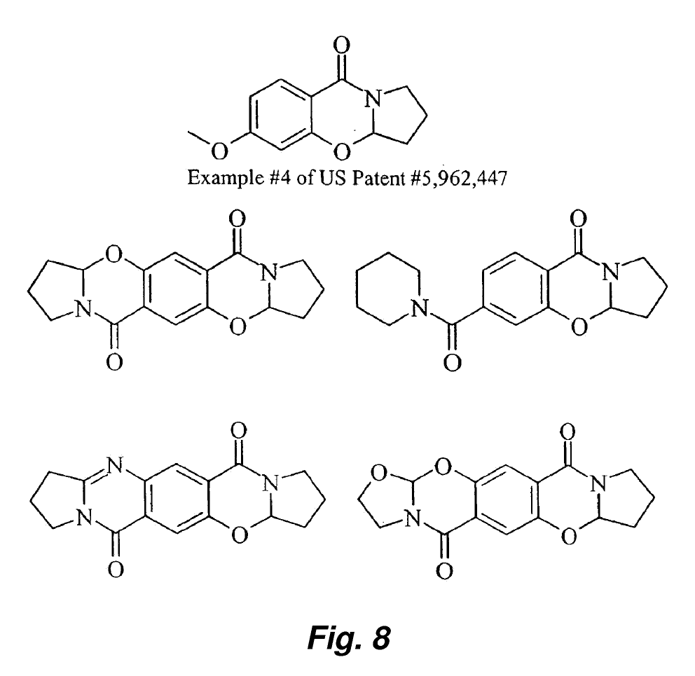

Illustrative compounds in accordance with these structures are shown in FIG. 8. Methods of making such compounds are described in U.S. Patent Publication 2004/0259871 (PCT Publication No: WO 2003/045315).

Other suitable AMPA receptor potentiators/ampakines include, but are not limited to the compounds disclosed in PCT Publication Nos: WO2006/087169, WO2006/015827, WO2006/015828, WO2006/015829, WO2007/090840, WO2007/090841, and WO2007/107539. Illustrative compounds disclosed in these applications are shown in

TABLE 1
Illustrative compounds disclosed in PCT Publication Nos:
WO2006/087169, WO2006/015827, WO2006/015828, WO2006/015829,
WO2007/090840, WO2007/090841, and WO2007/107539.

| PCT Publication | Compound Name |
|---|---|
| WO 2006/015827 | N-trans-(1-methyl-4-{3′-[(methylsulfonyl)amino]- |
| | 4-biphenylyl]-3-pyrrolidinyl)-2-propanesulfonamide |
| WO 2006/015827 | N-trans[4-(2′-fluoro-4-biphenylyl)-1-methyl-3- |
| | pyrrolidinyl]-2-propanesulfonamide |
| WO 2006/015827 | N-trans[-1-methyl-4-(4′-methyl-4-biphenylyl)-3- |
| | pyrrolidinyl]-2-propanesulfonamide |
| WO 2006/015827 | N-trans[-4-(4′-cyano-4-biphenylyl)-1-methyl-3- |
| | pyrrolidinyl]-2-propanesulfonamide |
| WO 2006/015827 | N-trans{-1-methyl-4-[3′-(methylsulfonyl)-4- |
| | biphenylyl]-3-pyrrolidinyl}-2-propanesulfonamide |
| WO 2006/015827 | N-trans{-1-methyl-4-[4-(3-thienyl)phenyl]-3- |
| | pyrrolidinyl1-2-propanesulfonamide |
| WO 2006/015827 | N-trans{-1-methyl-4-[4-(2-thienyl)phenyl]-3- |
| | pyrrolidinyl}-2-propanesulfonamide |
| WO 2006/015827 | N-trans{-1-methyl-413′-(trifluoromethyl)-4- |
| | biphenylyl]-3-pyrrolidinyl}-2-propanesulfonamide |
| WO 2006/015827 | N-trans{-1-methyl-4-[4-(5-pyrimidinyl)phenyl]-3- |
| | pyrrolidinyl}-2-propanesulfonamide |
| WO 2006/015827 | N-trans{-1-methyl-4-[4-(3-pyridyl)phenyl]-3- |
| | pyrrolidinyl}-2-propanesulfonamide |
| WO 2006/015827 | N-[4′-(trans-1-methyl-4-{[(1- |
| | methylethyl)sulfonyl]amino}-3-pyrrolidinyl)-3- |
| | biphenylyliacetamide |
| WO 2006/015827 | N-trans[-4-(3′-acetyl-4-biphenylyl)-1-methyl-3- |
| | pyrrolidinyl]-2-propanesulfonamide |
| WO 2006/015827 | N-{trans-4-4-(2-fluoro-3-pyridinyl)phenyl]-1- |
| | methyl-3-pyrrolidinyl}-2-propanesulfonamide |
| WO 2006/015827 | N-{trans-4-[4-(3-furanyl)phenyl]-1-methyl-3- |
| | pyrrolidinyl}-2-propanesulfonamide |
| WO 2006/015827 | N-trans{-4-[4-(1-benzothieN-3-yl)phenyl]-1- |
| | methyl-3-pyrrolidinyl}-2-propanesulfonamide |
| WO 2006/015827 | N-trans{-4-[4-(1,3-benzodioxol-5-yl)phenyl]- |
| | 1-methyl-3-pyrrolidinyl}-2-propanesulfonamide |
| WO 2006/015827 | N-trans{-1-methyl-4-[4′-(methyloxy)-4- |
| | biphenylyl]-3-pyrrolidinyl}-2-propanesulfonamide |
| WO 2006/015827 | Methyl4′-((trans)-1-methyl-4-{[(1- |
| | methylethyl)sulfonyl]amino}-3-pyrrolidinyl)-4- |
| | biphenylcarboxylate |
| WO 2006/015827 | N-(trans-1-methyl-4-{3′- |
| | [methyl(methylsulfonyl)amino]-4-biphenylyl}- |
| | 3-pyrrolidinyl)-2-propanesulfonamide |
| WO 2006/015827 | N-methyl-N-[4′-((trans)-1-methyl-4-{[(1- |
| | methylethyl)sulfonyl]amino}-3-pyrrolidinyl)- |
| | 3-biphenylyl]acetamide |
| WO 2006/015827 | N-trans[4-{3′-[(methylsulfonyl)amino]- |
| | 4-biphenylyl}-1-(phenylmethyl)-3-pyrrolidinyl]- |
| | 2-propanesulfonamide |
| WO 2006/015827 | Trans-N-(1-ethyl-4-{3′-[(methylsulfonyl)amino]- |
| | 4-biphenylyl}-3-pyrrolidinyl)-2-propanesulfonamide |
| WO 2006/015827 | N-[trans-4-(4′-cyano-4-biphenylyl)-1-ethyl-3- |
| | pyrrolidinyl]-2-propanesulfonamide |
| WO 2006/015827 | N-trans-(4-{3′-[(methylsulfonyl)amino]-4- |
| | biphenylyl1-3-pyrrolidinyl)-2-propanesulfonamide |
| WO 2006/015827 | N-trans-(1-(2-methylpropanoyl)-4-{3′- |
| | [(methylsulfonyl)amino]-4-biphenylyl}-3- |
| | pyrrolidinyl)-2-propanesulfonamide |
| WO 2006/015827 | Trans-N-(1-phenyl-4-{3′-[(methylsulfonyl)amino]- |
| | 4-biphenylyl}-3-pyrrolidinyl)-2-propanesulfonamide |
| WO 2006/015827 | Trans-N-[-4-(4′-cyano-4-biphenylyl)-1-phenyl-3- |
| | pyrrolidinyl]-2-propanesulfonamide |
| WO 2006/015827 | Trans-N-{-4-[4-(6-fluoro-3-pyridinyl)phenyl]- |
| | 1-phenyl-3-pyrrolidinyl}-2-propanesulfonamide |
| WO 2006/015827 | Trans-N-(-4-{3′-[methyl(methylsulfonyl)amino]- |
| | 4-biphenylyl}-1-phenyl-3-pyrrolidinyl)-2-propanesulfonamide |
| WO 2006/015827 | Trans-N-[-1-(2-methylpropyl)-4-{3′- |
| | [(methylsulfonyl)amino]-4-biphenylyl}- |
| | 3-pyrrolidinyl)-2-propanesulfonamide |
| WO 2006/015827 | Trans-N-[-4′-(-1-acetyl-4-{[(1- |
| | methylethyl)sulfonyl]amino}-3-pyrrolidinyl)- |
| | 3-biphenylyl]-N-(methylsulfonyl)acetamide |
| WO 2006/015827 | Trans-N-{-4-[4-(6-fluoro-3-pyridinyl)phenyl]- |
| | 1-methyl-3-pyrrolidinyl}-2-propanesulfonamide |
| WO 2006/015827 | Trans-N-[4-(-1-methyl-4-{[(1- |
| | methylethyl)sulfonyl]amino}-3- |
| | pyrrolidinyl)phenylibenzamide |
| WO 2006/015827 | Trans-N-(-1-(1-methylethyl)-4-{3′- |
| | [(methylsulfonyl)amino]-4-biphenylyl}-3- |
| | pyrrolidinyl)-2-propanesulfonamide |
| WO 2006/015827 | Trans-N-[-4-(4′-cyano-4-biphenylyl)-1-(1- |
| | methylethyl)-3-pyrrolidinyl]-2-propanesulfonamide |
| WO 2006/015828 | N-[5-(2-fluoro-3-pyridinyl)-2,3-dihydro-1H-indeN- |
| | 2-yl]-2-propanesulfonamide |
| WO 2006/015828 | N-[5-(6-fluoro-3-pyridinyl)-2,3-dihydro-1H-indeN- |
| | 2-yl]-2-propanesulfonamide |
| WO 2006/015828 | N-[5-(5-pyrimidinyl)-2,3-dihydro-1H-indeN-2- |
| | yl]-2-propanesulfonamide |
| WO 2006/015828 | N-[5-(3-thienyl)-2,3-dihydro-1H-indeN-2-yl]- |
| | 2-propanesulfonamide |
| WO 2006/015828 | N-[5-(3-pyridinyl)-2,3-dihydro-1H-indeN-2-yl]- |
| | 2-propanesulfonamide |
| WO 2006/015828 | N-[5-(2-thienyl)-2,3-dihydro-1H-indeN-2-yl]- |
| | 2-propanesulfonamide |
| WO 2006/015828 | N-[5-(4-methyl-3-pyridinyl)-2,3-dihydro-1H-indeN- |
| | 2-yl]-2-propanesulfonamide |
| WO 2006/015828 | N-[5-(2,6-dimethyl-3-pyridinyl)-2,3-dihydro-1H- |
| | indeN-2-yl]-2-propanesulfonamide |
| WO 2006/015828 | N-[5-(6-cyano-3-pyridinyl)-2,3-dihydro-1H-indeN- |
| | 2-yl]-2-propanesulfonamide |
| WO 2006/015828 | N-[5-(5-acetyl-3-pyridinyl)-2,3-dihydro-1H-indeN- |
| | 2-yl]-2-propanesulfonamide |
| WO 2006/015828 | N-[5-(5-cyano-3-pyridinyl)-2,3-dihydro-1H-indeN- |
| | 2-yl]-2-propanesulfonamide |
| WO 2006/015828 | N-[5-(5-fluoro-2-pyridinyl)-2,3-dihydro-1H-indeN- |
| | 2-yl]-2-propanesulfonamide |
| WO 2006/015828 | N-[5-(4-pyridinyl)-2,3-dihydro-1H-indeN-2-yl]- |
| | 2-propanesulfonamide |
| WO 2006/015828 | N-[5-(2-pyridinyl)-2,3-dihydro-1H-indeN-2-yl]- |
| | 2-propanesulfonamide |
| WO 2006/015828 | N-[5-(6-fluoro-2-pyridinyl)-2,3-dihydro-1H-indeN- |
| | 2-yl]-2-propanesulfonamide |
| WO 2006/015828 | N-[5-(2-methyl-4-pyridinyl)-2,3-dihydro-1H-indeN- |
| | 2-yl]-2-propanesulfonamide |
| WO 2006/015828 | N-[5-(6-methyl-3-pyridazinyl)-2,3-dihydro-1H-indeN- |
| | 2-yl]-2-propanesulfonamide |
| WO 2006/015828 | N-[5-(2-pyrimidinyl)-2,3-dihydro-1H-indeN-2-yl]- |
| | 2-propanesulfonamide |
| WO 2006/015828 | N-[5-(3-fluoro-4-pyridinyl)-2,3-dihydro-1H-indeN-2- |
| | yl]-2-propanesulfonamide |
| WO 2006/015828 | N-[5-(6-fluoro-2-methyl-3-pyridinyl)-2,3-dihydro-1 H- |
| | indeN-2-yl]-2-propanesulfonamide |
| WO 2006/015828 | N-[5-(1 H-imidazol-4-yl)-2,3-dihydro-1 H-indeN-2- |
| | yl]-2-propanesulfonamide |
| WO 2006/015828 | Ni5-(1,3,5-trimethyl-1H-pyrazol-4-yl)-2,3-dihydro-1H- |
| | indeN-2-yl]-2-propanesulfonamide |
| WO 2006/015828 | N-[5-(6-methyl-3-pyridinyl)-2,3-dihydro-1H-indeN-2- |
| | yl]-2-propanesulfonamide |
| WO 2006/015828 | N-[5-(3-methyl-2-pyridinyl)-2,3-dihydro-1H-indeN-2- |
| | yl]-2-propanesulfonamide |
| WO 2006/015828 | N-[5-(5-methyl-2-pyridinyl)-2,3-dihydro-1H-indeN-2- |
| | yl]-2-propanesulfonamide |
| WO 2006/015828 | N-[5-(6-chloro-3-pyridinyl)-2,3-dihydro-1H-indeN-2- |
| | yl]-2-propanesulfonamide |
| WO 2006/015828 | N-[5-[6-(methyloxy)-3-pyridinyl]-2,3-dihydro-1 H- |
| | indeN-2-yl}-2-propanesulfonamide |
| WO 2006/015828 | N-[5-(5-chloro-2-pyridinyl)-2,3-dihydro-1H-indeN-2- |
| | yl]-2-propanesulfonamide |
| WO 2006/015828 | N-[5-(2-chloro-3-pyridinyl)-2,3-dihydro-1H-indeN-2- |
| | yl]-2-propanesulfonamide |
| WO 2006/015828 | N-{(2S)-5[6-(trifluoromethyl)-3-pyridinyl]-2,3- |
| | dihydro-1H-indeN-2-yl}-2-propanesulfonamide |
| WO 2006/015828 | N-[(2S)-5-(5-chloro-2-pyridinyl)-2,3-dihydro-1 H-indeN- |
| | 2-yl]-2-propanesulfonamide |
| WO 2006/015828 | N-{(2S)-5[6-(trifluoromethyl)-2-pyridinyl]-2,3- |
| | dihydro-1H-indeN-2-yl}-2-propanesulfonamide |
| WO 2006/015828 | N-[(2S)-5-(5-methyl-3-pyridinyl)-2,3-dihydro-1H-indeN- |
| | 2-yl]-2-propanesulfonamide |
| WO 2006/015828 | N-[(2S)-5-(5-fluoro-3-pyridinyl)-2,3-dihydro-1H-IndeN- |
| | 2-yl]-2-propanesulfonamide |
| WO 2006/015828 | N-[(2S)-5-(2-fluoro-6-methyl-3-pyridinyl)-2,3-dihydro- |
| | 1H-indeN-2-yl]-2-propanesulfonamide |
| WO 2006/015828 | N-[(2S)-5-(2,6-difluoro-3-pyridinyl)-2,3-dihydro-1H- |
| | indeN-2-yl]-2-propanesulfonamide |
| WO 2006/015829 | N-(5-{4-[(methylsulfonyl)amino]phenyl}-2,3- |
| | dihydro-1H-indeN-2-yl)-2-propanesulfonamide |
| WO 2006/015829 | N-[3-(2-{[(1-methylethyl)sulfonyl]amino}- |
| | 2,3-dihydro-1H-indeN-5-yl)phenyl]acetamide |
| WO 2006/015829 | N-[5-(3-acetylphenyl)-2,3-dihydro-1H-indeN-2-yl]-2- |
| | propanesulfonamide |
| WO 2006/015829 | N-(5-{3-[methyl(methylsulfonyl)amino] phenyl}- |
| | 2,3-dihydro-1H-indeN-2-yl)-2-propanesulfonamide |
| WO 2006/015829 | N-[5-(3-{[(ethylamino)carbonyl]amino}phenyl)- |
| | 2,3-dihydro-1H-indeN-2-yl]-2-propanesulfonamide |
| WO 2006/015829 | N-(5-{3-[(ethylsulfonyl)amino]phenyl}- |
| | 2,3-dihydro-1H-indeN-2-yl)-2-propanesulfonamide |
| WO 2006/015829 | 2-methyl-N-[3-(2-{[(1- |
| | methylethyl)sulfonyl]amino}-2,3-dihydro-1H- |
| | indeN-5-yl)phenyl]propanamide |
| WO 2006/015829 | N-{5-[3-(2-oxopropyl)phenyl]-2,3-dihydro-1H-indeN- |
| | 2-yl}-2-propanesulfonamide |
| WO 2006/015829 | N-[5-(3-cyanophenyl)-2,3-dihydro-1H-indeN-2-yl]-2- |
| | propanesulfonamide |
| WO 2006/015829 | N-{5[3-(aminomethyl)phenyl]-2,3-dihydro-1H- |
| | indeN-2-yl}-2-propanesulfonamide |
| WO 2006/015829 | N-[5-(3-{[(methylsulfonyl)amino]methyl}phenyl)- |
| | 2,3-dihydro-1H-indeN-2-yl]-2-propanesulfonamide |
| WO 2006/015829 | N-{[3-(2-{[(1-methylethyl)sulfonyl]amino}- |
| | 2,3-dihydro-1H-indeN-5-yl)phenyl]methyl}acetamide |
| WO 2006/015829 | N-(5-{3-[(2-oxo-1-pyrrolidinyOmethyl]phenyl}- |
| | 2,3-dihydro-1H-indeN-2-yl)-2-propanesulfonamide |
| WO 2006/015829 | N-{5-[3-(1,1-dioxido-2-isothiazolidinyl)phenyl]- |
| | 2,3-dihydro-1H-indeN-2-yl}-2-propanesulfonamide |
| WO 2006/015829 | N-(5-{3-[(methylsulfonyl)methyl]phenyl}- |
| | 2,3-dihydro-1H-indeN-2-yl)-2-propanesulfonamide |
| WO 2006/015829 | 3-(2-{[dihydroxy(1-methylethyl)-λ⁴- |
| | sulfanyl]amino}-2,3-dihydro-1H-indeN-5-yl)- |
| | N,N-dimethylbenzenesulfonamide |
| WO 2006/015829 | 3-(2-{[dihydroxy(1-methylethyl)λ⁴sulfanyl]amino}- |
| | 2,3-dihydro-1H-indeN-5-yl)benzenesulfonamide |
| WO 2006/087169 | Trans-N-{-2-[4-(6-fluoro-3- |
| | pyridinyl)phenyl]cyclopropyl}-2- |
| | propanesulfonamide |
| WO 2006/087169 | Trans-N-{-2-[4-(6-methyl-3- |
| | pyridinyl)phenyl]cyclopropyl}-2- |
| | propanesulfonamide |
| WO 2006/087169 | Trans-N-{-2-[4-(5-fluoro-2- |
| | pyridinyl)phenyl]cyclopropyl}-2- |
| | propanesulfonamide |
| WO 2006/087169 | Trans-N-{-2-[4-(5-chloro-2- |
| | pyridinyl)phenyl]cyclopropyl}-2- |
| | propanesulfonamide |
| WO 2006/087169 | Trans-N-{-2-[4-(5-fluoro- |
| | phenyl)phenyl]cyclopropyl}-2- |
| | propanesulfonamide |
| WO 2006/087169 | Trans-N-{-2-[4-(4-cyano- |
| | phenyl)phenyl]cyclopropyl}-2- |
| | propanesulfonamide |
| WO 2006/087169 | Trans-N-{(2-[4-(1,3-benzodioxol-5- |
| | yl)phenyl]cyclopropyl}-2-propanesulfonamide |
| WO 2006/087169 | Trans-N-{-2-[3-(thienyl)phenyl]cyclopropyl}- |
| | 2-propanesulfonamide racemic |
| WO 2006/087169 | Trans-N-{-2-[2-(thienyl)phenyl]cyclopropyl}- |
| | 2-propanesulfonamide racemic |
| WO 2006/087169 | Trans-N-{-2-[4-(5-fluoro-3- |
| | pyridinyl)phenyl]cyclopropyl}-2- |
| | propanesulfonamide |
| WO 2006/087169 | Trans-N-{-2-[4-(5-methyl-3- |
| | pyridinyl)phenyl]cyclopropyl}-2- |
| | propanesulfonamide |
| WO 2006/087169 | Trans-N-{2-[4-(5-fluoro-3- |
| | pyridinyl)phenyl]cyclopropyl}-2- |
| | propanesulfonamide |
| WO 2006/087169 | Trans-N-{2-[4-(5-fluoro-3- |
| | pyridinyl)phenyl]cyclopropyl}-2- |
| | propanesulfonamide |
| WO 2006/087169 | Trans-N-{2-[4-(6-fluoro-3- |
| | pyridinyl)phenyl]cyclopropyl}-2- |
| | propanesulfonamide |
| WO 2006/087169 | Trans-N-{2-[4-(6-fluoro-3- |
| | pyridinyl)phenyl]cyclopropyl}-2- |
| | propanesulfonamide |
| WO 2006/087169 | Trans-N-{2-[4-(5-fluoro-2- |
| | pyridinyl)phenyl]cyclopropyl}-2- |
| | propanesulfonamide |
| WO 2006/087169 | Trans-N-{2-[4-(5-fluoro-2- |
| | pyridinyl)phenyl]cyclopropyl}-2- |
| | propanesulfonamide |
| WO 2006/087169 | Trans-N-{2-[4-(5-chloro-2- |
| | pyridinyl)phenyl]cyclopropyl}-2- |
| | propanesulfonamide |
| WO 2006/087169 | Trans-N-{2-[4-(5-chloro-2- |
| | pyridinyl)phenyl]cyclopropyl}-2- |
| | propanesulfonamide |
| WO 2006/087169 | Trans-N-[2-(4′-fluoro-4-biphenylyl)cyclopropyl]- |
| | 2-propanesulfonamide |
| WO 2006/087169 | Trans-N-[2-(4′-fluoro-4-biphenyly)0cyclopropyl]- |
| | 2-propanesulfonamide |
| WO 2006/087169 | Trans-N-[2-(4′-cyano-4-biphenyly)0cyclopropyl]- |
| | 2-propanesulfonamide |
| WO 2006/087169 | Trans-N-{2-[4-(1,3-benzodioxol-5- |
| | ylOphenyl]cyclopropyl}-2-propanesulfonamide |
| WO 2006/087169 | Trans-N-{2-[4-(5-methyl-3- |
| | pyridinyl)phenyl]cyclopropyl}-2- |
| | propanesulfonamide |
| WO 2006/087169 | Trans-N-{2-[4-(5-methyl-3- |
| | pyridinyl)phenyl]cyclopropyl}-2- |
| | propanesulfonamide |
| WO 2006/087169 | Trans-N-{2-[4-(2,2-difluoro-1,3-benzodioxol-5- |
| | yl)phenyl]cyclopropyl}-2-propanesulfonamide |
| WO 2006/087169 | Trans-N-{2-[3′-(methyloxy)-4- |
| | biphenylyl]cyclopropyl}-2-propanesulfonamide |
| WO 2006/087169 | Trans-N-{2-[4-(2-pyridinyl)phenyl]cyclopropyl}- |
| | 2-propanesulfonamide |
| WO 2006/087169 | Trans-N-{2-[4-(2-pyridinyl)phenyl]cyclopropyl}- |
| | 2-propanesulfonamide |
| WO 2006/087169 | Trans-N-(2-{4-[6-(methyloxy)-3- |
| | pyridinyl]phenyl}cyclopropyl)-2- |
| | propanesulfonamide |
| WO 2006/087169 | Trans-N-(2-{4-[3-(methyloxy)-2- |
| | pyridinyl]phenyl}cyclopropyl)-2- |
| | propanesulfonamide |
| WO 2006/087169 | Trans-N-(2-{4-[3-(methyloxy)-2- |
| | pyridinyl]phenyl}cyclopropyl)-2 - |
| | propanesulfonamide |
| WO 2006/087169 | Trans-N-{2-[4-(2-methyl-1,3-benzothiazol-5- |
| | yl)phenyl]cyclopropyl}-2-propanesulfonamide |
| WO 2007/090840 | N-{cis-4-[4-(6-fluoro-3-pyridinyl)phenyl]tetrahydro- |
| | 3-furanyl}-2-propanesulfonamide |
| WO 2007/090840 | N-{cis-4-[4-(6-methyl-3-pyridinyl)phenyl]tetrahydro- |
| | 3-furanyl}-2-propanesulfonamide |
| WO 2007/090840 | N-{cis-4-[4-(5-fluoro-2-pyridinyl)phenyatetrahydro- |
| | 3-furanyl}-2-propanesulfonamide |
| WO 2007/090840 | N-{cis-4-[4-(5-fluoro-3-pyridinyl)phenyl]tetrahydro- |
| | 3-furanyl}-2-propanesulfonamide |
| WO 2007/090840 | N-{cis-4-[4-(5-chloro-2-pyridinyl)phenyl]tetrahydro- |
| | 3-furanyl}-2-propanesulfonamide |
| WO 2007/090840 | N-{cis-4-[4-(5-methyl-3-pyridinyl)phenyl]tetrahydro- |
| | 3-furanyl}-2-propanesulfonamide |
| WO 2007/090840 | N-[cis-4-(4′-fluoro-4-biphenylyptetrahydro-3- |
| | furanyl]-2-propanesulfonamide |
| WO 2007/090840 | N-[cis-4-(4′-cyano-4-biphenylyptetrahydro-3- |
| | furanyl]-2-propanesulfonamide |
| WO 2007/090840 | N-[cis-4-(3′-acetyl-4-biphenylyptetrahydro-3- |
| | furanyl]-2-propanesulfonamide |
| WO 2007/090840 | N-{cis-4-[4-(1,3-benzodioxol-5-yl)phenyl]tetrahydro- |
| | 3-furanyl}-2-propanesulfonamide |
| WO 2007/090840 | N-{cis-4-[4-(3-thienyl)phenyl]tetrahydro-3- |
| | furanyl}-2-propanesulfonamide |
| WO 2007/090840 | N-{cis-4-[4-(2-thienyl)phenyl]tetrahydro-3- |
| | furanyl}-2-propanesulfonamide |
| WO 2007/090841 | N-{cis-2-[4-(6-fluoro-3-pyridinyl)phenyntetrahydro- |
| | 3-furanyl}-2-propanesulfonamide |
| WO 2007/090841 | N-{cis-2-[4-(6-methyl-3-pyridinyl)phenyl]tetrahydro- |
| | 3-furanyl)-2-propanesulfonamide |
| WO 2007/090841 | N-{cis-2-[4-(5-fluoro-2-pyridinyl)phenyl]tetrahydro- |
| | 3-furanyl)-2-propanesulfonamide |
| WO 2007/090841 | N-{cis-2-[4-(5-fluoro-3-pyridinyl)phenyl]tetrahydro- |
| | 3-furanyl)-2-propanesulfonamide |
| WO 2007/090841 | N-{cis-2-[4-(5-methyl-3-pyridinyl)phenyl]tetrahydro- |
| | 3-furanyl)-2-propanesulfonamide |
| WO 2007/090841 | N-[cis-2-(4′-fluoro-4-biphenylyptetrahydro-3- |
| | furanyl]-2-propanesulfonamide |
| WO 2007/090841 | N-[cis-2-(4′-cyano-4-biphenylyl)tetrahydro-3- |
| | furanyl]-2-propanesulfonamide |
| WO 2007/090841 | N-[cis-2-(3′-acetyl-4-biphenylyptetrahydro-3- |
| | furanyl]-2-propanesulfonamide |
| WO 2007/090841 | N-{cis-2-[4-(2-thienyl)phenyl]tetrahydro-3- |
| | furanyl}-2-propanesulfonamide |
| WO 2007/107539 | N,N-dimethyl-4[3-(trifluoromethyl)-4,5,6,7-tetrahydro- |
| | 1H-indazol-1-yl]benzamide |
| WO 2007/107539 | 1-[-4-(1-pyrrolidinylcarbonyl)phenyl]-3- |
| | (trifluoromethyl)-4,5,6,7-tetrahydro-1H-indazole |
| WO 2007/107539 | N-methyl-N-(2-phenylethyl)-443-(trifluoromethyl)- |
| | 4,5,6,7-tetrahydro-1H-indazol-1-yl]benzamide |
| WO 2007/107539 | N-ethyl-N-methyl-4[3-(trifluoromethyl)-4,5,6,7- |
| | tetrahydro-1H-indazol-1-yl]benzamide |
| WO 2007/107539 | N-butyl-N-methyl-4[3-(trifluoromethyl)-4,5,6,7- |
| | tetrahydro-1H-indazol-1-yl]benzamide |
| WO 2007/107539 | N-methyl-N-(2-phenylethyl)-4-[3-(trifluoromethyl)- |
| | 4,5,6,7-tetrahydro-1H-indazol-1-yl]benzamide |
| WO 2007/107539 | N,N-dimethyl-4[3-(trifluoromethyl)-4,5,6,7- |
| | tetrahydro-1H-indazol-1-yl]benzenesulfonamide |
| WO 2007/107539 | 1-{4[3-(trifluoromethyl)-4,5,6,7-tetrahydro-1H- |
| | indazol-1-yl]phenyllethanone |
| WO 2007/107539 | 1-{4[3-(trifluoromethyl)-4,5,6,7-tetrahydro-1H- |
| | indazol-1-yl]phenyl}-1-propanone |
| WO 2007/107539 | 1-[4-(methylsulfonyl)phenyl]-3-(trifluoromethyl)- |
| | 4,5,6,7-tetrahydro-1H-indazole |
| WO 2007/107539 | 1-{4[3-(trifluoromethyl)-4,5,6,7-tetrahydro-1H- |
| | indazol-1-yl]phenyl1-2-propanone |
| WO 2007/107539 | N,N-dimethyl-2-{4[3-(trifluoromethyl)-4,5,6,7- |
| | tetrahydro-1H-indazol-1-yl]phenyl}acetamide |
| WO 2007/107539 | 1-{4-[2-oxo-2-(1-pyrrolidinypethyl]phenyl1-3- |
| | (trifluoromethyl)-4,5,6,7-tetrahydro-1H-indazole |
| WO 2007/107539 | N-ethyl-N-methyl-2-{4[3-(trifluoromethyl)-4,5,6,7- |
| | tetrahydro-1H-indazol-1-yl]phenyllacetamide |
| WO 2007/107539 | N-methyl-N-(phenylmethyl)-2-{443-(trifluoromethyl)- |
| | 4,5,6,7-tetrahydro-1H-indazol-1-yl]phenyllacetamide |
| WO 2007/107539 | N-butyl-N-methyl-2-{4[3-(trifluoromethyl)-4,5,6,7- |
| | tetrahydro-1H-indazol-1-yl]phenyllacetamide |
| WO 2007/107539 | N-methyl-N-(2-phenylethyl)-2-{443-(trifluoromethyl)- |
| | 4,5,6,7-tetrahydro-1H-indazol-1-yl]phenyl}acetamide |
| WO 2007/107539 | 1-{[4-(1-pyrrolidinylcarbonyl)phenyl]methyl1-3- |
| | (trifluoromethyl)-4,5,6,7-tetrahydro-1H-indazole |
| WO 2007/107539 | 1-{4-[1-methyl-2-oxo-2-(1- |
| | pyrrolidinypethyl]phenyl}-3-(trifluoromethyl)- |
| | 4,5,6,7-tetrahydro-1H-indazole |
| WO 2007/107539 | N,N-dimethyl-3-{4[3-(trifluoromethyl)-4,5,6,7- |
| | tetrahydro-1H-indazol-1-yl]phenyllpropanamide |
| WO 2007/107539 | 1-{4-[3-oxo-3-(1-pyrrolidinyl)propyl]phenyl}- |
| | 3-(trifluoromethyl)-4,5,6,7-tetrahydro-1H-indazole |
| WO 2007/107539 | 1-{4-[1-(1-pyrrolidinylcarbonyl)cyclopropyl]phenyl}-3- |
| | (trifluoromethyl)-4,5,6,7-tetrahydro-1H-indazole |
| WO 2007/107539 | 1-{4-[2-oxo-2-(1-piperidinypethyl]phenyl}- |
| | 3-(trifluoromethyl)-4,5,6,7-tetrahydro-1H-indazole |
| WO 2007/107539 | 1-{4-[2-(3,3-difluoro-1-pyrrolidinyl)-2- |
| | oxoethyl]phenyl1-3-(trifluoromethyl)-4,5,6,7- |
| | tetrahydro-1H-indazole |
| WO 2007/107539 | N-methyl-N-propyl-2-{4-[3-(trifluoromethyl)- |
| | 4,5,6,7-tetrahydro-1H-indazol-1-yl]phenyl}acetamide |
| WO 2007/107539 | N-cyclopentyl-2-{4[3-(trifluoromethyl)-4,5,6,7- |
| | tetrahydro-1H-indazol-1-yl]phenyl}acetamide |
| WO 2007/107539 | N-methyl-N-(2-thienylmethyl)-2-{4-[3- |
| | (trifluoromethyl)-4,5,6,7-tetrahydro-1H-indazol- |
| | 1-yl]phenyl}acetamide |
| WO 2007/107539 | {4-[3-(trifluoromethyl)-4,5,6,7-tetrahydro-1H- |
| | indazol-1-yl]phenyl}acetonitrile |
| WO 2007/107539 | {4-[3-(trifluoromethyl)-4,5,6,7-tetrahydro-1H- |
| | indazol-1-yl]phenyllmethanol |
| WO 2007/107539 | N-methyl-N-({4[3-(trifluoromethyl)-4,5,6,7- |
| | tetrahydro-1H-indazol-1-yl]phenyl}methypacetamide |
| WO 2007/107539 | 1-({4[3-(trifluoromethyl)-4,5,6,7-tetrahydro-1H- |
| | indazol-1-yl]phenyl}methyl)-2-pyrrolidinone |
| WO 2007/107539 | N-methyl-N-({4[3-(trifluoromethyl)-4,5,6,7- |
| | tetrahydro-1H-indazol-1-yl]phenyl}methyl)propanamide |
| WO 2007/107539 | N-ethyl-N-({4[3-(trifluoromethyl)-4,5,6,7- |
| | tetrahydro-1H-indazol-1-yl]phenyl}methyl)acetamide |
| WO 2007/107539 | 1-({4[3-(trifluoromethyl)-4,5,6,7-tetrahydro-1H- |
| | indazol-1-yl]phenyl}methyl)-2-piperidinone |
| WO 2007/107539 | 1-methyl-5-{4[3-(trifluoromethyl)-4,5,6,7- |
| | tetrahydro-1H-indazol-1-yl]phenyl}-2-pyrrolidinone |
| WO 2007/107539 | N-[3-(1H-imidazol-1-yl)propyl]-N-methyl-4-[3- |
| | (trifluoromethyl)-4,5,6,7-tetrahydro-1H-indazol-1- |
| | yl]benzamide |
| WO 2007/107539 | N-methyl-N-[2-(2-thienypethyl]-443-(trifluoromethyl)- |
| | 4,5,6,7-tetrahydro-1H-indazol-1-yl]benzamide |
| WO 2007/107539 | N-methyl-N-[2-(1H-1,2,4-triazol-1-yl)ethyl]-4-[3- |
| | (trifluoromethyl)-4,5,6,7-tetrahydro-1H-indazol-1- |
| | yl]benzamide |
| WO 2007/107539 | N-methyl-N-(1,3-thiazol-2-ylmethyl)-4-[3- |
| | (trifluoromethyl)-4,5,6,7-tetrahydro-1H-indazol-1- |
| | yl]benzamide |
| WO 2007/107539 | N-methyl-N-[2-(1-methyl-1H-pyrrol-2-yl)ethyl]-4- |
| | [3-(trifluoromethyl)-4,5,6,7-tetrahydro-1H-indazol- |
| | 1-yl]benzamide |
| WO 2007/107539 | N-methyl-N-(2-thienylmethyl)-4-[3-(trifluoromethyl)- |
| | 4,5,6,7-tetrahydro-1H-indazol-1-yl]benzamide |
| WO 2007/107539 | N-methyl-N-(3-pyridinylmethyl)-4-[3-(trifluoromethyl)- |
| | 4,5,6,7-tetrahydro-1H-indazol-1-yl]benzamide |
| WO 2007/107539 | N-(2-furanylmethyl)-N-methyl-4-[3-(trifluoromethyl)- |
| | 4,5,6,7-tetrahydro-1H-indazol-1-yl]benzamide |
| WO 2007/107539 | N-[(4-fluorophenyl)methyl]-N-methyl-4-[3- |
| | (trifluoromethyl)-4,5,6,7-tetrahydro-1H-indazol-1- |
| | yl]benzamide |
| WO 2007/107539 | 1-[4-(morpholinylcarbonyl)phenyl]-3- |
| | (trifluoromethyl)-4,5,6,7-tetrahydro-1H-indazole |
| WO 2007/107539 | N-({4[3-(trifluoromethyl)-4,5,6,7-tetrahydro-1H- |
| | indazol-1-yl]phenyl}methyl)methanesulfonamide |
| WO 2007/107539 | 1-{4-[1-fluoro-2-oxo-2-(1- |
| | pyrrolidinypethyl]phenyl}-3-(trifluoromethyl)- |
| | 4,5,6,7-tetrahydro-1H-indazole |
| WO 2007/107539 | 1-{4-[1,1-difluoro-2-oxo-2-(1-pyrrolidinypethyl]phenyl1- |
| | 3-(trifluoromethyl)-4,5,6,7-tetrahydro-1H-indazole |
| WO 2007/107539 | N-methyl-N-({4[3-(trifluoromethyl)-4,5,6,7-tetrahydro- |
| | 1H-indazol-1-yl]phenyl}methyl)methanesulfonamide |
| WO 2007/107539 | 1-(4-{[3-(trifluoromethyl)-4,5,6,7-tetrahydro-1H- |
| | indazol-1-yl]methyl}phenyl)-2-pyrrolidinone |
| WO 2007/107539 | N-methyl-N-({4[3-(trifluoromethyl)-4,5,6,7-tetrahydro- |
| | 1H-indazol-1-yl]phenyl}methyl)-1-pyrrolidinecarboxamide |
| WO 2007/107539 | 5-{4-[3-(trifluoromethyl)-4,5,6,7-tetrahydro-1H- |
| | indazol-1-yl]phenyl}-2-pyrrolidinone |
| WO 2007/107539 | N-(1-{4-[3-(trifluoromethyl)-4,5,6,7-tetrahydro-1H- |
| | indazol-1-yl]phenyl}ethypacetamide |
| WO 2007/107539 | N-methyl-N-(1-{4-[3-(trifluoromethyl)-4,5,6,7- |
| | tetrahydro-1H-indazol-1-yl]phenyl}ethyl)acetamide |
| WO 2007/107539 | 1-[4-(1-acetyl-2-pyrrolidinyl)phenyl]-3- |
| | (trifluoromethyl)-4,5,6,7-tetrahydro-1H-indazole |
| WO 2007/107539 | 1-(2-{4[3-(trifluoromethyl)-4,5,6,7-tetrahydro-1H- |
| | indazol-1-yl]phenyl}ethyl)-2-pyrrolidinone |
| WO 2007/107539 | 1-{4-[(1,1-dioxido-2- |
| | isothiazolidinyl)methyl]phenyl}-3- |
| | (trifluoromethyl)-4,5,6,7-tetrahydro-1H-indazole |
| WO 2007/107539 | 2-methyl-N-({4[3-(trifluoromethyl)-4,5,6,7- |
| | tetrahydro-1H-indazol-1-yl]phenyl}methyl)propanamide |
| WO 2007/107539 | N-({4-[3-(trifluoromethyl)-4,5,6,7-tetrahydro- |
| | 1H-indazol-1-yl]phenyl}methyl)butanamide |
| WO 2007/107539 | N-({4-[3-(trifluoromethyl)-4,5,6,7-tetrahydro- |
| | 1H-indazol-1-yl]phenyl}methyl)-2-thiophenecarboxamide |
| WO 2007/107539 | N-({4[3-(trifluoromethyl)-4,5,6,7-tetrahydro-1H- |
| | indazol-1-yl]phenyl}methyl)propanamide |
| WO 2007/107539 | N-({4[3-(trifluoromethyl)-4,5,6,7-tetrahydro-1H- |
| | indazol-1-yl]phenyl}methypacetamide |
| WO 2007/107539 | N-methyl-2-phenyl-N-({443-(trifluoromethyl)-4,5,6,7- |
| | tetrahydro-1H-indazol-1-yl]phenyl}methyl)acetamide |
| WO 2007/107539 | N-(2-hydroxyethyl)-N-methyl-4-[3-(trifluoromethyl)- |
| | 4,5,6,7-tetrahydro-1H-indazol-1-yl]benzamide |
| WO 2007/107539 | N-methyl-N-[2-(methyloxy)ethyl]-4-[3- |
| | (trifluoromethyl)-4,5,6,7-tetrahydro-1H-indazol- |
| | 1-yl]benzamide |
| WO 2007/107539 | N-methyl-N-[2-(methylamino)ethyl]-4-[3- |
| | (trifluoromethyl)-4,5,6,7-tetrahydro-1H-indazol-1- |
| | yl]benzamide |
| WO 2007/107539 | 1-({4[3-(trifluoromethyl)-4,5,6,7-tetrahydro-1H- |
| | indazol-1-yl]phenyl}carbonyl)-3-pyrrolidinol |
| WO 2007/107539 | N-methyl-1-({4[3-(trifluoromethyl)-4,5,6,7- |
| | tetrahydro-1H-indazol-1-yl]phenyl}carbonyl)-3- |
| | pyrrolidinamine |
| WO 2007/107539 | 1-[4-(1-azetidinylcarbonyl)phenyl]-3- |
| | (trifluoromethyl)-4,5,6,7-tetrahydro-1H-indazole |
| WO 2007/107539 | 1-({4-[3-(trifluoromethyl)-4,5,6,7-tetrahydro- |
| | 1H-indazol-1-yl]phenyl}carbonyl)-3-azetidinol |
| WO 2007/107539 | (3,3-difluorocyclobutyl){4-[3-(trifluoromethyl)- |
| | 4,5,6,7-tetrahydro-1H-indazol-1-yl]phenyl}methanone |
| WO 2007/107539 | 1-[4-(1H-imidazol-1-yl)phenyl]-3-(trifluoromethyl)- |
| | 4,5,6,7-tetrahydro-1H-indazole |
| WO 2007/107539 | N-({4[3-(trifluoromethyl)-4,5,6,7-tetrahydro-1H- |
| | indazol-1-yl]phenyl}methyl)-2-propenamide |
| WO 2007/107539 | N-(1-methylethenyl)-N-({443-(trifluoromethyl)-4,5,6,7- |
| | tetrahydro-1H-indazol-1-yl]phenyl}methyl)-2-propenamide |
| WO 2007/107539 | N-methyl-N-({4[3-(trifluoromethyl)-4,5,6,7-tetrahydro- |
| | 1H-indazol-1-yl]phenyl}methyl)-2-propenamide |
| WO 2007/107539 | 1-{4-[(3-methyl-1,2,4-oxadiazol-5- |
| | yl)methyl]phenyl}-3-(trifluoromethyl)- |
| | 4,5,6,7-tetrahydro-1H-indazole |
| WO 2007/107539 | 1-{4-[(3-cyclopropyl-1,2,4-oxadiazol-5- |
| | yl)methyl]phenyl}-3-(trifluoromethyl)- |
| | 4,5,6,7-tetrahydro-1H-indazole |
| WO 2007/107539 | N-ethyl-4-[3-(trifluormethyl)-4,5,6,7-tetrahydro- |
| | 1H-indazol-1-yl]benzamide |
| WO 2007/107539 | N-methyl-N-(1-methylethyl)-4-[3-(trifluoromethyl)- |
| | 4,5,6,7-tetrahydro-1H-indazol-1-yl]benzamide |
| WO 2007/107539 | 1-[4-(1-piperidinylcarbonyl)phenyl]-3- |
| | (trifluormethyl)-4,5,6,7-tetrahydro-1H-indazole |
| WO 2007/107539 | N,N-diethyl-4-[3-(trifluormethyl)-4,5,6,7-tetrahydro- |
| | 1H-indazol-1-yl]benzamide |
| WO 2007/107539 | N-methyl-4-[3-trifluoromethyl)-4,5,6,7-tetrahydro- |
| | 1H-indzaol-1-yl]benzamide |
| WO 2007/107539 | 1-{4-[2-oxo-2-(2-phenyl-1- |
| | pyrrolidinypethyl]phenyl1-3-(trifluoromethyl)- |
| | 4,5,6,7-tetrahydro-1H-indazole |
| WO 2007/107539 | N-methyl-N-({4[3-(trifluoromethyl)-4,5,6,7- |
| | tetrahydro-1H-indazol-1-yl]phenyl}methyl)benzamide |
| WO 2007/107539 | 1-[4-(1,3-oxazol-5-yl)phenyl]-3-(trifluoromethyl)- |
| | 4,5,6,7-tetrahydro-1H-indazole |
| WO 2007/107539 | 1-[4-(propyloxy)phenyl]-3-(trifluoromethyl)- |
| | 4,5,6,7-tetrahydro-1H-indazole |
| WO 2007/107539 | 1-[4-(1-methyl-1H-imidazol-4-yl)phenyl]-3- |
| | (trifluoromethyl)-4,5,6,7-tetrahydro-1H-indazole |
| WO 2007/107539 | N-({4[3-(trifluoromethyl)-4,5,6,7-tetrahydro- |
| | 1H-indazol-1-yl]phenyl}methyl)-2-propanesulfonamide |
| WO 2007/107539 | N-({4[3-(trifluoromethyl)-4,5,6,7-tetrahydro- |
| | 1H-indazol-1-yl]phenyl}methyl)cyclopropanesulfonamide |
| WO 2007/107539 | N-({4[3-(trifluoromethyl)-4,5,6,7-tetrahydro-1H- |
| | indazol-1-yl]phenyl}methyl)cyclopentanesulfonamide |
| WO 2007/107539 | 1-[4-(1-pyrrolidinylsulfonyl)phenyl]-3- |
| | (trifluoromethyl)-4,5,6,7-tetrahydro-1H-indazole |
| WO 2007/107539 | N-(2-methylpropyl)-4[3-(trifluoromethyl)-4,5,6,7- |
| | tetrahydro-1H-indazol-1-yl]benzenesulfonamide |
| WO 2007/107539 | 1-[4-(4-morpholinylsulfonyl)phenyl]-3- |
| | (trifluoromethyl)-4,5,6,7-tetrahydro-1H-indazole |
| WO 2007/107539 | N-[2-(methyloxy)ethyl]-4-[3-(trifluoromethyl)- |
| | 4,5,6,7-tetrahydro-1H-indazol-1-yl]benzenesulfonamide |
| WO 2007/107539 | N-[2-(1-pyrrolidiny)ethyl]-4-[3-(trifluoromethyl)- |
| | 4,5,6,7-tetrahydro-1H-indazol-1-yl]benzenesulfonamide |
| WO 2007/107539 | N-(tetrahydro-2-furanylmethyl)-4-[3-(trifluoromethyl)- |
| | 4,5,6,7-tetrahydro-1H-indazol-1-yl]benzenesulfonamide |
| WO 2007/107539 | 1-[4-(1H-imidazol-1-ylmethyl)phenyl]-3- |
| | (trifluoromethyl)-4,5,6,7-tetrahydro-1H-indazole |
| WO 2007/107539 | 1-[4-(1H-1,2,4-triazol-1-ylmethyl)pheny1]-3- |
| | (trifluoromethyl)-4,5,6,7-tetrahydro-1H-indazole |
| WO 2007/107539 | 1-[4-(1H-pyrazol-1-ylmethyl)phenyl]-3- |
| | (trifluoromethyl)-4,5,6,7-tetrahydro-1H-indazole |
| WO 2007/107539 | 1-[4-(1H-1,2,3-triazol-1-ylmethyl)phenyl]-3- |
| | (trifluoromethyl)-4,5,6,7-tetrahydro-1H-indazole |
| WO 2007/107539 | 1-[4-(2H-1,2,3-triazol-2-ylmethyl)phenyl]-3- |
| | (trifluoromethyl)-4,5,6,7-tetrahydro-1H-indazole |
| WO 2007/107539 | 1-{4-[(4-methyl-1H-pyrazol-1- |
| | yl)methyl]phenyl}-3-(trifluoromethyl)- |
| | 4,5,6,7-tetrahydro-1H-indazole |
| WO 2007/107539 | 1-{4-[(3,5-dimethyl-1H-pyrazol-1- |
| | yl)methyl]phenyl}-3-(trifluoromethyl)- |
| | 4,5,6,7-tetrahydro1H-indazole |
| WO 2007/107539 | 3-(trifluoromethyl)-1-(4-{[3-(trifluoromethyl)- |
| | 1H-pyrazol-1-yl]methyllphenyl)-4,5,6,7-tetrahydro- |
| | 1H-indazole |
| WO 2007/107539 | 3-(trifluoromethyl)-1-(4-{[5-(trifluoromethyl)- |
| | 1H-pyrazol-1-yl]methyllphenyl)-4,5,6,7-tetrahydro- |
| | 1H-indazole |
| WO 2007/107539 | 1-(4-{[5-methyl-3-(trifluoromethyl)-1H-pyrazol- |
| | 1-yl]methyllphenyl)-3-(trifluoromethyl)-4,5,6,7- |
| | tetrahydro-1H-indazole |
| WO 2007/107539 | 1-(4-{[3-methyl-5-(trifluoromethyl)-1H-pyrazol- |
| | 1-yl]methyllphenyl)-3-(trifluoromethyl)-4,5,6,7- |
| | tetrahydro-1H-indazole |
| WO 2007/107539 | 1-{4-[(2-methyl-1H-imidazol-1-yl)methyl]phenyl}- |
| | 3-(trifluoromethyl)-4,5,6,7-tetrahydro-1H-indazole |
| WO 2007/107539 | 1-(4-{[2-(1-methylethyl)-1H-imidazol-1- |
| | yl]methyllphenyl)-3-(trifluoromethyl)-4,5,6,7- |
| | tetrahydro-1H-indazole |
| WO 2007/107539 | 1-{4-[(4-phenyl-1H-imidazol-1-yl)methyl]phenyl}- |
| | 3-(trifluoromethyl)-4,5,6,7-tetrahydro-1H-indazole |
| WO 2007/107539 | 1-{4-[(4-bromo-1H-pyrazol-1-yl)methyl]phenyl}- |
| | 3-(trifluoromethyl)-4,5,6,7-tetrahydro-1H-indazole |
| WO 2007/107539 | N-methyl-1H-imidazol-2-yl){4-[3-(trifluoromethyl)- |
| | 4,5,6,7-tetrahydro-1H-indazol-1-yl]phenyl}methanone |
| WO 2007/107539 | N-methyl-N-{4[3-(trifluoromethyl)-4,5,6,7-tetrahydro- |
| | 1H-indazol-1-yl]phenyl}-1-pyrrolidinecarboxamide |

Additional AMPA receptor potentiators can be identified using routine methods known to those skilled in the art. These methods can involve a variety of accepted tests to determine whether a given candidate compound is an upmodulator of the AMPA receptor. One illustrative assay is measurement of enlargement of the excitatory postsynaptic potential (EPSP) in in vitro brain slices, such as rat hippocampal brain slices, in response to administration of the compound of interest.

Typically in screens of this kind, slices of hippocampus from a mammal such as a rat are prepared and maintained in an interface chamber using conventional methods. For example, field EPSPs are recorded in the stratum radiatum of region CA1b and elicited by single stimulation pulses delivered once per 20 seconds to a bipolar electrode positioned in the Schaffer-commissural projections (see, e.g., Granger (1993) Synapse15: 326-329; Staubli et al. (1994) Proc. Natl. Acad. Sci., USA, 91: 777-781; Staubli et al. (1994) Proc. Natl. Acad. Sci., USA, 91: 11158-11162).

In such assays, the waveform of a normal EPSP is typically comprised of: (a) an AMPA receptor component, that has a relatively rapid rise time in the depolarizing direction and which decays within about 20 msec; (b) an NMDA receptor component that has slow rise and decay times (the NMDA portion is typically small in normal media, because the NMDA receptor channel is blocked at resting membrane potential); (c) a GABA component in the opposite (hyperpolarizing) direction as the glutamatergic (AMPA and NMDA) components, exhibiting a time course with a rise time of about 10-20 msec and very slow decay (typically about 50-100 msec or more).

The different components can be separately measured to assay the effect of a putative AMPA receptor-enhancing agent. This can be accomplished by adding agents that block the unwanted components so that the remaining detectable responses are mediated by a single class of transmitter receptor (i.e., AMPA receptors only, or NMDA receptors only, or GABA receptors only). For example, to measure AMPA responses, an NMDA receptor blocker (e.g., AP-5 or other NMDA blockers known in the art) and/or a GABA blocker (e.g., picrotoxin or other GABA blockers known in the art) are added to the slice.

AMPA receptor potentiators useful in the methods described herein include substances that cause an increased ion flux through the AMPA receptor complex channels in response to release of glutamate. Increased ion flux is typically measured as one or more of the following non-limiting parameters: at least a 10% increase in the initial slope, amplitude, decay time, or the area under the curve of the post-synaptic response elicited by stimulation of presynaptic axons and recorded at synapses known to use glutamate as a transmitter. The response can be measured with intracellular recording (whole cell clamp method or sharp electrode method) from the post-synaptic neuron on which the stimulated synapses are formed or by extracellular recording using electrodes placed in proximity to the stimulated synapses. The post-synaptic response can be measured as current influx into the post-synaptic neuron (referred to as the Excitatory Post-Synaptic Current or ‘EPSC’) or as a change in the membrane voltage of the post-synaptic neuron (referred to as the Excitatory Post-Synaptic Potential or ‘EPSP’) or as a field potential generated by the activated synapses (referred to as the field EPSP). These measurements can be readily collected in brain slices, typically taken from the hippocampus of a rat, treated to block NMDA and GABA receptors.

Another assay utilizes excised patches, e.g., membrane patches excised from cultured hippocampal slices (see, e.g., Arai et al. (1994) Brain Res. 638: 343-346. Outside-out patches are obtained from pyramidal hippocampal neurons and transferred to a recording chamber. Glutamate pulses are applied in order to elicit excitatory currents, and data are collected with a patch clamp amplifier and digitized (Arai et al. (19994) supra and Arai et al. (1996) Neurosci., 25: 573-585).

While, in certain embodiments, the membrane patches contain only glutamatergic receptors, any GABAergic currents or NMDA currents can be blocked as above (e.g., with picrotoxin and AP-5).

Certain AMPA receptor potentiators to be used in the present invention are capable of entering the brain and possess the potency and metabolic stability needed to increase synaptic responses in living animals. The central action of a drug can be verified by measurement of monosynaptic field EPSPs in behaving animals (see, e.g., Staubli et al. (1994) Proc. Natl. Acad. Sci., USA, 91: 777-781) and time course of biodistribution can be ascertained via injection and PET measurement of appropriately radiolabeled (C-11 or F-18) drug (see, e.g.,; Staubli et al. (1994) Proc. Natl. Acad. Sci., USA, 91: 11158-11162).

Gene Based Approaches.

In certain embodiments, BNDF levels are increased by transducing/transforming the subject with an expression vector encoding proBDNF, BDNF and/or a BNDF fragment or mutant that shows BDNF activity and/or by grafting cells expressing such a vector into the host. Such expression vectors include, but are not limited to, eukaryotic vectors, prokaryotic vectors (such as, for example, bacterial vectors), and viral vectors. In certain embodiments, the polynucleotide encoding the BDNF, proBDNF, and/or BDNF fragment or mutant (i.e., the BDNF transgene), or an expression vehicle containing the polynucleotide, is contained within a liposome or other delivery/transfection reagent.

Without being bound to a particular theory, it is believed that expression of the BNDF transgene in a subject showing cognitive deficit without substantial neural degeneration will improve cognitive performance, or in subjects at risk for such cognitive deficit, to reduce or prevent substantial decrease in cognitive function.

Many approaches for introducing nucleic acids into cells in vivo, ex vivo and in vitro are known to those of skill in the art. These include, but are not limited to lipid or liposome based gene delivery (see, e.g., WO 96/18372; WO 93/24640; Mannino and Gould-Fogerite (1988) BioTechniques 6(7): 682-691; Rose U.S. Pat. No. 5,279,833; WO 91/06309; and Felgner et al. (1987) Proc. Natl. Acad. Sci. USA 84: 7413-7414), electroporation, calcium phosphate transfection, viral vectors, biolistics, microinjection, dendrimer conjugation, and the like. In certain embodiments, transfection is by means of replication-defective retroviral vectors (see, e.g., Miller et al. (1990) Mol. Cell. Biol. 10:4239 (1990); Kolberg (1992) J. NIH Res. 4: 43, and Cornetta et al. (1991) Hum. Gene Ther. 2: 215).

For a review of gene therapy procedures, see, e.g., Anderson (1992) Science 256: 808-813; Nabel and Felgner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 1149-1154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Bohm (eds) Springer-Verlag, Heidelberg Germany; and Yu et al., (1994) Gene Therapy, 1: 13-26.

Widely used vectors include those based upon murine leukemia virus (MuLV), gibbon ape leukemia virus (GaLV), Simian Immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like).

The construction and use of various gene therapy vectors is also described in U.S. Pat. No. 7,074,772, U.S. Pat. No. 7,064,111, U.S. Pat. No. 7,052,881, U.S. Pat. No. 7,037,716, RE39,078, U.S. Pat. No. 7,022,319, U.S. Pat. No. 7,018,826, U.S. Pat. No. 7,001,760, and the like which are incorporated herein by reference.

The vectors are optionally pseudotyped to extend the host range of the vector to cells which are not infected by the retrovirus corresponding to the vector. For example, the vesicular stomatitis virus envelope glycoprotein (VSV-G) has been used to construct VSV-G-pseudotyped HIV vectors which can infect hematopoietic stem cells (Naldini et al. (1996) Science 272:263, and Akkina et al. (1996) J Virol 70:2581).

Adeno-associated virus (AAV)-based vectors are also used to transduce cells with target nucleic acids, e.g., in the in vitro production of nucleic acids and peptides, and in in vivo and ex vivo gene therapy procedures. See, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Construction of recombinant AAV vectors are described in a number of publications, including Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996. Other suitable viral vectors include herpes virus, lentivirus, and vaccinia virus.

In certain embodiments retroviruses (e.g. lentiviruses) are used to transfect the target cell(s) with nucleic acids encoding the BDNF transgene. Retroviruses, in particular lentiviruses (e.g. HIV, SIV, etc.) are particularly well suited for this application because they are capable of infecting a non-dividing cell. Methods of using retroviruses for nucleic acid transfection are known to those of skill in the art (see, e.g., U.S. Pat. No. 6,013,576).

Retroviruses are RNA viruses wherein the viral genome is RNA. When a host cell is infected with a retrovirus, the genomic RNA is reverse transcribed into a DNA intermediate which is integrated very efficiently into the chromosomal DNA of infected cells. This integrated DNA intermediate is referred to as a provirus. Transcription of the provirus and assembly into infectious virus occurs in the presence of an appropriate helper virus or in a cell line containing appropriate sequences enabling encapsidation without coincident production of a contaminating helper virus. In preferred embodiments, a helper virus need not be utilized for the production of the recombinant retrovirus since the sequences for encapsidation can be provided by co-transfection with appropriate vectors.

The retroviral genome and the proviral DNA have three genes: the gag, the pol, and the env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication. Lentiviruses have additional genes including vit, vpr, tat, rev, vpu, nef, and vpx (in HIV-1, HIV-2 and/or SIV).

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the result is a cis defect which prevents encapsidation of genomic RNA. However, the resulting mutant is still capable of directing the synthesis of all virion proteins.

In certain embodiments the invention provides a recombinant retrovirus capable of infecting a non-dividing cell. The recombinant retrovirus comprises a viral GAG, a viral POL, a viral ENV, a heterologous nucleic acid sequence operably linked to a regulatory nucleic acid sequence, and cis-acting nucleic acid sequences necessary for packaging, reverse transcription and integration, as described above. It should be understood that the recombinant retrovirus of the invention is capable of infecting dividing cells as well as non-dividing cells.

In preferred embodiments, the recombinant retrovirus is therefore genetically modified in such a way that some of the structural, infectious genes of the native virus (e.g. env, gag, pol) have been removed and replaced instead with a nucleic acid sequence to be delivered to a target non-dividing cell (e.g., a sequence encoding the reporter and/or cytotoxic gene under control of the HPV promoter). After infection of a cell by the virus, the virus injects its nucleic acid into the cell and the retrovirus genetic material can, optionally, integrate into the host cell genome. Methods of making and using lentiviral vectors are discussed in detail in U.S. Pat. Nos. 6,013,516, 5,932,467, and the like.

In certain embodiments, the nucleic acid encoding the BDNF, BDNF fragment or BNDM mutein(s) are placed in an adenoviral vector suitable for gene therapy. The use of adenoviral vectors is described in detail in WO 96/25507. Particularly preferred adenoviral vectors are described by Wills et al. (1994) Hum. Gene Therap. 5: 1079-1088. Typically, adenoviral vectors contain a deletion in the adenovirus early region 3 and/or early region 4 and this deletion may include a deletion of some, or all, of the protein IX gene. In one embodiment, the adenoviral vectors include deletions of the E1a and/or E1b sequences.

A number of different adenoviral vectors have been optimized for gene transfer. One such adenoviral vector is described in U.S. Pat. No. 6,020,191. This vector comprises a CMV promoter to which a transgene may be operably linked and further contains an E1 deletion and a partial deletion of 1.6 kb from the E3 region. This is a replication defective vector containing a deletion in the E1 region into which a transgene (e.g. the β subunit gene) and its expression control sequences can be inserted, preferably the CMV promoter contained in this vector. It further contains the wild-type adenovirus E2 and E4 regions. The vector contains a deletion in the E3 region which encompasses 1549 nucleotides from adenovirus nucleotides 29292 to 30840 (Roberts et al. (1986) Adenovirus DNA, in Developments in Molecular Virology, W. Doerfler, ed., 8: 1-51). These modifications to the E3 region in the vector result in the following: (a) all the downstream splice acceptor sites in the E3 region are deleted and only mRNA a would be synthesized from the E3 promoter (Tollefson et al. (1996) J, Virol. 70:2 296-2306, 1996; Tollefson et al. (1996) Virology 220: 152-162,); (b) the E3A poly A site has been deleted, but the E3B poly A site has been retained; (c) the E3 gp19K (MHC I binding protein) gene has been retained; and (d) the E3 11.6K (Ad death protein) gene has been deleted.

Such adenoviral vectors can utilize adenovirus genomic sequences from any adenovirus serotypes, including but not limited to, adenovirus serotypes 2, 5, and all other preferably non-oncogenic serotypes.

Alone, or in combination with viral vectors, a number of non-viral vectors are also useful for transfecting cells with reporter and/or cytotoxic genes under control of the HPV promoter. Suitable non-viral vectors include, but are not limited to, plasmids, cosmids, phagemids, liposomes, water-oil emulsions, polethylene imines, biolistic pellets/beads, and dendrimers.

Cationic liposomes are positively charged liposomes that interact with the negatively charged DNA molecules to form a stable complex. Cationic liposomes typically consist of a positively charged lipid and a co-lipid. Commonly used co-lipids include dioleoyl phosphatidylethanolamine (DOPE) or dioleoyl phosphatidylcholine (DOPC). Co-lipids, also called helper lipids, are in most cases required for stabilization of liposome complex. A variety of positively charged lipid formulations are commercially available and many others are under development. Two of the most frequently cited cationic lipids are lipofectamine and lipofectin. Lipofectin is a commercially available cationic lipid first reported by Phil Felgner in 1987 to deliver genes to cells in culture. Lipofectin is a mixture of N-[1-(2,3-dioleyloyx) propyl]-N-N-N-trimethyl ammonia chloride (DOTMA) and DOPE.

DNA and lipofectin or lipofectamine interact spontaneously to form complexes that have a 100% loading efficiency. In other words, essentially all of the DNA is complexed with the lipid, provided enough lipid is available. It is assumed that the negative charge of the DNA molecule interacts with the positively charged groups of the DOTMA. The lipid:DNA ratio and overall lipid concentrations used in forming these complexes are extremely important for efficient gene transfer and vary with application. Lipofectin has been used to deliver linear DNA, plasmid DNA, and RNA to a variety of cells in culture. Shortly after its introduction, it was shown that lipofectin could be used to deliver genes in vivo. Following intravenous administration of lipofectin-DNA complexes, both the lung and liver showed marked affinity for uptake of these complexes and transgene expression. Injection of these complexes into other tissues has had varying results and, for the most part, are much less efficient than lipofectin-mediated gene transfer into either the lung or the liver.

PH-sensitive, or negatively-charged liposomes, entrap DNA rather than complex with it. Since both the DNA and the lipid are similarly charged, repulsion rather than complex formation occurs. Yet, some DNA does manage to get entrapped within the aqueous interior of these liposomes. In some cases, these liposomes are destabilized by low pH and hence the term pH-sensitive. To date, cationic liposomes have been much more efficient at gene delivery both in vivo and in vitro than pH-sensitive liposomes. pH-sensitive liposomes have the potential to be much more efficient at in vivo DNA delivery than their cationic counterparts and should be able to do so with reduced toxicity and interference from serum protein.

The therapeutic potential for liposome-mediated gene transfer in the CNS has been successfully demonstrated using rodent models. Based on existing evidence which shows that the systemic injection of cDNA:cationic liposome complexes into animals is non-toxic (Stewart et al. (19992) Human Gene Ther., 3: 267-275) liposome-mediated gene transfer methods have been developed for use with neural tissue (see, e.g., U.S. Pat. No. 6,096,716; and Holt et al. (1990) Neuron, 4: 203-214).

In another approach dendrimers complexed to the DNA have been used to transfect cells. Such dendrimers include, but are not limited to, “starburst” dendrimers and various dendrimer polycations.

Dendrimer polycations are three dimensional, highly ordered oligomeric and/or polymeric compounds typically formed on a core molecule or designated initiator by reiterative reaction sequences adding the oligomers and/or polymers and providing an outer surface that is positively changed. These dendrimers may be prepared as disclosed in PCT/US83/02052, and U.S. Pat. Nos. 4,507,466, 4,558,120, 4,568,737, 4,587,329, 4,631,337, 4,694,064, 4,713,975, 4,737,550, 4,871,779, 4,857,599.

Typically, the dendrimer polycations comprise a core molecule upon which polymers are added. The polymers may be oligomers or polymers which comprise terminal groups capable of acquiring a positive charge. Suitable core molecules comprise at least two reactive residues which can be utilized for the binding of the core molecule to the oligomers and/or polymers. Examples of the reactive residues are hydroxyl, ester, amino, imino, imido, halide, carboxyl, carboxyhalide maleimide, dithiopyridyl, and sulfhydryl, among others. Preferred core molecules are ammonia, tris-(2-aminoethyl)amine, lysine, ornithine, pentaerythritol and ethylenediamine, among others. Combinations of these residues are also suitable as are other reactive residues.

Oligomers and polymers suitable for the preparation of the dendrimer polycations of the invention are pharmaceutically-acceptable oligomers and/or polymers that are well accepted in the body. Examples of these are polyamidoamines derived from the reaction of an alkyl ester of an α,β-ethylenically unsaturated carboxylic acid or an α,β-ethylenically unsaturated amide and an alkylene polyamine or a polyalkylene polyamine, among others. Preferred are methyl acrylate and ethylenediamine. The polymer is preferably covalently bound to the core molecule.

The terminal groups that may be attached to the oligomers and/or polymers should be capable of acquiring a positive charge. Examples of these are azoles and primary, secondary, tertiary and quaternary aliphatic and aromatic amines and azoles, which may be substituted with S or O, guanidinium, and combinations thereof. The terminal cationic groups are preferably attached in a covalent manner to the oligomers and/or polymers. Preferred terminal cationic groups are amines and guanidinium. However, others may also be utilized. The terminal cationic groups may be present in a proportion of about 10 to 100% of all terminal groups of the oligomer and/or polymer, and more preferably about 50 to 100%.

The dendrimer polycation may also comprise 0 to about 90% terminal reactive residues other than the cationic groups. Suitable terminal reactive residues other than the terminal cationic groups are hydroxyl, cyano, carboxyl, sulfhydryl, amide and thioether, among others, and combinations thereof. However others may also be utilized.

The dendrimer polycation is generally and preferably non-covalently associated with the polynucleotide. This permits an easy disassociation or disassembling of the composition once it is delivered into the cell. Typical dendrimer polycations suitable for use herein have a molecular weight ranging from about 2,000 to 1,000,000 Da, and more preferably about 5,000 to 500,000 Da. However, other molecule weights are also suitable. Preferred dendrimer polycations have a hydrodynamic radius of about 11 to 60 Å, and more preferably about 15 to 55 Å. Other sizes, however, are also suitable. Methods for the preparation and use of dendrimers in gene therapy are well known to those of skill in the art and describe in detail, for example, in U.S. Pat. No. 5,661,025.

Where appropriate, two or more types of vectors can be used together. For example, a plasmid vector may be used in conjunction with liposomes. In the case of non-viral vectors, nucleic acid may be incorporated into the non-viral vectors by any suitable means known in the art. For plasmids, this typically involves ligating the construct into a suitable restriction site. For vectors such as liposomes, water-oil emulsions, polyethylene amines and dendrimers, the vector and construct may be associated by mixing under suitable conditions known in the art.

Vectors (e.g., retroviruses, adenoviruses, liposomes, etc.) containing therapeutic nucleic acids can be administered directly to the organism for transduction of cells in vivo. Administration is by any of the routes normally used for introducing a molecule into ultimate contact with blood or tissue cells. The nucleic acids are administered in any suitable manner, preferably with pharmaceutically acceptable carriers. Suitable methods of administering such packaged nucleic acids are available and well known to those of skill in the art.

For example, in certain embodiments, introduction of, e.g., a liposome-cDNA transfection complex can be by injection, and can be systemic injections into peripheral arteries or veins, including the carotid or jugular vessels. Injection can also be directly into the central nervous system, either by intraventricular administration, or directly into the brain tissue itself. Such injection may be facilitated by the use of mini-osmotic pumps for long-duration infusion, or an intraparenchymal injection apparatus with ventricular cannuli or other intraparenchymal devices. In certain embodiments, it may be desirable to introduce the therapeutic agent(s), e.g., liposome-cDNA complex directly into the spinal cord or surrounding epidural space. In certain embodiments such injection may be made into the ventricle, the hippocampus, the cortex, or directly into the spinal cord.

Cell-Based Therapies.

In certain embodiments stem cells, or graft cells engineered to express BNDF, BDNF fragments, or BDNF muteins can be used to effectively increase BDNF levels in the brain.

The choice of the donor cells for implantation depends on the nature of the expressed gene (e.g., BDNF), characteristics of the vector, and the desired phenotypic result. For example, retroviral vectors require cell division and DNA synthesis for efficient infection, integration and gene expression. Thus, for the use of such vectors the donor cells are preferably actively growing cells, such as primary fibroblast culture or established cell lines, replicating embryonic neuronal cells, or replicating adult neuronal cells from selected areas such as the olfactory mucosa and possibly developing or reactive glia.

In certain embodiments primary cells, i.e. cells that have been freshly obtained from a subject, such as fibroblasts, that are not in the transformed state are used in the present invention. Other suitable donor cells include immortalized (transformed cells that continue to divide) fibroblasts, glial cells, adrenal cells, hippocampal cells, keratinocytes, hepatocytes, connective tissue cells, ependymal cells, bone marrow cells, stem cells, leukocytes, chromaffin cells and other mammalian cells susceptible to genetic manipulation and grafting. Additional characteristics of donor cells which are relevant to successful grafting include the age of the donor cells.

Furthermore, there are available methods to induce a state of susceptibility in stationary, non-replicating target cells that will allow many other cell types to be suitable targets for viral transduction. For instance, methods have been developed that permit the successful viral vector infection of primary cultures of adult rat hepatocytes, ordinarily refractory to infection with such vectors, and similar methods may be helpful for a number of other cells (Wolff et al. (1987) Proc. Natl. Acad. Sci., USA, 86:9011-9014, 1987). In addition, the development of many other kinds of vectors derived from herpes, vaccinia, adenovirus, or other viruses, as well as the use of efficient non-viral methods for introducing DNA into donor cells such as electroporation lipofection or direct gene insertion may be used for gene transfer into many other cells.

The donor cells are prepared for grafting, e.g., for injection of genetically modified donor cells, fibroblasts obtained from for example, skin samples are placed in a suitable culture medium for growth and maintenance of the cells. In certain embodiments such a solution may contain fetal calf serum (FCS) in which the cells are allowed to grow to confluence. The cells are loosened from the culture substrate for example using a buffered solution containing 0.05% trypsin and placed in a buffered solution such as PBS supplemented with 5% serum to inactivate trypsin. The cells may be washed with PBS using centrifugation and then resuspended in the complete PBS without trypsin and at a selected density for injection. In addition to PBS, any osmotically balanced solution which is physiologically compatible with the host subject may be used to suspend and inject the donor cells into the host.

The long-term survival of implanted cells may depend on the mode of transfection, on cellular damage produced by the culture conditions, on the mechanics of cell implantation, or the establishment of adequate vascularization, and on the immune response of the host animal to the foreign cells or to the introduced gene product. The mammalian brain has traditionally been considered to be an immunologically privileged organ, but recent work has shown conclusively that immune responses can be demonstrated to foreign antigens in the rat brain. The potential for rejection and graft-versus-host reaction induced by the grafted cells is reduced by using autologous cells wherever feasible, and by the use of vectors that will not produce changes in cell surface antigens other than those associated with the phenotypic correction and possibly by the introduction of the cells during a phase of immune tolerance of the host animal, as in fetal life.

The most effective mode and timing of grafting of the transgene donor cells will depend on the severity of the defect and on the severity and course of pathology and response to treatment and the judgment of the treating health professional.

The methods of the invention contemplate intracerebral grafting of donor cells containing a transgene insert (e.g., expressing proBNDF, BDNF, or a BNDF fragment or mutant having BDNF activity) in to the brain. Neural transplantation or “grafting” involves transplantation of cells into the central nervous system or into the ventricular cavities or subdurally onto the surface of a host brain. Conditions for successful transplantation typically include: 1) viability of the implant; 2) retention of the graft at the site of transplantation; and 3) minimum amount of pathological reaction at the site of transplantation.

Methods for transplanting various nerve tissues, for example embryonic brain tissue, into host brains have been described in Neural Grafting in the Mammalian CNS, Bjorklund and Stenevi, eds., (1985) Das, Ch. 3 pp. 23 30; Freed, Ch 4, pp. 31 40; Stenevi et al., Ch. 5, pp. 41 50; Brundin et al., Ch. 6, pp. 51 60; David et al., Ch. 7, pp. 61 70; Seiger, Ch. 8, pp. 71 77 (1985), incorporated by reference herein. These procedures include intraparenchymal transplantation, i.e. within the host brain (as compared to outside the brain or extraparenchymal transplantation) achieved by injection or deposition of tissue within the host brain so as to be opposed to the brain parenchyma at the time of transplantation (Bjorklund and Stenevi, eds., (1985) Ch.3, pp. 23-30 in Neural Grafting in the Mammalian CNS).

Two common procedures for intraparenchymal transplantation include: 1) injecting the donor cells within the host brain parenchyma or 2) preparing a cavity by surgical means to expose the host brain parenchyma and then depositing the graft into the cavity. Both methods provide parenchymal apposition between the graft and host brain tissue at the time of grafting, and both facilitate anatomical integration between the graft and host brain tissue.

In certain alternative approaches, the graft can be placed in a ventricle, e.g. a cerebral ventricle or subdurally, e.g., on the surface of the host brain where it is separated from the host brain parenchyma by the intervening pia mater or arachnoid and pia mater. Grafting to the ventricle can be accomplished by injection of the donor cells or by growing the cells in a suitable substrate (e.g., 3% collagen) to form a plug of solid tissue which can then be implanted into the ventricle to prevent dislocation of the graft. For subdural grafting, the cells can be injected around the surface of the brain, e.g., after making a slit in the dura. Injections into selected regions of the host brain can be made by drilling a hole and piercing the dura to permit the needle of a microsyringe to be inserted. The microsyringe can be mounted in a stereotaxic frame and three dimensional stereotaxic coordinates can selected for placing the needle into the desired location of the brain or spinal cord.

The donor cells may also be introduced into the putamen, nucleus basalis, hippocampus cortex, striatum or caudate regions of the brain, as well as, in certain embodiments, the spinal cord.

The cellular suspension procedure thus permits grafting of genetically modified donor cells to any predetermined site in the brain or spinal cord, is relatively non-traumatic, allows multiple grafting simultaneously in several different sites or the same site using the same cell suspension, and permits mixtures of cells from different anatomical regions. Multiple grafts may consist of a mixture of cell types, and/or a mixture of transgenes inserted into the cells. In certain embodiments from approximately 10⁴ to approximately 10¹² cells are introduced per graft. Thus it is envisioned that in certain embodiments, 10⁵, 10⁶, 10⁷, 10⁸, 10⁸ 10¹⁰ or 10¹¹ cells may be introduced per graft. Additionally it is contemplated that more than one graft may be necessary, indeed 1, 2, 3, 4, 5, 6, 7, 8, 9, 10 or more grafts may be performed over any given period ranging from days to weeks to months to years.

The methods of the invention also contemplate the use of grafting of transgenic donor cells in combination with other therapeutic procedures to treat disease or trauma in the CNS. Thus, genetically modified donor cells of the invention may be co-grafted with other cells, both genetically modified and non-genetically modified cells which exert beneficial effects on cells in the CNS, such as chromaffin cells from the adrenal gland, fetal brain tissue cells and placental cells. The genetically modified donor cells may thus be supported by the survival and function of co-grafted, non-genetically modified cells.

Other Approaches.

Other compounds for use in the methods of this invention include compounds that mimic the effects of BDNF. Such compounds include, but are not limited to peptides that are monocyclic and bicyclic loop mimetics of the neurotrophin. Furthermore, neurohormones (e.g. estrogen, adrenocorticotropin) and neurotransmitters and their precursors (e.g. dopamine, norepinephrine, LDOPA, serotonin) can up-regulate BDNF as well as compounds that mimic or increase levels of these neurochemicals (e.g. Semax is an analogue of the neurohormone adrenocorticotropin that increases BDNF levels). Finally, compounds that increase the activity of BDNF possibly through up-regulating its receptor (e.g. kinase inhibitors) are also viable therapeutics.

Thus, various methods of increasing BDNF levels include, but are not limited to glutamate AMPA receptor modulators (e.g. ampakines) as described above, physical exercise, dietary restriction, anti-depressant drugs (e.g. fluoxetine, desipramine, 2-methyl-6-(phenylethynyl)-pyridine), anti-anxiolytics (e.g. afobazole), histone deacetylase inhibitors (e.g. sodium butyrate), neuropeptides (e.g. cocaine- and amphetamine-regulated transcript), cystamine and related agents, nicotine, anti-psychotics (e.g. quetiapine, venlafaxine), and acetylcholinesterase inhibitors (e.g. huperzine A).

In certain embodiments, however, the methods of this invention expressly exclude exercise and/or application of any one or more of the agents described above with the exception of ampakines.

### Pharmaceutical Formulations.

In various embodiments the above described compounds and/or compositions are formulated for administration to mammal (e.g. to a human in need thereof). In certain embodiments such formulation involves combining the active component with a pharmaceutically acceptable excipient.

The compounds, e.g., ampakines, can be incorporated into a variety of formulations for therapeutic administration. Thus, in certain embodiments, the compounds can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the compounds can be achieved in various ways, including oral, buccal, rectal, parenteral, intraperitoneal, intradermal, transdermal, intracheal. etc., administration. In certain embodiments preferably the therapeutic agents (e.g., ampakines) are sufficiently able to penetrate the blood-brain barrier so that their administration into the systemic circulation results in a therapeutically effective amount in the brain.

The compounds of the present invention can be administered alone, in combination with each other, or they can be used in combination with other known compounds (e.g., other memory or learning enhancing agents). In pharmaceutical dosage forms, the compounds can be administered in the form of their pharmaceutically acceptable salts, or they may also be used alone or in appropriate association, as well as in combination with other pharmaceutically active compounds. The following methods and excipients are merely illustrative and are in no way limiting.

For oral preparations, the therapeutic agents (e.g., ampakines) can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

In certain embodiments the compounds are made into suppositories by mixing with a variety of bases such as emulsifying bases or water-soluble bases. The compounds can be administered rectally via a suppository. The suppository can include vehicles such as cocoa butter, carbowaxes and polyethylene glycols, that melt at body temperature, yet are solidified at room temperature.

Unit dosage forms for oral or rectal administration such as syrups, elixirs, and suspensions can be provided wherein each dosage unit, for example, teaspoonful, tablespoonful, tablet or suppository contains a predetermined amount of the therapeutic agent. Similarly, unit dosage forms for injection or intravenous administration may comprise the compound of the present invention in a composition as a solution in sterile water, normal saline or another pharmaceutically acceptable carrier.

The term “unit dosage form” refers to physically discrete units suitable as unitary dosages for human and/or animal subjects, each unit containing a predetermined quantity active agent in an amount sufficient to produce the desired effect, optionally in association with a pharmaceutically acceptable diluent, carrier or vehicle. The specifications for the unit dosage form depends on the particular compound employed, the effect to be achieved, and the pharmacodynamics associated with each compound in the host.

Pharmaceutically acceptable excipients, such as vehicles, adjuvants, carriers diluents, pH adjusting and buffering agents, tonicity adjusting agents, stabilizers, wetting agents, and the like are known and readily available to those of skill in the art (see, e.g., Remington's Pharmaceutical Science, 15th ed., Mack Publishing Company, Easton, Pa. (1980

In certain embodiments preferred formulations of the compounds (e.g., ampakines) include oral preparations, particularly capsules, tablets, gelcaps, and the like containing each from about 1 or 10 milligrams up to about 1,000 milligrams of active ingredient. The compounds are formulated in a variety of physiologically compatible matrixes or solvents.

### Dosage

In various embodiments the above described compounds and/or compositions are administered at a dosage partially or fully mitigates, eliminates, or prevents cognitive dysfunction and/or one or more symptoms thereof in subjects having or at risk for fragile x syndrome and/or other pathologies characterized by cognitive dysfunction with little or no neural degeneration (e.g., Down's syndrome, autism, etc.).

Dosages for systemic AMPA receptor potentiators typically range from about 0.01 mg/kg to about 100 mg/kg, preferably from about 0.1 mg/kg to about 10 mg/kg, more preferably from about 0.1, or 0.5 to about 5, 2, or 1 milligrams per kg weight of subject per administration. An illustrative typical dosage may be one 5-200 mg tablet taken once a day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

Dose levels can vary as a function of the specific compound, the severity of the symptoms, and the susceptibility of the subject to side effects. Some of the specific compounds that stimulate glutamatergic receptors are more potent than others. Suitable dosages for a given compound are readily determinable by those of skill in the art by a variety of means known to those of skill in the art.

In certain embodiments AMPA receptor potentiators are typically administered together with AChE inhibiting compounds. Although the inhibitors are effective in their normal therapeutic range, compounds are preferably administered close to or at their optimal therapeutic doses. The range of therapeutically effective doses for mammalian subjects typically ranges from about 0.02 to about 0.2 mg per kilogram of body weight per day, or preferably between about 0.1 mg/kg to about 0.5 mg/kg of body weight per day, more preferably between about 10 mg/kg to about 250 mg/kg, depending on the particular AChE inhibitor administered, route of administration, dosage schedule and form, and general and specific responses to the drug.

Suitable acetylcholinesterase inhibitors include, but are not limited totacrine hydrochloride, commercially known as Cognex, donepezil hydrochloride, commercially known as Aricept, rivastigmine tartrate, commercially known as galantamine hydrobromide, commercially known as Reminyl, and the like.

For convenience, the total daily dosage may be divided and administered in portions throughout the day, if desired. The therapeutically effective dose of drugs administered to adult human patients also depends on the route of administration, the age, weight and condition of the individual. Some patients who fail to respond to one drug may respond to another, and for this reason, several drugs may have to be tried to find the one most effective for an individual patient.

Particular optimal dosages depend on the relative potency and bioavailability of the various drugs of choice. These parameters can vary by several fold depending on the drugs being considered. As a preliminary estimate of the dosages in humans, biological effects provided in rat or other animal models provide a first guide to dosing in the human recognizing that animal models are often dosed at least a 10-fold to 100-fold excess of the drug to ensure operability under laboratory conditions.

### Kits.

In another embodiment this invention provides kits for partially or fully preserving, improving, or restoring cognitive function in mammal having or at risk for cognitive impairment and/or a learning disability. In certain embodiments the kits comprise a container containing one or more agents that increase the expression, availability, and/or activity of BDNF in the brain of a subject mammal (e.g., a human having or at risk for a cognitive impairment and/or a learning disability, particularly where the mammal shows no substantial neural degeneration). In certain embodiments the agent(s) comprise an ampakine, and in certain embodiments, the ampakine is a high impact ampakine.

In certain embodiments the kits comprise a nucleic acid construct that expresses BDNF, a pro-BNDF, an active BDNF fragment, or a BDNF mutein, and/or a vector comprising such a construct, and/or a cell containing such a construct.

The kit can, optionally, further comprise one or more other agents used in the treatment of the condition/pathology of interest.

In addition, the kits optionally include labeling and/or instructional materials providing directions (i.e., protocols) for the practice of the methods or use of the “therapeutics” or “prophylactics” of this invention. Preferred instructional materials describe the use of one or more active agent(s) of this invention to partially or fully preserve, improve, or restore cognitive function in mammal having cognitive impairment and/or a learning disability (e.g., a subject having or at risk for fragile X syndrome, Down's syndrome, autism, Rett's syndrome, nonsyndromic X-linked mental retardation, etc.).

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

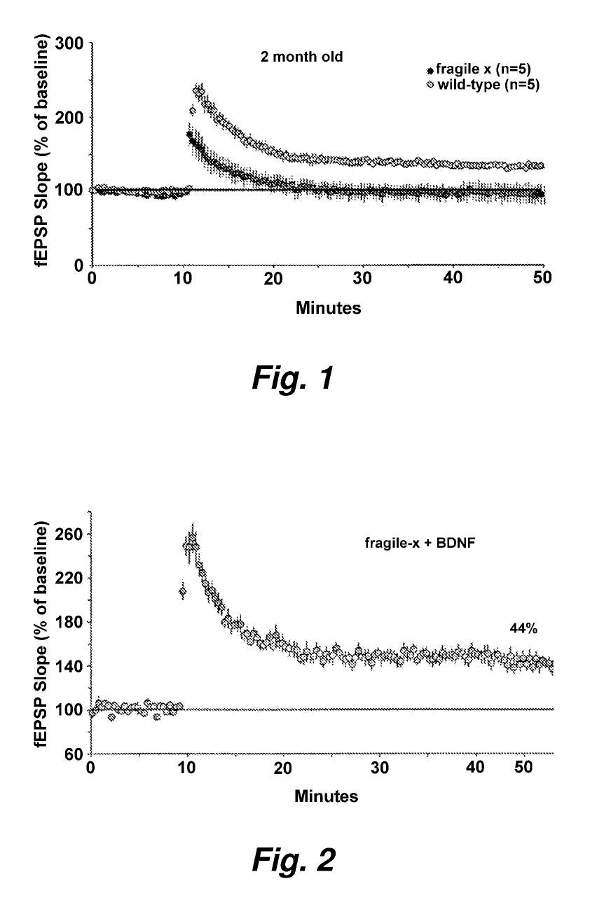

Electrophysiological studies of hippocampal slices from young adult Fmr1-KO and wildtype mice demonstrated that Fmr1-KOs had altered long term potentiation (LTP) within the apical dendritic field of region CAI. Specifically, using a sub-threshold electrical stimulation paradigm to induce LTP, hippocampal slices from fragile X mice had no detectable LTP whereas wildtype slices showed stable LTP (411% above baseline). A series of experiments in young mice (2-3 mo old) that demonstrated a similar difference between Fmr1-KOs and WTs on LTP (see, e.g., FIG. 1). We treated hippocampal slices with BDNF (15 ng/ml) and found that it restored normal LTP in the Fmr1-KO slices (>40% above baseline) (see, e.g., FIG. 2).

This finding demonstrates that BDNF can restore normal synaptic plasticity in a mouse model of fragile X and indicates that BDNF could be used to treat mental retardation. For the purposes of this invention, any and all means to increases BDNF levels in the brain could be used. Ultimately, BDNF levels need to be sufficient to facilitate LTP, thus overcoming the deficit and leading to normal cognitive function.

### Example 2

### Brain-Derived Neurotrophic Factor Rescues Synaptic Plasticity in a Mouse Model of Fragile X Syndrome

Mice lacking expression of the fragile X mental retardation 1 (Fmr1) gene have deficits in types of learning that are dependent on the hippocampus. Here, we report that long-term potentiation (LTP) elicited by threshold levels of theta burst afferent stimulation (TBS) is severely impaired in hippocampal field CA1 of young adult Fmr1 knock-out mice. The deficit was not associated with changes in postsynaptic responses to TBS, NMDA receptor activation, or levels of punctate glutamic acid decarboxylase-65/67 immunoreactivity. TBS-induced actin polymerization within dendritic spines was also normal. The LTP impairment was evident within 5 min of induction and, thus, may not be secondary to defects in activity-initiated protein synthesis. Protein levels for both brain-derived neurotrophic factor (BDNF), a neurotrophin that activates pathways involved in spine cytoskeletal reorganization, and its TrkB receptor were comparable between genotypes. BDNF infusion had no effect on baseline transmission or on postsynaptic responses to theta burst stimulation, but nonetheless fully restored LTP in slices from Fragile X mice. These results indicate that the fragile X mutation produces a highly selective impairment to LTP, possibly at a step downstream of actin filament assembly, and suggest a means for overcoming this deficit. The possibility of a pharmacological therapy based on these results is discussed.

Fragile X syndrome (FXS), a common form of inherited mental retardation, is typically caused by an expansion of CGG-repeats in the gene [fragile X mental retardation 1 (Fmr1)] that encodes fragile X mental retardation protein (FMRP); expression of the gene is blocked, and the disease appears, when the number of repeats passes a threshold length (˜200). The fragile X protein associates with polyribosomes and functions as a negative regulator of protein synthesis (Todd and Malter, 2002) including that occurring in the vicinity of dendritic spines (Zalfa et al., 2003; Weiler et al., 2004; Muddashetty et al., 2007). Fmr1-knock-out (KO) mice, developed to model the disease, breed normally, generate full knock-out progeny, and exhibit impaired learning in the Morris water maze (The Dutch-Belgian Fragile X Consortium, 1994; Oostra and Hoogeveen, 1997). Although there are no gross brain abnormalities, adult knock-out mice have unusually long, thin spines in apical dendrites of neocortical and hippocampal pyramidal neurons (Comery et al., 1997; Irwin et al., 2002; Grossman et al., 2006). Similarly, abnormal spines have been observed in autopsy material from patients with FXS (Rudelli et al., 1985; Hinton et al., 1991; Irwin et al., 2001) or other forms of mental retardation (Marin-Padilla, 1972, 1974; Lund, 1978). These findings suggest that dendritic spines, and possibly the excitatory synapses associated with them, fail to fully mature in these conditions.

Long-term potentiation (LTP), a form of synaptic plasticity implicated in the encoding of memory (Cooke and Bliss, 2006), is accompanied by changes in the cytoskeletal organization and morphology of dendritic spines (Meng et al., 2003; Lin et al., 2005a; Carlisle and Kennedy, 2005). It is thus possible that spine abnormalities found in FXS disrupt the production of LTP and thereby produce learning problems that characterize the syndrome. However, although deficits in activity-dependent synaptic plasticity are reported for cingulate (Zhao et al., 2005) and somatosensory (Desai et al., 2006) cortices as well as for conventional LTP in somatosensory (Li et al., 2002) and piriform (Larson et al., 2005) cortices of Fmr1-KO mice, there is no evidence for an impairment in the hippocampus (Godfraind et al., 1996; Paradee et al., 1999; Li et al., 2002; Larson et al., 2005). As to mechanisms underlying the cortical impairments, Meredith et al. (2007) reported aberrant calcium signaling in dendrites and spines of Fmr1-KO prefrontal cortical neurons.

Previous studies have shown that the use of intense afferent stimulation to induce LTP can mask deficits that are evident when threshold, physiologically plausible conditions are used (Lynch et al., 2007a). As might be expected from this observation, treatment with agents known to promote the induction of LTP in normal tissue can reverse age- (Rex et al., 2006) or disease-related (Lynch et al., 2007b) disturbances found with threshold-levels of stimulation. The present studies were prompted by these findings and had three objectives: (1) to determine if hippocampal LTP is impaired in Fmr1-KOs at threshold levels of stimulation; (2) to identify the causes of any such deficits; (3) to test whether impairments can be reversed by brain-derived neurotrophic factor (BDNF), a potent endogenous modulator of the potentiation effect (Bramham and Messaoudi, 2005).

### Materials and Methods

Electrophysiology.

Transverse hippocampal slices (350 m) through the mid-septotemporal hippocampus were prepared with a vibratome (VT 1000 S; Leica, Bannockbum, Ill.) in ice-cold artificial CSF [ACSF; containing (in mM) 124 NaCl, 3 KCl, 1.25 KH₂PO₄, 3.4 CaCl₂, 2.5 MgSO₄, 26 NaHCO₃, and 10 dextrose, pH 7.35) from young (2-3 months old) adult Fmr1-KO and wild-type (WT) mice (unless otherwise specified, chemicals were from Sigma, St. Louis, Mo.). Past work has shown that interface slices maintained in these cation concentrations reliably reproduce a broad array of physiological characteristics found in vivo and, in addition, exhibit excellent stability over hours of testing. Related to this, pharmacological results obtained with such slices accurately predict effects obtained with chronic recordings or biochemical assays from behaving animals (Staubli et al., 1994; Lauterbom et al., 2003), a point that is of importance to those aspects of the present work concerned with therapeutic strategies. All experiments were initiated between 9:00 and 11:00 A.M., and slices from Fmr1-KO and WT animals were randomized across two chambers and run simultaneously. Slices were maintained at 31±1° C. in an interface recording chamber with the slice surface exposed to warm, humidified 95% O₂/5% CO₂ and continuous ACSF perfused at a rate of 60-70 ml/hr. Slices equilibrated to the chamber for at least 1 h before recordings were initiated. A single glass electrode (2 M NaCl) was placed within the mid proximodistal CA1b stratum radiatum and was used to record field EPSPs (fEPSPs) from the apical dendrites of CA1 pyramidal cells. Orthodromic stimulation was delivered at two sites (CA1a and CA1c) in the apical Schaffer collateral-commissural projections to provide convergent activation of CA1b pyramidal cells. Pulses were administered in an alternating manner to the two electrodes at 0.05 Hz by using a current that elicited a 50% maximal response. Only after a stable baseline was achieved for a minimum of 10-15 min were slices stimulated for response characterization. Input-output and paired-pulse facilitation assays were performed as described previously (Rex et al., 2005). Synaptic potentiation was induced with a train of 5 or 10 theta bursts (each containing four pulses at 100 Hz, with an interburst interval of 200 ms) Carson et al., 1986; Kramar and Lynch, 2003; Rex et al., 2005). Evoked responses were digitized (NacGather 2.0; Theta Burst, Irvine, Calif.) and analyzed for amplitude and fall slope; data are presented as a percentage of baseline. Responses to individual theta bursts were analyzed to determine the burst area; to evaluate treatment effects on theta train facilitation, responses to each burst in the theta train are presented as a percentage change from the area of the initial burst response. Unless otherwise stated, group size values presented in the figures represent number of slices tested. Generally two to three slices were tested from a given mouse, and no fewer than three mice were used in any group; for statistical analyses, each slice was considered an individual n. Statistical significance was assessed using either two-way repeated-measures ANOVA to compare values that were stable over time or Mann-Whitney Utest to compare groups expressing different decay rates (i.e., values not stable over time); statistical analyses were performed using SPSS version 15.0 (SPSS, Chicago, Ill.) Variance for physiology experiments is expressed as SEM.

A recirculating perfusion system (oxygenated and heated as above) with a peristaltic pump (60 ml/hr; MasterFlex C/L; Cole-Parmer, Vernon Hills, Ill.) was used for experiments in which purified BDNF (catalog #GF029; Millipore, Temecula, Calif.) was administered to slices. The purity of the recombinant (mature) BDNF was confirmed using Western blot analyses: a single 14 kDa band was observed under denaturing conditions. Slices received BDNF for 30 min to 1 h before physiological recording. BDNF stock was prepared in ddH2O at a concentration of 50 ng/ml (this is equivalent to 1.85 nM based on the 27 kDa size of the dimer) and stored at −20° C. Control slices from the same animals received ACSF alone in parallel on a second recirculating interface chamber. As a control for the specificity of the effect of BDNF, additional experiments were conducted in which slices from the same animal were treated with either BDNF or heat-inactivated BDNF; BDNF was heat-inactivated by boiling for 5 min immediately before use.

In Situ Labeling of F-Actin and Quantification of Dendritic Spines.

Forty minutes after theta burst afferent stimulation (TBS), AlexaFluor 568-phalloidin (6 μM, 4 μL; Invitrogen, Carlsbad, Calif.) was topically applied via micropipette four times separated by 3 min, and the tissue was then immediately fixed using 4% paraformaldehyde in 0.1 M sodium phosphate buffer (PB), pH 7.2. The application of phalloidin after stable LTP precludes any possibility of disturbances to the induction and early expression of LTP (Rex et al., 2007). After overnight fixation, slices were cryoprotected with 20% sucrose in PB (1-2 h), sectioned at 20 m on a freezing microtome (parallel to broad slice face), mounted onto Super-frost Plus slides, and coverslipped with Vectashield (Vector Laboratories, Burlingame, Calif.).

Sections were examined using epifluorescence illumination on an Olympus (Center Valley, Pa.) AX70 photomicroscope and Optronics Microfire CCD camera with a 40× oil PlanApo objective (NA 1.0). Quantitative analyses were performed on three serial sections situated 20-80 m below the surface of the original slice. A series of 20-30 high-resolution digital photomicrographs were taken at 0.2 m focal steps through each section (z-stacks). Camera exposure time was adjusted for each experiment so that approximately four to eight densely labeled spines could be visualized in the sample field of control slices. Images intended for comparison were then collected with the same illumination and exposure settings. z-stacks were collapsed into a single image by extended focal imaging (Microsuite FIVE; Soft Imaging Systems, Lakewood, Colo.) converted to grayscale, and intensity levels were scaled equally across all images (Photoshop CS2 version 9; Adobe Systems, Mountain View, Calif.) to values determined for each experiment to visualize low-intensity labeling.

Labeled spine-like structures were measured and counted from a 550 m2 sampling zone in proximal stratum radiatum between the two stimulating electrodes using in-house software described in detail previously (Lin et al., 2005b; Rex et al., 2007). Briefly, intensity thresholds (8 bits/pixel) were applied to identify spine-like structures at varying levels of label intensity within a range that reliably counted spine-like puncta. Pixel values for each image were normalized to reduce the impact of background intensity differences across the image, binarized using each intensity threshold, and finally cleaned by “erosion” and “dilation” filtering (Jain, 1984). Spine counts from three serial sections were averaged to produce a representative value for each tissue slice (Rex et al., 2007). Counting was done blindly on batches of slices that had been sectioned and stained together. Digital images of objects included in the counts were overlaid semitransparently with the original photomicrographs to confirm that they were spines.

Phosphorylated-Cofilin Immunofluorescence.

Standard electrophysiological recording and delivery of TBS was performed on acute hippocampal slices (see above). Slices were collected 5-7 min after TBS and fixed and sectioned as described above. Sections from both genotypes were simultaneously processed for immunostaining using rabbit anti-pcofilin antisera (1:100; catalog #12866; Abcam, Cambridge, UK). Sections were incubated with antisera for 40 h in PB containing 4% BSA and 0.3% Triton X-100 (PBT) at 4° C. Slides were then rinsed in PB, incubated (1 h, room temperature) in AlexaFlour-594 anti-rabbit IgG (1:1000; Invitrogen) in PBT, rinsed again, and coverslipped with Vectashield (Vector Laboratories). Control sections were processed through all procedures with primary antisera omitted from the first incubation; no labeling was visualized under these conditions.

Laser scanning confocal microscopy was performed using the Bio-Rad Laboratories (Hercules, Calif.) Radiance 2000 Laser Scanning System as described previously (Chen et al., 2007). Optical sections (1.0 m) were scanned (512×512 pixel format) with a 60× objective (1.4 NA) and magnified with 4× zoom. Image montages covering a 42,025 m2 area were collected from the zone between the two stimulation electrodes containing potentiated synapses. A sample field (3,126 m2) was converted to grayscale and intensity levels were scaled to values determined for each experiment (Photoshop CS, version 8.0; Adobe Systems) to visualize low-intensity labeling. Spine measurements were performed as described previously (Chen et al., 2007). Analysis was conducted blind on cohorts of slices from Fmr1-KOs and WTs that had been sectioned and stained together. Automatic spine identification and synapse area measurements were performed as described previously (Lin et al., 2005a; Chen et al., 2007) and above. Identified objects measuring <0.04 m2 and >1.2 m2 were excluded from analysis.

GAD Immunofluorescence.

Young adult (2-3 months old) Frm1-KO and WT mice were perfused with 4% paraformaldehyde in 0.1 M PB, pH 7.2, and brains were processed for the immunocytochemical localization of glutamic acid decarboxylase (GAD) isoforms 65 and 67. Briefly, tissue was preincubated in 0.1 M PB containing 3% normal goat serum and 0.1% Triton-X for 1 h at room temperature. Tissue was then incubated with rabbit anti GAD-65/67 (catalog #AB151 1, Millipore) diluted 1:1000 in 0.1 M PB at 4° C. for 48 h, rinsed in 0.1 M PB, and then incubated in AlexaFluor 488 anti-rabbit (1:1000; Invitrogen) at room temperature for 1 h. After rinses in PB, tissue was mounted onto slides and coverslipped with Vectashield.

Widefield photomicrographs of GAD 65/67—immunolabeling in CA1 stratum radiatum were collected on a Leica (Bannockbum, Ill.) DM6000 B microscope using a 63× Plan Apo (1.4 NA) objective. Z-series (0.2 m step) images were deconvolved by iterative restoration using Volocity 4.0 Restoration software (Improvision, Lexington, Mass.). For each animal, GAD-immunoreactive puncta were counted from a 40,000 m³ sampling zone in proximal stratum radiatum in 10 adjacent tissue sections using in-house software as described above (see phalloidin analysis) with parameters set to identify puncta; analysis was conducted blind. Counts were then averaged to give an animal mean per 40,000 m³ (from a 1024×1344×3 m sample zone). Statistical significance was assessed by Student's t test using GraphPad (San Diego, Calif.) Prism Version 4.0.

Western Blotting.

Bilateral hippocampi were dissected and pooled for each animal (n=6 for each genotype). Tissue was homogenized in cold RIPA buffer (10 mM Tris, pH 7.2, 158 mM NaCl, 1 mM EDTA, 0.1% SDS, 1% sodium deoxycholate, 1% Triton-X, 1 mM Na₃VO₄, and 1× complete protease inhibitor cocktail; Roche Diagnostics, Indianapolis, Ind.). Sample protein levels were measured (Bio-Rad Protein Assay) and volumes adjusted to normalize protein content. Proteins were then separated using 15% SDS PAGE (25 g/lane), transferred to polyvinylidene difluoride membranes (Hybond-P; GE Healthcare Bio-Sciences, Piscataway, N.J.), and processed for Western blot analysis of levels of BDNF and TrkB immunoreactivity using rabbit anti-BDNF that detects both precursor and mature BDNF (N20, catalog #s.c.-546; Santa Cruz Biotechnology, Santa Cruz, Calif.) (Michalski and Fahnestock, 2003) and rabbit anti-TrkB (catalog #T14930; Transduction Laboratories, Lexington Ky.). Briefly, membranes were blocked in 5% nonfat dry milk, in Tris-buffered saline Tween 20 (TBST) for 1 hand then incubated in antisera diluted to 1:5000 for anti-BDNF or 1:2000 for anti TrkB in 5% milk/TBST for 2 hat room temperature. After 1 h incubation with HRP-conjugated anti-rabbit IgG (1:10,000; GE Healthcare Bio-Sciences) in 5% milk/TBST, immunoreactive bands were visualized by enhanced chemiluminescence using ECL-Plus kit and reagents (GE Healthcare Bio-Sciences). To control for loading differences across lanes, membranes were stripped and reprobed for actin using mouse anti-actin diluted 1:200,000 (clone AC-15; Sigma) or tubulin using mouse anti-f3-tubulin diluted 1:400,000 (catalog #T4026; Sigma). Preliminary studies demonstrated that hippocampal whole homogenate levels of actin and tubulin immunoreactivities did not vary between WT and mutant mice. Levels of immunoreactivity were assessed by densitometric analysis of films using the NIH Image 1.62 system; levels of BDNF and TrkB immunoreactivity were normalized to actin levels as assessed for the same Western blot lane. Statistical significance was determined by Student's t test using GraphPad Prism Version 4.0.

### Results

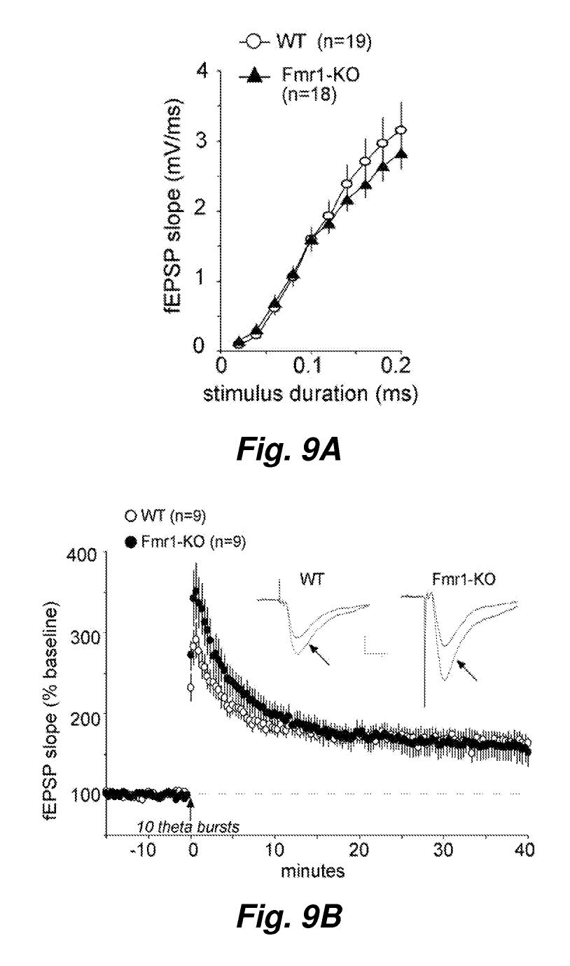

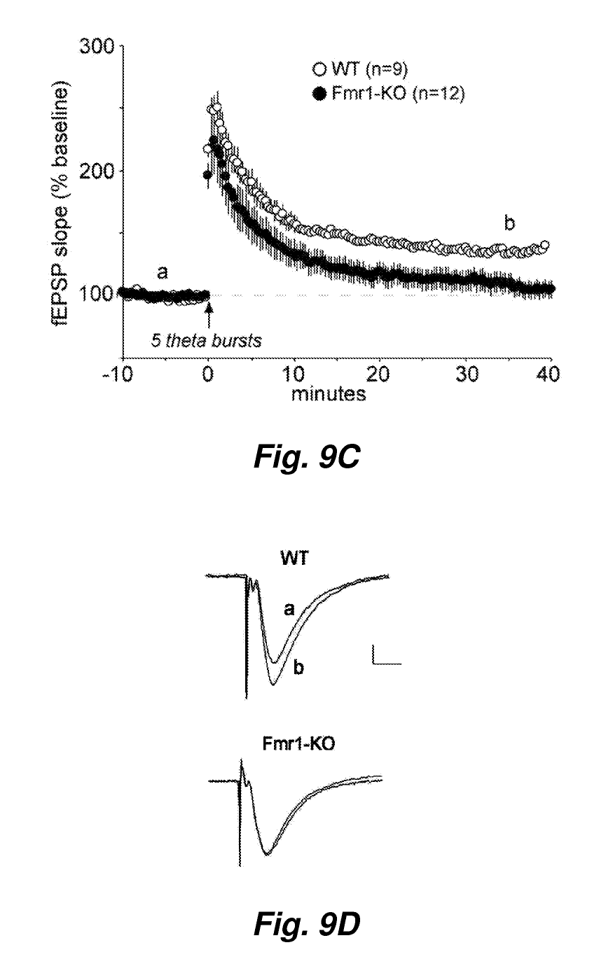

Hippocampal slices were prepared from 2-month-old Fmr1-KO and WT mice. Input-output curves for fEPSP elicited in the CA1 region by stimulation of the Schaffer commissural projections were not detectably different between the two groups (p>0.1, repeated measures ANOVA) (FIG. 9A). Potential genotype differences in presynaptic release probability were assessed by paired pulse facilitation with 50, 100, 150, and 200 ms interpulse intervals. The measure showed no effect of genotype (p>0.35 for all intervals; two-tailed t tests; n=11 and 10 for WT and Fmr1-KO, respectively). Past studies indicate that a train of 10 theta bursts is well above threshold and induces a near maximal degree of LTP in field CA1; that is, adding more bursts, or pulses to individual bursts, does not substantially affect the percentage potentiation obtained (Larson et al., 1986; Kramar et al., 2004). As shown in FIG. 9B, a single train of 10 theta bursts produced a >50% increase in the slope of fEPSPs with no evident differences between WT and mutant mice (p>0.5, two-way repeated-measures ANOVA for minutes 30-40). This confirms previous reports using different stimulation paradigms that the machinery for generating LTP in hippocampus is present in Fmr1-KO mice (Godfraind et al., 1996; Li et al., 2002). However, a different result was obtained with five theta bursts (FIG. 9C): LTP in the wildtypes (+35.3±2.3%, mean ±SEM at 35-40 min after TBS) was only slightly reduced from that found with the longer trains, whereas LTP in the mutants rapidly decayed to baseline (+7.1±7.6%). The difference between WT and Fmr1-KO slices was highly significant (p=0.012, two-way repeated measures ANOVA for minutes 30-40). Examination of Fmr1-KO responses immediately after stimulation demonstrated that initial potentiation was comparable to that in the WTs. Significant group differences were evident by 5 min post stimulation (+93.1±9.8% for WTs vs 43.0±11.6% for the KOs; p=0.011). It is likely, then, that the mutation disrupted aspects of LTP production that occur in advance of activity-driven protein synthesis thought to subserve late LTP (Reymann and Frey, 2007).

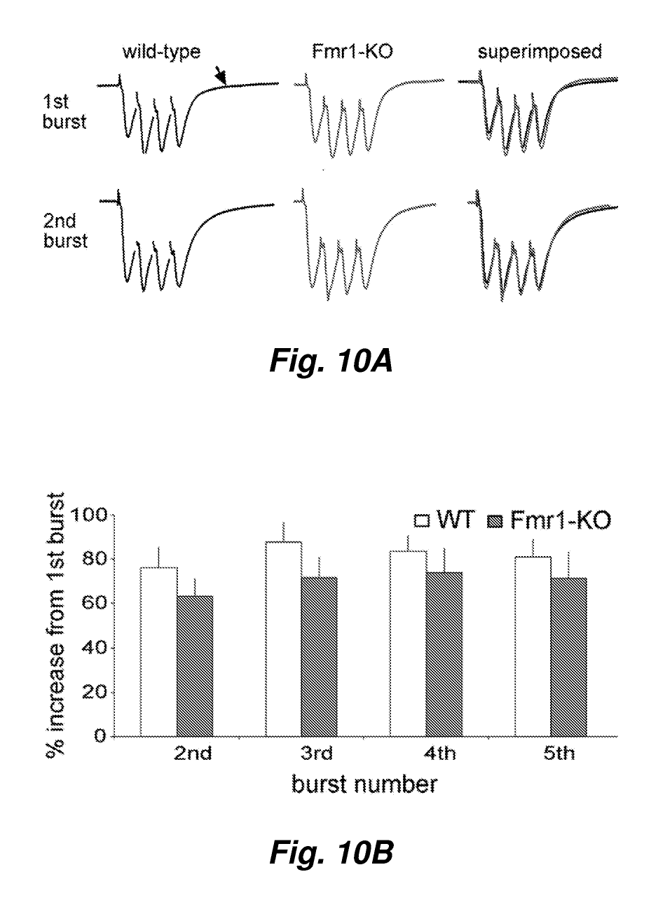

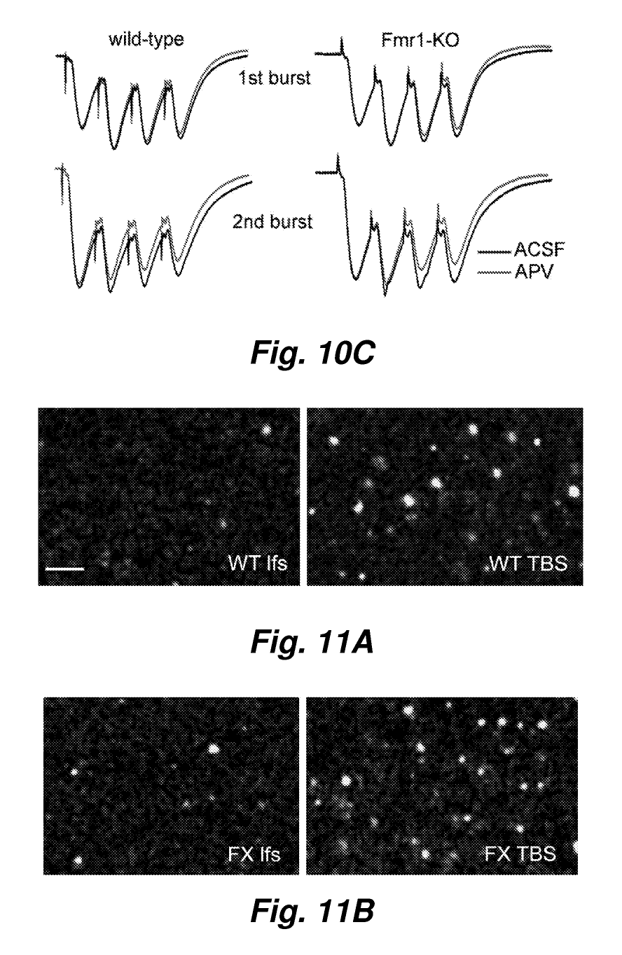

We next attempted to identify which of the steps in LTP production were negatively affected by the mutation. There were no evident between-group differences in the waveforms of the composite postsynaptic responses (four overlapping field EPSPs) generated by theta bursts, as can be seen from the averaged traces in FIG. 10A. The mean sizes of the initial burst responses in the train of five were comparable for WT cases (+64.6±2.5 mV/ms) and mutants (+60.5±5.9 mV/ms), as was the degree to which the burst responses facilitated during the trains. FIG. 10B describes the size (area) of burst responses two through five as a fraction of the size of the first burst in the train.

The mean percent facilitation across burst responses 2-5 was +82.1±9.0% (median, +78) for the WT slices and +70.0±10.7% (median, +82) for the Fmr1-KOs. Estimates of the extent to which TBS engaged NMDA receptors in the mutants were made by comparing burst responses in the presence and absence of the selective antagonist APV. As shown in FIG. 10C, the antagonist caused a marked, and reversible, depression of the response to the second of two theta bursts. This is consistent with earlier work showing that feedforward IPSPs, once having been activated by an initial burst, enter a refractory period that has its peak near the onset time of a second theta burst (Larson and Lynch, 1986). This reduces the GABAergic conductance that normally shunts AMPA receptor-mediated excitation, and thereby reduces the prolonged depolarization needed to unblock NMDA receptors. The results summarized in FIG. 10C indicate that all of these processes are operational in Fmr1-KO mice.

The finding that burst responses were of comparable size, and increased by the same increment, across burst 1 to burst 2 in a theta train indicates that GABAA receptor mediated IPSPs, as well as the after hyperpolarizations that follow cell spiking, are not significantly different between genotypes. To further assess the representation of GABAergic elements between genotypes, tissue sections from WT and Fmr1-KO mice were processed for the immunocytochemical localization of the GABA biosynthetic enzyme GAD using antisera that detects both the 65 and 67 kDa isoforms, and immunolabeled puncta within the proximal stratum radiatum were counted. Quantification of both numbers and labeling densities of GAD-immunoreactive puncta revealed no differences between genotypes for these measures.

Actin polymerization in dendritic spines is an essential step in the stabilization of TBS-induced LTP in rats (Fukazawa et al., 2003; Lin et al., 2005a; Kramár et al., 2006) and mice (Lynch et al., 2007b). Previous studies have shown that theta stimulation activates the p21-activated kinase (PAK)/cofilin pathway, which regulates the growth of actin filaments in dendritic spines (Chen et al., 2007; Rex et al., 2007). Phosphorylation inhibits the activity of cofilin, an actin-depolymerizing factor, and thus promotes actin polymerization. We tested whether this signaling pathway was engaged by TBS in the fragile X mutant. Hippocampal slices from WT and Fmr1-KO mice received five theta bursts and were left in the chamber for 7 min, a time point at which the phosphorylation of cofilin is maximal after TBS in rat (Chen et al., 2007; Rex et al., 2007). Slices were then fixed and processed for the localization of phosphorylated (p-) cofilin using immunofluorescence techniques.

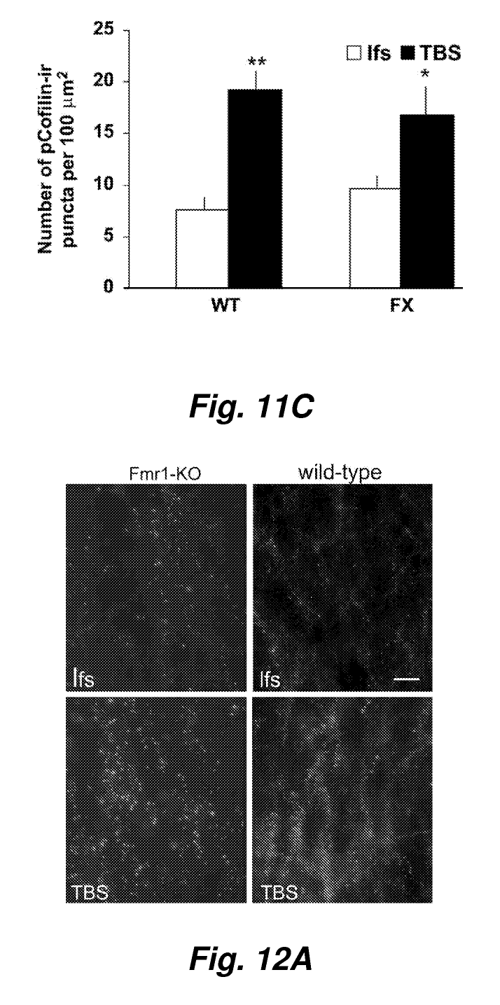

As shown in the photomicrographs of FIGS. 11A and 11B, p-cofilinimmunoreactive puncta were more numerous in stratum radiatum of CA1 in slices receiving TBS than in those receiving low-frequency control stimulation. Quantification indicated that basal numbers were not significantly different between genotypes (p=0.32 for low-frequency stimulation groups, Student's ttest), and that TBS resulted in significant increases in labeled puncta for both WT and Fmr1-KO mice (p=0.0019 for WT control vs TBS groups, Student's t test; p=0.033 for Fmr1-KO control versus TBS groups, Student's ttest). Moreover, the effect of stimulation was similar between genotypes (p=0.323 for WT and Fmr1-KO TBS groups). These data indicate that the PAK/cofilin pathway is not perturbed in the mutant.

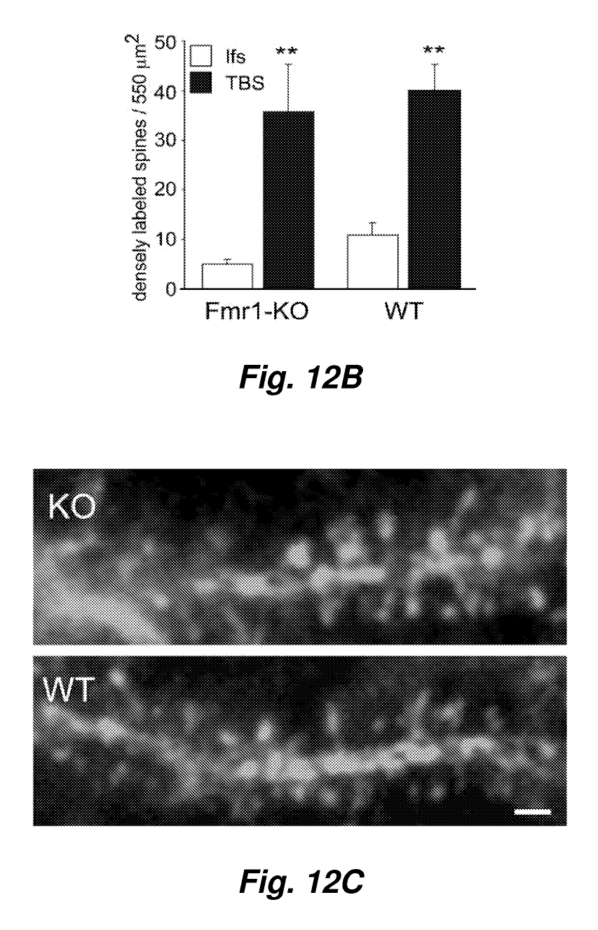

Next, we tested whether a deficit in actin polymerization might account for the rapid decay of potentiation in the Fmr1-KO mice. Alexa-568-labeled phalloidin was applied to slices beginning 30 min after delivery of five theta bursts to each of two converging populations of Schaffer-commissural afferents to the target field in CA1b stratum radiatum. The tissue was then fixed, sectioned, and examined under epifluorescence illumination. FIG. 12A shows representative photomicrographs from number of densely labeled puncta localized in the proximal stratum radiatum, the dendritic zone containing the stimulated synapses. Close examination indicated that the labeled structures had the size and appearance of dendritic spines. Quantification of intensely phalloidin-labeled spines demonstrated that both genotypes expressed low basal numbers in control slices receiving baseline low-frequency stimulation (FIG. 12B) (11±2 vs 5±1, mean ±SEM, per 550 m2 for WT vs Fmr1-KO). TBS-treated slices from fragile X mutants showed dramatically elevated numbers of labeled spines (36±10/5 50 m2) versus unstimulated slices [p<0.01, Tukey's honest significant difference (HSD)]; the theta induced increases in labeled spines were not statistically different from values obtained for the TBS-treated WT slices (40±5; p>0.3, ANOVA). Mean values obtained for WT and Fmr1-KO slices receiving 10 theta bursts showed no additional elevations from the five burst cases (data not shown), consistent with previous findings in rats (Kramar et al., 2006). Finally, although spine morphology was not formally assessed in this material, a range of spine types and sizes was observed in both genotypes as shown in FIG. 12C. Overall, these results suggest that TBS-induced actin polymerization, which normally accompanies synaptic potentiation, remains functional in the Fmr1-Kos, despite impairments in LTP.

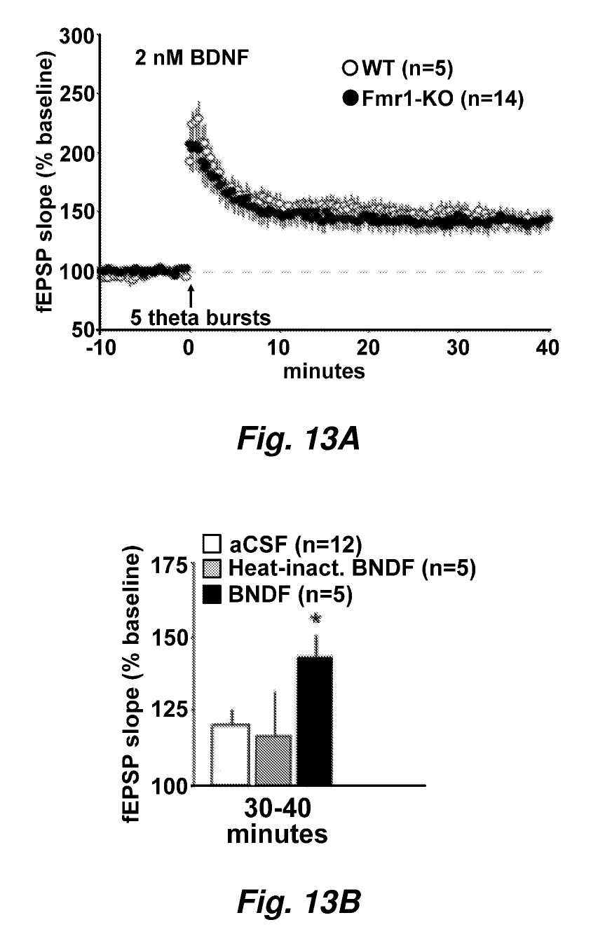

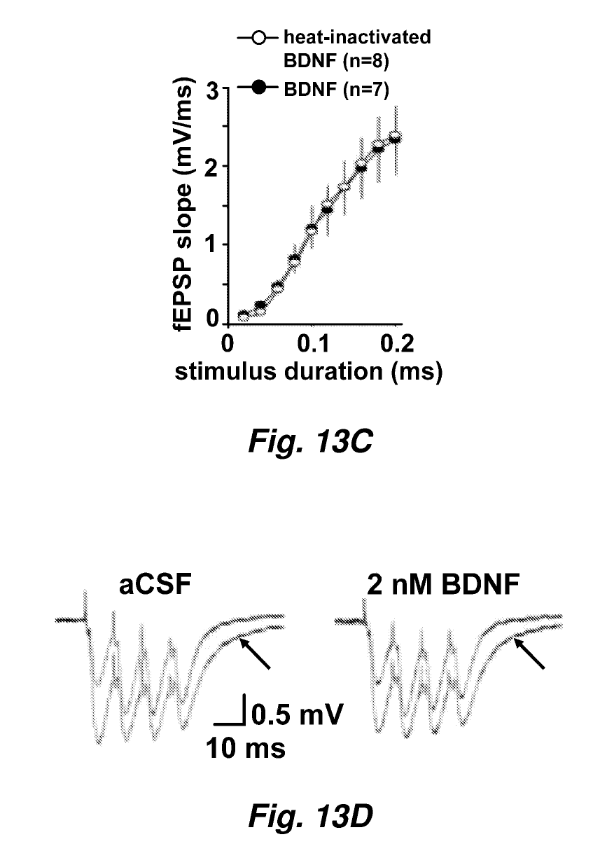

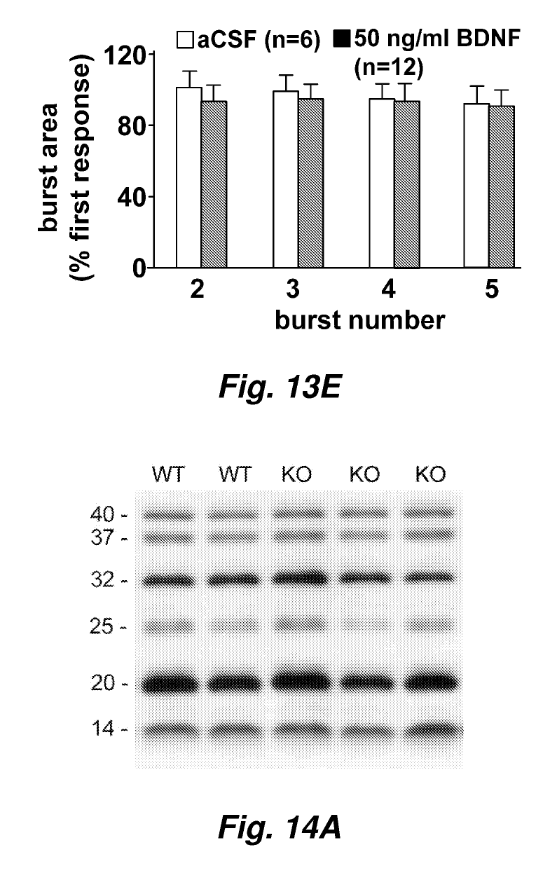

The above results indicate that the complex machinery that induces, expresses, and begins the process of consolidating LTP is intact in the fragile X mutant mouse and is set in motion by five bursts of theta stimulation. However, it appears that some step in addition to or occurring beyond polymerization does not receive sufficient drive, in the five burst case, for activation, resulting in a potentiation that gradually decays. This raises the experimental question of whether increased levels of positive modulation can be used to enhance the effects of the near-threshold level of theta stimulation. We explored this possibility using BDNF, a neurotrophin that is released by theta burst stimulation (Balkowiec and Katz, 2002; Aicardi et al., 2004), potently promotes LTP in rat brain slices (Figurov et al., 1996; Chen et al., 1999; Kramár et al., 2004) and regulates plasticity-associated spine actin polymerization (Rex et al., 2007). FIG. 13 summarizes results from experiments in which BDNF (50 ng/ml) was bath-applied to WT or Fmr1-KO slices continuously through a recirculating perfusion system. In WT slices treated with BDNF, the delivery of five theta bursts produced potentiation (+51.3±8.6% for minutes 30-40 postTBS) (FIG. 13A) that was similar to that elicited by five theta bursts in the absence of exogenous BDNF (p>0.5 vs ACSF at 30-40 min; repeated-measures ANOVA) (compare FIG. 9C). This result accords with other reports (Pang et al., 2004; Lynch et al., 2007b) that, in marked contrast to results obtained with rat slices, the magnitude of LTP in wild-type mice is not elevated by low concentrations of BDNF. Fmr1-KO slices bathed in BDNF exhibited a degree of LTP (+42.1±8.6% for minutes 30-40) (FIG. 13A, B) that was equivalent to that in the WT slices, and was substantially greater than potentiation in mutant slices infused with either ACSF alone (p=0.009 for minutes 30-40 post-TBS) or with ACSF containing heat-inactivated BDNF (p<0.05) (FIG. 13B). BDNF did not measurably affect facilitation of burst responses during the theta trains in either WT (p>0.4) or Fmr1-KO mice (p>0.4 vs ACSF and heat-inactivated BDNF, two-way repeated measures ANOVA); FIGS. 13D and 13E, shows comparison of ACSF and BDNF effects in mutant slices for these measures. This again stands in marked contrast to results obtained with rat slices. Finally, BDNF did not alter input-output curves compared with slices infused with ACSF alone (p>0.3 for both WT and Fmr1-KO, respectively) (data not shown) or with 50 ng/ml heat-inactivated BDNF (p>0.3 for Fmr1-KO) (FIG. 13C). It thus appears that BDNF selectively corrected the impairment to LTP consolidation in the mutants.

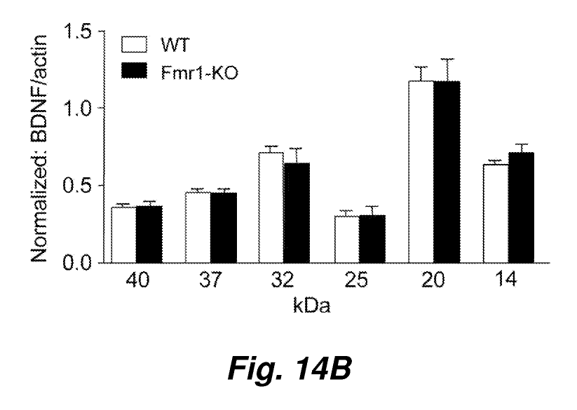

The restorative effects of BDNF in Fmr1-KO slices suggest the possibility that the synaptic deficits seen in these mice arise from impaired production of the neurotrophin. Accordingly, we compared levels of precursor and mature BDNF (14 kDa form) in hippocampus of Fmr1-KOs and WTs with Western blots (FIG. 14A). In homogenates from WT and Fmr1-KO mice, there were several precursor forms of BDNF immunoreactivity ranging from 40 to 20 kDa, with two major bands at 32 and 20 kDa. Whereas non-neuronal cells transfected to over express proBDNF (Mowla et al., 2001) generate a major band of immunore activity at 32 kDa, other studies have identified BDNF precursors in the range of 30-38 kDa, and smaller proteolytic fragments ranging from 17 to 28 kDa (Biagini et al., 2001; Mowla et al., 2001; Pang et al., 2004; Zhou et al., 2004b; Pollak et al., 2005; Teng et al., 2005). Therefore, to include the various pro-BDNF forms present in situ, we analyzed all bands from 20 to 40 kDa in addition to mature BDNF. Levels of immunoreactivity were normalized to actin that served as a loading control; parallel analyses demonstrated that whole homogenate actin levels were not significantly different between genotypes (p=0.8). As shown in FIG. 14B, concentrations of pro-BDNF and mature BDNF immunoreactivity were not statistically different between genotypes (p>0.05 for WT versus KO, for all bands evaluated). These data indicate that expression and post-translational processing of BDNF are not disturbed in the hippocampus of the fragile X mutant mouse. Finally, total levels of BDNF's TrkB receptor were assessed by Western blotting in the same samples analyzed for BDNF content. Quantification of both full-length and truncated isoforms (145 and 95 kDa, respectively) identified no difference in TrkB levels between genotypes.

### Discussion

Several factors and experimental conditions have been identified that modulate, but are not obligatory for, the induction and/or stabilization of LTP. BDNF, for example, is necessary for production of LTP by the tabursts, but is not required when long trains of high-frequency stimulation are used (Chen et al., 1999). Similarly, deficits in LTP in the aged hippocampus that are seen with modest stimulation protocols can be overcome by more intense afferent stimulation (Tombaugh et al., 2002). The defect related to the fragile X mutation also appears to involve a factor that contributes to, but is not essential for, the development of stable potentiation. Conventional 10 burst theta trains produced a normal-appearing LTP in mutant slices that, although elicited by a threshold number of bursts (Arai and Lynch, 1992), was markedly impaired. Notably, at the threshold, five burst trains more closely approximate conditions likely to occur in vivo than do full-length trains. Chronic recording studies have shown that a pattern similar to theta bursting occurs in hippocampus during learning, and that this typically involves small numbers of serial bursts (e.g., individual cells tend to fire in series of three to four bursts at theta frequency) (Otto et al., 1991). Such short trains are at threshold for producing LTP, although they can, with repetition over several minutes, incrementally produce full strength potentiation (Larson et al., 1986; Larson and Lynch, 1989; Colgin et al., 2003). In all, then, the fragile X impairment described here can reasonably be assumed to emerge as a plasticity deficit during learning and, thus, could be an important component in the behavioral aspects of the disease.

Possibly related to these observations, Meredith et al. (2007) reported previously that deficits in spike timing potentiation in the frontal cortex of Fmr1-KOs are only evident with threshold levels of stimulation and can be overcome with stronger stimulation.

Several LTP-related physiological variables known to be sensitive to experimental manipulation were not affected by the mutation. The theta burst response, which is shaped by a complex set of presynaptic and postsynaptic variables (Arai and Lynch, 1992; Lynch et al., 2007b), was by appearance and measurement not different between WTs and Fmr1-KOs. The facilitation of these composite responses over the course of a theta train, an event that involves suppression of inhibitory transmission via activation of GABA autoreceptors (Larson et al., 1986; Mott and Lewis, 1991), was similarly normal in the mutants. Moreover, the NMDA receptor-mediated component of the burst response (Larson and Lynch, 1998) and its facilitation during the theta train was present in the mutants and not evidently different in size or waveform from what is found in WT slices. These results point to a process other than initial induction as the element in LTP production that is affected by the fragile X mutation.

FMRP binds to mRNA as part of a ribonucleoprotein complex, but little is known regarding the proteins affected by its activity (Zalfa et al., 2003, 2007). There is, however, evidence that actin dynamics are sensitive to the fragile X mutation. Changes in the actin cytoskeleton produced by an extracellular signal, and mediated by the small GTPase Rac, are distorted in murine fibroblasts from fragile X mutants. Levels of phosphorylated (inactivated) cofilin, a protein that plays a key role in regulating the assembly of actin filaments, are abnormally low in these preparations, whereas concentrations of cofilin phosphatase are elevated (Castets et al., 2005). It is also the case that FMRP in Drosophila negatively regulates expression of the profilin homolog, a protein critical to the elongation of actin filaments (Reeve et al., 2005). The above proteins, along with actin itself, are enriched in dendritic spines (Racz and Weinberg, 2006; Chen et al., 2007), and perturbations to their activities could explain the abnormal spine morphologies associated with FXS. Disturbances to actin dynamics are also relevant to the observed deficits in LTP. Results from a series of electron microscopic studies indicate that rapid changes in the morphology of dendritic spines and their postsynaptic densities occur in conjunction with LTP (Lee et al., 1980; Desmond and Levy, 1986; Toni et al., 2001; Park et al., 2006); experiments with new light-microscopic techniques have confirmed changes in spine shape (Zhou et al., 2004a; Ehrlich et al., 2007) and increases in the size of the synapse (Chen et al., 2007). Other work indicates that theta bursts cause a rapid (<2 min) polymerization of actin in spine heads (Lin et al., 2005a), something that is a very likely prerequisite to shape change. The threshold for this effect is the same as that for induction of LTP and treatments that block theta-induced polymerization, even if applied after the stimulation bursts, reverse LTP (Kramar et al., 2006). Given these points, a reasonable explanation for the loss of LTP (at threshold stimulation levels) would be that the FXS mutation depressed signaling pathways needed to modify spine morphology. However, even modest theta trains produced robust and normal-appearing increases in spine p-cofilin and F-actin levels in the mutant slices. Together with the results from the analysis of burst responses, these findings indicate that the LTP machinery, in Fmr1-KOs, is intact from the relatively brief physiological events required for induction through the complex signaling cascades needed for actin polymerization.

Given the above conclusion, it seems reasonable to look beyond actin assembly for the cellular defect that impairs LTP in fragile X mice. Work showing that theta-induced polymerization can be reversed in the first few minutes after its occurrence (Kramár et al., 2006) suggests that cross-linking, capping, and other activities that stabilize the cytoskeleton play a major role in LTP consolidation. Proteins involved in actin cross-linking, including spectrin (Siman et al., 1987; Walsh and Kuruc, 1992), adducin (Wyneken et al., 2001), actinin (Wyszynski et al., 1998), dystrophin (Jancsik and Hajos, 1998) and others, are concentrated in spines and/or postsynaptic densities. Of these proteins, at least one has been identified as potentially being regulated by FMRP: Antibody-positioned RNA amplification indicates that spectrin(a-fodrin) mRNA is among the RNA cargo of FMRP Miyashiro et al., 2003). Moreover, although the LTP deficit in the mutants emerged before time points typically considered dependent on protein synthesis, there is evidence that under some stimulation conditions, local translation contributes to early processes of LTP stabilization (Woo and Nguyen, 2003; Kelleher et al., 2004). Together, these results raise the possibility that the FMRP mutation disrupts availability of locally translated actin cross-linking proteins and, consequently, cytoskeletal stabilization. Alternatively, the neuronal actin cytoskeleton is sensitive to calcium (Rosenmund and Westbrook, 1993; Furukawa et al., 1995) and there is indirect evidence that regulation of the cation is disturbed in cortex of fragile X mice (Meredith et al., 2007). Whatever their origins, the cytoskeletal problems found in the mutants appear to be partial because longer trains of afferent stimulation can overcome them to produce stable potentiation.

The evidence that the hippocampal LTP deficits in fragile X are both discrete and partial encourages the idea that they might be offset with one or more physiologically plausible treatments. BDNF acts via Rho GTPases to regulate the assembly of the actin cytoskeleton in developing neurons (Ozdinler and Erzurumlu, 2001; Gehler et al., 2004; Miyamoto et al., 2006), and there is previous evidence that aspects of these signaling pathways are retained into adulthood in hippocampus (Rex et al., 2007). These observations point to BDNF as a logical candidate for atreatment to offset the problems in spine reorganization hypothesized to arise from the fragile X mutation. The neurotrophin positively modulates the formation of LTP in normal rodents (Korte et al., 1995; Patterson et al., 1996; Kang et al., 1997), and is found to offset deficits in LTP in murine models of Huntington's disease (Lynch et al., 2007b), possibly via effects on the actin cytoskeleton (Rex et al., 2007). Consistent with these points, we found that brief infusions of 50 ng/ml BDNF fully restored LTP in Fmr1-KOs and did so without causing evident changes to baseline physiology or theta burst responses.

The latter results raise the question of whether it will be possible to treat the plasticity deficits in FXS by upregulating expression of BDNF. An approach of this type, using positive modulators of AMPA-type glutamate receptors to stimulate neurotrophin production, was reported previously to reverse age-related impairments to LTP in rat (Rex et al., 2006). Because BDNF protein has a relatively long half-life (Nawa et al., 1995; Sano et al., 1996), it was possible in those studies to stably increase neurotrophin levels using twice a day treatments with a short half-life compound (Rex et al., 2006). Given that the loss of FMRP does not appear to affect mature BDNF protein levels or processing, or levels of its high-affinity receptor TrkB, efforts to increase its production have a reasonable chance of being successful. This point supports the idea of using activity modulation and an endogenous BDNF-based strategy for the treatment of mental retardation in fragile X syndrome.

### REFERENCES

Aicardi G, Argilli E, Cappello S, Santi S, Riccio M, Thoenen H, Canossa M (2004) Induction of long-term potentiation and depression is reflected by corresponding changes in secretion of endogenous brain-derived neurotrophic factor. Proc Natl Acad Sci USA 101:15788-15792.
Arai A, Lynch G (1992) Factors regulating the magnitude of long-term potentiation induced by theta pattern stimulation. Brain Res 598:173-184.
Balkowiec A, Katz D (2002) Cellular mechanisms regulating activity-dependent release of native brain-derived neurotrophic factor from hippocampal neurons. J Neurosci 22:10399-10407.
Biagini G, Avoli M, Marcinkiewicz J, Marcinkiewicz M (2001) Brain-derived neurotrophic factor superinduction parallels anti-epileptic-neuroprotective treatment in the piocarpine epilepsy model. J Neurochem 76: 1814-1822.
Bramham C, Messaoudi E (2005) BDNF function in adult synaptic plasticity: the synaptic consolidation hypothesis. Prog Neurobiol 76:99-125. Carlisle H, Kennedy M (2005) Spine architecture and synaptic plasticity. Trends Neurosci 28:182-187.
Castets M, Schaeffer C, Bechara E, Schenck A, Khandjian E, Luche S, Moine H, Rabilloud T, Mandel J, Bardoni B (2005) FMRP interferes with the RacI pathway and controls actin cytoskeleton dynamics in murine fibroblasts. Hum Mol Genet. 14:835-844.
Chen G, Kolbeck R, Barde Y A, Bonhoeffer T, Kossel A (1999) Relative contribution of endogenous neurotrophins in hippocampal long-term potentiation. J Neurosci 19:7983-7990.
Chen L Y, Rex C S, Casale M, Gall C M, Lynch G (2007) Changes in synaptic morphology accompany actin aignaling during LTP. J Neurosci 27:5363-5372.
Colgin L, Kubota D, Lynch G (2003) Cholinergicplasticityinthehippocampus. Proc Natl Acad Sci USA 100:2872-2877.
Comery T, Harris J, Willems P, Oostra B, Irwin S, Weiler I, Greenough W (1997) Abnormal dendritic spines in fragile X knock-out mice: maturation and pruning deficits. Proc Natl Acad Sci USA 94:5401-5404.
Cooke S, Bliss T (2006) Plasticity in the human central nervous system. Brain 129:1659-1673.
Desai N, Casimiro T, Gruber S, Vanderklish P (2006) Early postnatal plasticityinneocortex of Fmr1 knockoutmice. J Neurophysiol 96:1734-1745.
Desmond N, Levy W (1986) Changes in the postsynaptic density with long-term potentiation in the dentate gyrus. J Comp Neurol 253:476-482.
Ehrlich I, Klein M, Rumpel S, Malinow R (2007) PSD-95 is required for activity-driven synapse stabilization. Proc Natl Acad Sci USA 104:4176-4178.

FigurovA, Pozzo-Miller L D, Olafsson P, WangT, Lu B (1996) Regulation of synaptic responses to high-frequency stimulation and LTP by neurotrophins in the hippocampus. Nature 381:706-709.
Fukazawa Y, Saitoh Y, Ozawa F, Ohta Y, Mizuno K, Inokuchi K (2003) Hippocampal LTP is accompanied by enhanced F-actin content within the dendritic spine that is essential for late LTP maintenance in vivo. Neuron 38:447-460.
Furukawa K, Smith-Swintosky V L, Mattson M P (1995) Evidence that actin depolymerization protects hippocampal neurons against excitotoxicity by stabilizing [Ca²˜]i. Exp Neurol 133:153-163.
Gehler S, Shaw A, Sarmiere P, Bamburg J, Letourneau P (2004) Brain-derived neurotrophic factor regulation of retinal growth cone filopodial dynamics is mediated through actin depolymerizingfactor/cofilin. J Neurosci 24:10741-10749.
Godfraind J, Reyniers E, De Boulle K, D'Hooge R, De Deyn P, Bakker C, Oostra B, Kooy R, Willems P (1996) Long-term potentiation in the hippocampus of fragile X knockout mice. Am J Med Genet. 64:246-251.
Grossman A, Elisseou N, McKinney B, Greenough W (2006) Hippocampal pyramidal cells in adult Fmr1 knockout mice exhibit an immature-appearing profile of dendritic spines. Brain Res 1084:158-164.
Hinton V, Brown W, Wisniewski K, Rudelli R (1991) Analysis of neocortex in three males with fragile X syndrome. Am J Med Genet. 41:289-294.
Irwin S, Patel B, Idupulapati M, Harris J, Crisostomo R, Larsen B, Kooy F, Willems P, Cras P, Kozlowski P, Swain R, Weiler I, Greenough W (2001) Abnormal dendritic spine characteristics in the temporal and visual cortices of patients with fragile-X syndrome: a quantitative examination. Am J Med Genet. 98:161-167.
Irwin S, Idupulapati M, Gilbert M, Harris J, Chakravarti A, Rogers E, Crisostomo R, Larsen B, Mehta A, Alacantara C, Patel B, Swain R, Weiler I, Oostra B, GreenoughW (2002) Dendritic spine and dendritic field characteristics on layer V pyramidal neurons in the visual cortex of fragile-X knockout mice. Am J Med Genet. 111: 140-146.
Jain A K (1984) Fundamentals of digital image processing. Englewood Cliffs, N.J.: Prentice Hall.
JancsikV, Hajos F (1998) Differential distribution of dystrophin in postsynaptic densities of spine synapses. NeuroReport 9:2249-2251.
Kang H J, Welcher A A, Shelton D, Schuman E M (1997) Neurotrophins and time: different roles for TrkB signaling in hippocampal long-term potentiation. Neuron 19:653-664.
Kelleher III R J, Govindarajan A, Jung H Y, Kang H, Tonegawa S (2004) Translational control by MAPK signaling in long-term synaptic plasticity and memory. Cell 116:467-479.
Korte M, Carroll P, Wolf E, Brem G, Thoenen H, Bonhoeffer T (1995) Hippocampal long-term potentiation is impaired in mice lacking brain-derived neurotrophic factor. Proc Natl Acad Sci USA 92:8856-8860.
Kramár E, Lynch G (2003) Developmental and regional differences in the consolidation of long-term potentiation. Neuroscience 118:387-398.
Kramár E, Lin B, Lin C, Arai A, Gall C, Lynch G (2004) A novel mechanism for the facilitation of theta-induced long-term potentiation by brain-derived neurotrophic factor. J Neurosci 24:5151-5161.
Kramár E, Lin B, Rex C, Gall C, Lynch G (2006) Integrin-driven actin polymerization consolidates long-term potentiation. Proc Natl Acad Sci USA 103:5579-5584.
Larson J, Lynch G (1986) Induction of synaptic potentiation in hippocampus by patterned stimulation involves two events. Science 232:985-988.
Larson J, Lynch G (1989) Theta pattern stimulation and the induction of LTP: the sequence in which synapses are stimulated determines the degree to which they potentiate. Brain Res 489:49-58.
Larson J, Lynch G (1998) Role of N-methyl-D-aspartate receptors in the induction of synaptic potentiation by burst stimulation patterned after the hippocampal theta-rhythm. Brain Res 441:111-118.
Larson J, Wong D, Lynch G (1986) Patterned stimulation at the theta frequency is optimal for the induction of hippocampal long-term potentiation. Brain Res 368:347-350.
Larson J, Jessen R, Kim D, Fine A, du Hoffmann J (2005) Age-dependent and selective impairment of long-term potentiation in the anterior piriform cortex of mice lacking the fragile X mental retardation protein. J Neurosci 25:9460-9469.
Lee K, Schottler F, Oliver M, Lynch G (1980) Brief bursts of high-frequency stimulation produce two types of structural changes in rat hippocampus. J Neurophysiol 44:247-258.
Li J, Pelletier M R, Perez Velazquez J L, Carlen P L (2002) Reduced cortical synaptic plasticity and GluR1 expression associated with fragile X mental retardation protein deficiency. Mol Cell Neurosci 19:138-151.
Lin B, Kramár E, BiX, Brucher F, Gall C, Lynch G (2005a) Theta stimulation polymerizes actin in dendritic spines of hippocampus. J Neurosci 25:2062-2069.
Lin C Y, Lynch G, Gall C (2005b) AMPA receptor stimulation increases α5β1 integrin surface expression, adhesive function and signaling. J Neurochem 94:531-546.
Lund R (1978) Development and plasticity of the brain. New York: Oxford UP.
Lynch G, Rex C, Gall C (2007a) LTP consolidation: substrates, explanatory power, and functional significance. Neuropharmacology 52:12-23.
Lynch G, Kramar E A, Rex C S, Jia Y, Chappas D, Gall C M, Simmons D A (2007b) Brain-derived neurotrophic factor restores synaptic plasticity in a knock-in mouse model of Huntington's disease. J Neurosci 27:4424-4434.
Marin-Padilla M (1972) Structural abnormalities of the cerebral cortex in human chromosomal aberrations. Brain Res 44:625-629.
Marin-Padilla M (1974) Structural organization of the cerebral cortex (motor area) in human chromosomal aberrations. A Golgi study. Brain Res 66:375-391.
Meng Y, Zhang Y, Tregoubov V, Falls D, Jia Z (2003) Regulation of spine morphology and synaptic function by LIMK and the actin cytoskeleton. Rev Neurosci 14:233-240.
Meredith R M, Holmgren C D, Weidum M, Burnashev N, Mansvelder H D (2007) Increased threshold for spike-timing-dependent plasticity is caused by unreliable calcium signaling in mice lacking fragile X gene FMR1. Neuron 54:627-638.
Michalski B, FahnestockM (2003) Pro-brain-derivedneurotrophicfactor is decreased in parietal cortex in Alzheimer's disease. Mol Brain Res 111:148-154.
Miyamoto Y, Yamauchi J, Tanoue A, Wu C, Mobley W (2006) TrkB binds and tyrosine-phosphorylates Tiam1, leading to activation of Rac1 and induction of changes in cellular morphology. Proc Natl Acad Sci USA 103:10444-10449.
Miyashiro K, Beckel-Mitchener A, Purk T, Becker K, Barret T, Liu L, Carbonetto S, Weiler I, Greenough W, Eberwine J (2003) RNA cargoes associating with FMRP reveal deficits in cellular functioning in Fmr1 null mice. Neuron 37:417-431.
Mott D, Lewis D (1991) Facilitation of the induction of long-term potentiation by GABAB receptors. Science 252:1718-1720.
Mowla S, Farhadi H, Pareek S, Atwal J, Morris S, Seidah N, MurphyR (2001) Biosynthesis and post-translational processing of the precursor to brain-derived neurotrophic factor. J Biol Chem 276:12660-12666.
Muddashetty R, Kelie S, Gross C, Xu M, Bassell G (2007) Dysregulated metabotropic glutamate receptor-dependent translation of AMPA receptor and postsynaptic density-95 mRNAs at synapses in a mouse model of fragile X syndrome. J Neurosci 27:5338-5348.
Nawa H, Carnahan J, Gall C (1995) BDNF protein measured by a novel enzyme immunoassay in normal brain and after seizure: partial disagreement with mRNA levels. Eur J Neurosci 7:1527-1535.
Oostra B, Hoogeveen A (1997) Animal model for fragile X syndrome. Ann Med 29:563-567.
Otto T, Eichenbaum H, Wiener S I, Wible C G (1991) Learning-related patterns of CA1 spike trains parallel stimulation parameters optimal for inducing hippocampal long-term potentiation. Hippocampus 1: 181-192.
Ozdinler P, Erzurumlu R (2001) Regulation of neurotrophin-induced axonal responses via Rho GTPases. J Comp Neurol 438:377-387.
Pang P, Teng H, Zaitsev E, Woo N, Sakata K, Zhen S, Teng K, Yung W, Hempstead B, Lu B (2004) Cleavage of proBDNF by tPA/plasmin is essential for long-term hippocampal plasticity. Science 306:487-491.
Paradee W, Melikian H E, Rasmussen D L, Kenneson A, Conn P J, Warren S T (1999) Fragile X mouse: strain effects of knockout phenotype and evidence suggesting deficient amygdala function. Neuroscience 94:185-192.
Park M, Salgado J M, Ostroff L, Helton T D, Robinson C G, Harris K M, Ehlers M D (2006) Plasticity-induced growth of dendritic spines by exocytic trafficking from recycling endosomes. Neuron 52:817-830.
Patterson S L, Abel T, Deuel T A, Martin K C, Rose J C, Kandel E R (1996) Recombinant BDNF rescues deficits in basal synaptic transmission and hippocampal LTP in BDNF knockout mice. Neuron 16:1137-1145.
Pollak D, Herkner K, Hoeger H, Lubec G (2005) Behavioral testing upregulates pCaMKII, BDNF, PSD-95 and egr-1 in hippocampus of FVB/N mice. Behav Brain Res 163:128-135.
Racz B, Weinberg R (2006) Spatial organization of cofilin in dendritic spines. Neuroscience 138:447-456.
Reeve S, Bassetto L, Genova G, Kleyner Y, Leyssen M, Jackson F, Hassan B (2005) The_{Drosophüa}fragileXmental retardation protein controls actin dynamics by directly regulating profilin in the brain. Curr Biol 15:1156-1163.
Rex C, Kramar E, Colgin L, Lin B, Gall C, Lynch G (2005) Long-term potentiation is impaired in middle-aged rats: regional specificity and reversal by adenosine receptor antagonists. J Neurosci 25:5956-5966.
Rex C S, Lauterbom J C, Lin C Y, Kramar E A, Rogers G A, Gall C M, Lynch G (2006) Restoration of long-term potentiation in middle-aged hippocampus after induction of brain-derived neurotrophic factor. J Neurophysiol 96:677-685.
Rex C S, Lin C Y, Kramar E A, Chen L Y, Gall C M, Lynch G (2007) Brain-derived neurotrophic factor promotes long-term potentiation-related cytoskeletal changes in adult hippocampus. J Neurosci 27:30 17-3029.
Reymann K G, Frey J U (2007) The late maintenance of hippocampal LTP: requirements, phases, “synaptic tagging,” “late-associativity” and implications. Neuropharmacology 52:24-40.
Rosenmund C, Westbrook G (1993) Calcium-induced actin depolymerization reduces NMDA channel activity. Neuron 10:805-814.
Rudelli R, Brown W, Wisniewski K, Jenkins E, Laure-Kamionowska M, Connell F, Wisniewski H (1985) Adult fragile X syndrome. Cliniconeuropathologic findings. Acta Neuropathologica 67:289-295.
Sano K, Nanba H, Tabuchi A, Tsuhiya T, TsudaM (1996) BDNF gene can be activated by CA² signals without involvement of de novo AP-1 synthesis. Biochem Biophys Res Comm 229:788-793.
Siman R, Ahdoot M, Lynch G (1987) Ontogeny, compartmentation, and turnover of spectrin isoforms in rat central neurons. J Neurosci 7:55-64.
Staubli U, Perez Y, Xu F, Rogers G, Ingvar M, Stone-Elander S, Lynch G (1994) Centrally active modulators of glutamate (AMPA) receptors facilitate the induction of LTP in vivo. Proc Natl Acad Sci USA 91:11158-11162.
Teng H, Teng K, Lee R, Wright S, Tevar S, Almeida R, Kermani P, Torkin R, ChenZ-Y, Lee F, Kraemer R, Nykjaer A, Hempstead B (2005) ProBDNF induces neuronal apoptosis via activation of a receptor complex of p75NTR and sortilin. J Neurosci 25:5455-5463.
The Dutch-Belgian Fragile X Consortium (1994) FMR1 knockout mice: a model to study fragile X mental retardation. Cell 78:23-33.
Todd P, Malter J (2002) Fragile X mental retardation protein in plasticity and disease. J Neurosci Res 70:623-630.
Tombaugh G, Rowe W, Chow A, Michael T, Rose G (2002) Theta-frequency synaptic potentiation in CA1 in vitro distinguishes cognitively impaired from unimpaired aged Fisher 344 rats. J Neurosci 22:9932-9940.
Toni N, Buchs P, Nikonenko I, Povilaitite P, Parisi L, Muller D (2001) Remodeling of synaptic membranes after induction of long-term potentiation. J Neurosci 21:6245-6251.
Walsh M, Kuruc N (1992) The postsynaptic density: constituent and associated proteins characterized by electrophoresis, immunoblotting, and peptide sequencing. J Neurochem 59:667-678.
Weiler I, Spangler C, Klintsova A, Grossman A, Kim S, Bertaina-Anglade V, Khaliq H, de Vries F, Lambers F, Hatia F, Base C, Greenough W (2004) Fragile X mental retardation protein is necessary for neurotransmitter-activated protein translation at synapses. Proc Natl Acad Sci USA 101: 17504-17509.
Woo N H, Nguyen P V (2003) Protein synthesis is required for synaptic immunityto depotentiation. J Neurosci 23:1125-1132.
Wyneken U, Smalla K, Marengo J, Soto D, de la Cerda A, Tischmeyer W, Grimm R, Boeckers T, Wolf G, Orrego F, Gundelfinger E (2001) Kainate-induced seizures alter protein composition and N-methyl-Daspartate receptor function of rat forebrain postsynaptic densities. Neuroscience 102:65-74.
Wyszynski M, Kharazia V, Shanghvi R, Rao A, Beggs A, Craig A, Weinberg R, Sheng M (1998) Differential regional expression and ultrastructural localization of alpha-actinin-2, a putative NMDA receptor-anchoring protein, in rat brain. J Neurosci 18:1383-1392.
Zalfa F, Giorgi M, Primerano B, Moro A, Di Penta A, Reis S, Oostra B, Bagni C (2003) The fragile X syndrome protein FMRP associates with BC1 RNA and regulates the translation of specific mRNAs at synapses. Cell 112:317-327.
Zalfa F, Eleuteri B, Dickson K, Mercaldo V, De Rubeis S, di PentaA, Tabolacci E, Chiurazzi P, Neri G, GrantS, Bagni C (2007) A new function for the fragile X mental retardation protein in regulation of PSD-95 mRNA stability. Nat Neurosci 10:578-587.
Zhao M, Toyoda H, Ko S, Ding H, Wu L, Zhuo M (2005) Deficits in trace fear memory and long-term potentiation in a mouse model for fragile X syndrome. J Neurosci 25:7385-7392.
Zhou Q, HommaK, Poo M (2004a) Shrinkage of dendritic spines associated with long-term depression of hippocampal synapses. Neuron 44:749-757.
Zhou X, SongX, Zhong J, Barati S, Zhou F, Johnson S (2004b) Distribution and localization of pro-brain-derived neurotrophic factor-like immunoreactivity in the peripheral and central nervous system of the adult rat. J Neurochem 91:704-715

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of preserving, improving, or restoring cognitive function in mammal having cognitive impairment and/or a learning disability, said method comprising increasing the level or activity of brain derived neurotrophic factor (BDNF) in the brain of said mammal.

2. The method of claim 5, wherein said mammal shows no substantial neural degeneration.

3. The method of claim 5, wherein said mammal shows essentially no measurable neural degeneration.

4. The method of claim 5, wherein said mammal has a condition selected from the group consisting of Down's syndrome, autism, Rett's syndrome, nonsyndromic X-linked mental retardation, and fragile X syndrome.

5. The method of claim 1, wherein said mammal is a mammal having one or more mutations in the FMR1 gene.

6. The method of claim 5, wherein said mammal is a mammal diagnosed as having, or at risk for, fragile X syndrome.

7. The method of claim 5, wherein said preserving improving, or restoring cognitive function comprises improving long term potentiation in the hippocampus of said mammal.

8. The method of claim 5, wherein said mammal is a mammal not diagnosed and/or under treatment for depression.

9. The method of claim 5, wherein said mammal is a mammal is not diagnosed with an affective disorder.

10. The method of claim 5, wherein said mutation comprises a trinucleotide repeat expansion.

11. The method of claim 5, wherein said mutation is associated with abnormal methylation of said gene.

12. The method of claim 5, wherein said increasing the BDNF level or activity comprises administering one or more glutamate AMPA receptor modulators (ampakines) to said mammal in an amount sufficient to upregulate expression or activity of BDNF in said mammal.

13. The method of claim 12, wherein said one or more glutamate AMPA receptor modulators comprises a high-impact ampakine.

14. The method of claim 12, wherein said one or more glutamate AMPA receptor modulators comprises a high impact ampakine selected from the group consisting of CX516, CX717, and CX691.

15. The method of claim 12, wherein said one or more glutamate AMPA receptor modulators are compounds having the structure IVa or IVb, below: 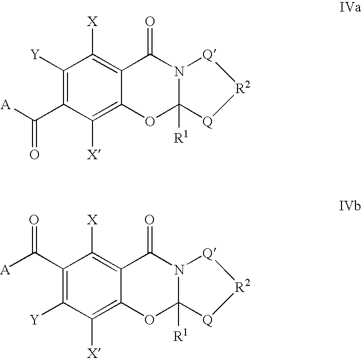
in which:
Q and Q′ are independently hydrogen, —CH₂—, —O—, —S—, alkyl, or substituted alkyl,
R¹ is hydrogen, alkyl or together with Q may be a cycloalkyl ring,
R² may be absent, or if present may be —CH₂—, —CO—, —CH₂CH₂—, —CH₂CO—, —CH₂O—, —CRR′—, or —CONR—,
Y is hydrogen or —OR³, or serves to link the aromatic ring to A as a single bond, ═N— or —NR—,
R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring,
A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur,
R is hydrogen, aryl, arylalkyl, substituted aryl, substituted arylalkyl, alkyl, substituted alkyl, cycloalkyl, substituted cycloalkyl, or heterocycloalkyl,
R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur,
X and X′ are independently R, halo, —CO₂R, —CN, —NRR′, —NRCOR′, —NO₂, —N₃ or —OR.

16. The method of claim 15 with the structure IVa above wherein: Q and Q′ are independently hydrogen, —CH₂—, —O—, —S—, alkyl, or substituted alkyl, R¹ is hydrogen, alkyl or together with Q may be a cycloalkyl ring, R may be absent, or if present may be—CH₂—, —CO_, CH₂CH₂—, —CH₂CO—, —CH₂O—, or —CONR—, Y is hydrogen or —OR³, or serves to link the aromatic ring to A as a single bond, ═N— or —NR—, R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring, A is—NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur, R is hydrogen, aryl, arylalkyl, substituted aryl, substituted arylalkyl, alkyl, substituted alkyl, cycloalkyl, substituted cycloalkyl, or heterocycloalkyl, R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y and which may optionally contain one or two heteroatoms such as oxygen, nitrogen or sulfur, X and X′ are independently R, halo, —CO₂R, —CN, —NRR′, —NRCOR′, —NO₂, —N₃ or —OR.

17. The method of claim 15 with the structure IVb above wherein: Q and Q′ are independently hydrogen, —CH₂—, —O—, —S—, alkyl, or substituted alkyl, R¹ is hydrogen, alkyl or together with Q may be a cycloalkyl ring, R may be absent, or if present may be—CH₂—, —CO_, —CH₂CH₂—, —CH₂CO—, —CH₂O—, or —CONR—, Y is hydrogen or —OR³, or serves to link the aromatic ring to A as a single bond, ═N— or —NR—, R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring, A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur; R is hydrogen, aryl, arylalkyl, substituted aryl, substituted arylalkyl, alkyl, substituted alkyl, cycloalkyl, substituted cycloalkyl, or heterocycloalkyl, R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur, X and X′ are independently R, halo, —CO₂R, —CN, —NRR′, —NRCOR′, —NO₂, —N₃ or —OR.

18. The method of claim 15 in which Q and Q′ are —CH₂— and R² is —CH₂—.

19. The method of claim 15 in which R¹ is hydrogen.

20. The method of claim 15, wherein Q and Q′ are —CH₂— and R² is —CH₂CH₂—.

21. The method of claim 15, in which Q′ is —CH₂—, R² is —CH₂— and Q is —O— or —S—.

22. The method of claim 15 in which Q is —O—.

23. The method of claim 15 in which Q and Q′ are alkyl and R² is absent.

24. The method of claim 15 in which Q and Q′ are alkyl, R² is absent and R¹ is hydrogen.

25. The method of claim 15 in which Y is —OR³ and A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur.

26. The method of claim 15 in which A is alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur.

27. The method of claim 15 in which A is alkyl, substituted alkyl, cycloalkyl, substituted cycloalkyl, cycloalkylalkyl, a heterocycle or a substituted heterocycle containing one heteroatom such as oxygen, nitrogen or sulfur.

28. The method of claim 15 in which A is —NRR′, R is hydrogen, aryl, arylalkyl, substituted aryl, substituted arylalkyl, alkyl, substituted alkyl, cycloalkyl, substituted cycloalkyl, or heterocycloalkyl, R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and linked to Y by R³ and which may optionally contain one additional heteroatom such as oxygen, nitrogen or sulfur and X and X′ are independently R, halo, —CO₂R, —CN, —NRR′, —NRCOR′, —NO₂, —N₃ or —OR.

29. The method of claim 15 in which A is —NRR′, R is alkyl, substituted alkyl, cycloalkyl, substituted cycloalkyl, or heterocycloalkyl, R′ is hydrogen, alkyl, substituted alkyl, cycloalkyl, substituted cycloalkyl or may join together with R to form a 4- to 8-membered ring, which may be substituted by X and linked to Y by R³ and which may optionally contain one additional heteroatom such as oxygen, nitrogen or sulfur and X and X′ are independently R, halo, —CO₂R, —CN, —NRR′, —NRCOR′, —NO₂, —N₃ or —OR.

30. The method of claim 29 in which A is —NRR′ and R′ is joined together with R to form a 4- to 8-membered ring, which may be substituted by X and linked to Y by R³ and which may optionally contain one additional heteroatom such as oxygen, nitrogen or sulfur and X and X′ are independently R, halo, —CO₂R, —CN, —NRR′, —NRCOR′, —NO₂, —N₃ or —OR.

31. The method of claim 30 in which A is —NRR′, and R′ is joined together with R to form a 5-membered ring, which may be substituted by X and linked to Y by R.sup.3 and which may optionally contain one additional heteroatom such as oxygen, nitrogen or sulfur and X and X′ are independently R, halo, —CO₂R, —CN, —NRR′, —NRCOR′, —NO₂, —N₃ or —OR.

32. The method of claim 31 in which A is —NRR′, and R′ is joined together with R to form a 5-membered ring, which may be substituted by X and linked to Y by R³ and which may optionally contain one additional heteroatom such as oxygen, nitrogen or sulfur and X and X′ are independently R, halo, —CO₂R, —CN, —NRR′, —NRCOR′, —NO₂, —N₃ or —OR.

33. The method of claim 29 in which A is —NRR′, and R′ is joined together with R to form a 5-membered ring, which is linked to Y by R³.

34. The method of claim 30 in which A is —NRR′, and R′ is joined together with R to form a 6-membered ring, which may be substituted by X and linked to Y by R³ and which may optionally contain one additional heteroatom such as oxygen, nitrogen or sulfur and X and X′ are independently R, halo, —CO₂R, —CN, —NRR′, —NRCOR′, —NO₂, —N₃ or —OR.

35. The method of claim 15 in which Y is —OR′.

36. The method of claim 35 in which R³ is hydrogen.

37. The method of claim 15 in which Y is hydrogen.

38. The method of claim 15 in which Y is ═N— or —NR—.

39. The method of claim 15 in which Y is ═N—.

40. The method of claim 37 in which A is —OR, alkyl, substituted alkyl, cycloalkyl, substituted cycloalkyl, cycloalkylalkyl, a heterocycle or a substituted heterocycle containing one or two heteroatoms such as oxygen, nitrogen or sulfur.

41. The method of claim 37 in which A is —NRR′.

42. The method of claim 66 in which A is —OR, alkyl, substituted alkyl, cycloalkyl, substituted cycloalkyl, cycloalkylalkyl, a heterocycle or a substituted heterocycle containing one or two heteroatoms such as oxygen, nitrogen or sulfur.

43. The method of claim 66 in which A is —NRR′.

44. The method of claim 18 in which Y is —OR³ and A is —NRR′.

45. The method of claim 44 in which R¹ is hydrogen.

46. The method of claim 12, wherein said one or more glutamate AMPA receptor modulators excludes one or more ampakines selected from the group consisting of CX516, CX717, S19892, Org24448, Org26576, and GSK729327.

47. The method of claim 12, wherein said one or more glutamate AMPA receptor modulators comprises a compound in FIGS. 1-8.

48. The method of claim 12, wherein said one or more glutamate AMPA receptor modulators comprises a compound in Table 1.

49. The method of claim 12, wherein said one or more glutamate AMPA receptor modulators comprises LiD37 or D1.

50. The method of claim 5, wherein said the method of increasing BDNF expression level or activity in said mammal is not exercise and/or dietary restriction.

51. The method of claim 5, wherein said method comprises administering BDNF or a BDNF analogue to said mammal.

52. The method of claim 51, wherein said method comprises transfecting a neural cell with a construct that expresses a BDNF.

53. The method of claim 5, wherein said maintaining or increasing the BDNF level or activity in said mammal comprises administering to said mammal one or more agents selected from the group consisting of an anti-depressant drug, an anti-anxiolytic drug, an anti-psychotic drug, an acetylcholinesterase inhibitor, a delta- or mu-opioid receptor agonist, epidermal growth factor (EGF), nerve growth factor (NGF).

54. The method of claim 53, wherein said one or more agents comprises a bicyclic or tricyclic antidepressant.

55. The method of claim 53, wherein said one or more agents comprises a selective serotonin reuptake inhibitor (SSRI).

56. The method of claim 53, wherein said one or more agents comprises an antidepressant selected from the group consisting of fluoxetine, desipramine, 2-methyl-6-(phenylethynyl)-pyridine), and Venlafaxine.

57. The method of claim 53, wherein said one or more agents comprises an anxiolytic agent.

58. The method of claim 57, wherein said agent comprises afobazole, Buspirone, lorazepam, diazepam, fluoxetine, eszopiclone, paroxetine, sertaline, citalopram, clomipramine, clonazepram, and St. John's wort.

59. The method of claim 57, wherein said agent comprises an anti-psychotic.

60. The method of claim 59, wherein said agent comprises an agent selected from the group consisting of quetiapine, Chlorpromazine, fluphenazine, perphenazine, prochlorperazine, thioridazine, trifluoperazine, mesoridazine, promazine, triflupromazine, levomepromazine, chlorprothixene, flupenthixol, thiothixene, zuclopenthixol, haloperidol, droperidol, pimozide, melperone, clozapine, olanzapine, risperidone, quetiapine, ziprasidone, amisulpride, paliperidone, cannabidiol, and LY2140023.

61. The method of claim 53, wherein said agent comprises a histone deacetylase inhibitor.

62. The method of claim 61, wherein said agent comprises an agent selected from the group consisting of sodium butyrate, sodium phenylbutyrate, sodium phenylacetate, pivaloyloxymethylbutyrate, pyroxamide, Depsipeptide, Oxamflatin, benzamide derivative MS-275, trichostatin A, suberoylanilide hydroxamic acid, trapoxin A, trapoxin B, Cyl-1, Cyl-2, HC-toxin, WF-3161, chlamydocin, apicidin, MS-275 (previously called MS-27-275), and depudecin.

63. The method of claim 53, wherein said agent comprises an acetylcholinesterase inhibitor.

64. The method of claim 63, wherein agent comprises an agent selected from the group consisting of huperzine A, physostigmine, pyridostigmine, ambenonium, demarcarium, edrophonium, neostigmine, tacrine (tetrahydroaminoacridine), donepezil (a.k.a. E2020), rivastigmine, metrifonate, galantamine, and phenothiazine.

65. The method of claim 53, wherein agent comprises a neuropeptide whose expression is regulated by cocaine or other amphetamine.

66. The method of claim 53, wherein agent comprises cystamine or nictotine.

67. The method of claim 53, wherein agent comprises a monocyclic or bicyclic loop mimetic of BDNF.

68. The method of claim 53, wherein agent comprises estrogen or adrenocorticotropin.

69. The method of claim 53, wherein agent comprises dopamine, norepinephrine, LDOPA, serotonin, or analogues thereof.

70. The method of claim 53, wherein agent comprises Semax.

71. The method of claim 53, wherein agent comprises a compound that increases the activity of BDNF through up-regulating the BDNF receptor.

72. The method of claim 1, wherein said method comprises improving or restoring congnitive function wherein said improved or restored cognitive function is characterized by improved learning ability or memory, reduced autistic-like behavior, improved attention, and/or reduced hypersensitivity to external stimuli.

73. The use of a compound that increases the level or activity of BDNF in a mammal in the manufacture of a medicament for preserving, improving, or restoring cognitive function in mammal having cognitive impairment and/or a learning disability.

74. The use of claim 73, wherein said mammal has a condition selected from the group consisting of Down's syndrome, autism, Rett's syndrome, nonsyndromic X-linked mental retardation, and fragile X syndrome.

75-81. (canceled)

82. The use of claim 73, wherein said compound comprises a high-impact ampakine.

83. The use of claim 73, wherein said compound comprises a high impact ampakine selected from the group consisting of CX516, CX717, and CX691.

84. The use of claim 82, wherein said high impact ampakine is a compound having the structure IVa or IVb, below: 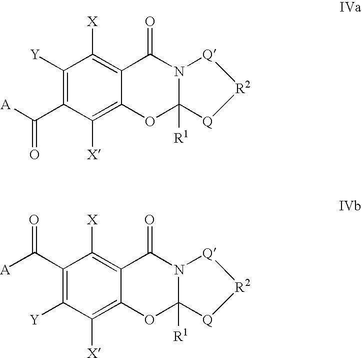
in which:
Q and Q′ are independently hydrogen, —CH₂—, —O—, —S—, alkyl, or substituted alkyl,
R¹ is hydrogen, alkyl or together with Q may be a cycloalkyl ring,
R² may be absent, or if present may be —CH₂—, —CO—, —CH₂CH₂—, —CH₂CO—, —CH₂O—, —CRR′—, or —CONR—,
Y is hydrogen or —OR³, or serves to link the aromatic ring to A as a single bond, ═N— or —NR—,
R³ is hydrogen, alkyl, substituted alkyl, or serves to link the attached oxygen to A by being a lower alkylene such as a methylene or ethylene, or substituted lower alkylene such as —CRR′— linking the aromatic ring to A to form a substituted or unsubstituted 6, 7 or 8-membered ring, or a bond linking the oxygen to A in order to form a 5- or 6-membered ring,
A is —NRR′, —OR, alkyl, substituted alkyl, cycloalkyl, substituted cycloalkyl, cycloalkylalkyl, aryl, substituted aryl, a heterocycle or a substituted heterocycle containing one or two heteroatoms such as oxygen, nitrogen or sulfur,
R is hydrogen, aryl, arylalkyl, substituted aryl, substituted arylalkyl, alkyl, substituted alkyl, cycloalkyl, substituted cycloalkyl, or heterocycloalkyl,
R′ is absent or hydrogen, aryl, arylalkyl, substituted aryl, substituted arylalkyl, alkyl, substituted alkyl, cycloalkyl, substituted cycloalkyl or may join together with R to form a 4- to 8-membered ring, which may be substituted by X and may be linked to Y to form a 6-membered ring and which may optionally contain one or two heteroatoms such as oxygen, nitrogen or sulfur,
X and X′ are independently R, halo, —CO₂R, —CN, —NRR′, —NRCOR′, —NO₂, —N₃ or —OR.

85-89. (canceled)
